(19) United States
(12) Patent Application Publication
(10) Pub. No.: US 2024/0401134 A1
(43) Pub. Date: Dec. 5, 2024

(54) METHODS AND SYSTEMS FOR MEASURING CELL STATES

(71) Applicants: Washington University, St. Louis, MO (US); The Board Of Trustees Of The Leland Stanford Junior University, Stanford, CA (US)

(72) Inventors: Aadel Chaudhuri, St. Louis, MO (US); Irfan Alahi, St. Louis, MO (US); Aaron Newman, Stanford, CA (US)

(73) Assignees: Washington University, St. Louis, MO (US); The Board Of Trustees Of The Leland Stanford Junior University, Stanford, CA (US)

(21) Appl. No.: 17/769,864

(22) PCT Filed: Oct. 18, 2020
(86) PCT No.: PCT/US2020/056218

§ 371 Date: Apr. 18, 2022

Related U.S. Application Data
(60) Provisional application No. 62/916,961, filed on Oct. 18, 2019.

(51) Int. Cl. C12Q 1/6881; C12Q 1/6886; C12Q 1/6888

(52) U.S. Cl. CPC C12Q 1/6881; C12Q 1/6886; C12Q 1/6888; C12Q 2600/154

(57) ABSTRACT

Among the various aspects of the present disclosure is the provision of methods and systems for detecting cell states in a biological sample. An aspect of the present disclosure provides for a method of determining cell type or cell states. In some embodiments, the method comprises providing or having been provided a sample comprising DNA or RNA and generating a methylation profile for the DNA or RNA in the sample or providing or having been provided a methylation profile of the DNA or RNA in the sample.

### CROSS-REFERENCE TO RELATED APPLICATIONS

This application claims priority from U.S. Provisional Application Ser. No. 62/916,961 filed on 18 Oct. 2020, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to methods for detecting cellular states in bodily fluids or nucleic acid mixtures.

### SUMMARY OF THE INVENTION

Among the various aspects of the present disclosure is the provision of methods and systems for detecting cell states.

An aspect of the present disclosure provides for a method of determining cell type or cell states. In some embodiments, the method comprises providing or having been provided a sample comprising DNA or RNA and generating a methylation profile for the DNA or RNA in the sample or providing or having been provided a methylation profile of the DNA or RNA in the sample. In some embodiments, the methylation profile comprises co-associated CpG methylation patterns and methylation haplotype blocks (MHBs) (tightly coupled CpG sites) of the DNA. In some embodiments, the method comprises detecting cell type or cell state comprising counting co-associated CpG methylation patterns in the DNA, wherein co-associated CpG methylation patterns comprises two or more CpGs in the DNA or counting MHBs. In some embodiments, the method comprises assigning the DNA to a cell type or cell state based on reference CpG values or reference MHB values, wherein reference CpG values or reference MHB values are determined from reference cell types or reference cell states. In some embodiments, the method comprises counting DNA molecules assigned to each reference CpG value or reference MHB value, wherein each reference CpG value or reference MHB value corresponds to a cell type or a cell state. In some embodiments, the method further comprises counting known single CpG methylation profiles to increase sensitivity. In some embodiments, the sample is a blood sample. In some embodiments, reference values are differentially methylated CpGs derived from DNA originating from known cell types and known cell states, optionally of bacterial, viral, fungal, or eukaryotic parasitic origin. In some embodiments, the sample is a plasma, tissue, or biopsy sample. In some embodiments, the sample comprises a bodily fluid. In some embodiments, the bodily fluid is selected from whole blood, plasma, urine, saliva, or stool. In some embodiments, the sample does not comprise a solid tissue biopsy. In some embodiments, the DNA or RNA is cell-free DNA or RNA and is plasma-derived. In some embodiments, the method comprises determining cell state-specific signatures by the method of claim 1 or providing or having been provided cell state-specific signatures of the sample. In some embodiments, the DNA or RNA is cell-free and a rare cell type circulating DNA or RNA. In some embodiments, the sample comprises cell-free DNA (cfDNA) or cell-free RNA (cfRNA); and the sample is collected from a tumor microenvironment. In some embodiments, the tumor microenvironment comprises tumor infiltrating leukocytes. In some embodiments, the DNA is cell-free tumor ctDNA. In some embodiments, the subject has been administered immunotherapy prior to providing a sample. In some embodiments, the cell state measured is from DNA from a circulating, cell-free tumor infiltrating leukocyte (TIL) from a tumor microenvironment (TME). In some embodiments, the method comprises profiling TILs according to methylation signatures; and/or determining the proportions of distinct TIL subsets from a cell type-specific methylation profile identified in the cell-free DNA. In some embodiments, DNA is classified as originating from a normal leukocyte cell, a tumor-associated cell, or a tumor infiltrating leukocyte. In some embodiments, the method comprises administering a cancer treatment to the subject (e.g., immunotherapy, chemotherapy, radiation) and measuring cell type and cell state in a sample as an indication of treatment response. In some embodiments, if ctilDNA levels are decreased compared to ctilDNA levels in a responder to immunotherapy, the subject is determined to be at risk for being a non-responder to immunotherapy. In some embodiments, the sample comprises cell-free DNA (cfDNA); and the sample is blood from a subject having, suspected of having, or at risk for having sepsis. In some embodiments, the sample is a blood sample from a subject having, suspected of having, or at risk for having sepsis. In some embodiments, exhausted lymphocyte cell states are measured. In some embodiments, exhausted T cells are measured. In some embodiments, organ-specific cell states or organ-specific cell types are measured. In some embodiments, the DNA originates from an organ, a damaged organ, a T cell, exhausted T cells, an immune cell, a microbe, septic tissue, or a secondary infection site. In some embodiments, if cfDNA analysis detects DNA originating from a microbial pathogen, the subject is diagnosed with an infection or sepsis. In some embodiments, if cfDNA analysis detects reduced cfDNA originating from a microbial pathogen compared to the cfDNA originating from a microbial pathogen, and the subject is administered a treatment (e.g., antibiotic), the subject is determined to be responding to treatment. In some embodiments, if cfDNA analysis detects reduced cfDNA from a microbial pathogen compared to the cfDNA analysis measured at an earlier time, it is determined that the subject is responding to a treatment or an infection is improving. In some embodiments, if cfDNA analysis detects elevated cfDNA from an organ tissue, an infection source is determined to be the organ tissue with elevated detected cfDNA. In some embodiments, if cfDNA analysis detects elevated cfDNA from an organ tissue suspected of being damaged compared to a control, the organ is determined to be damaged. In some embodiments, if cfDNA analysis detects reduced cfDNA from a damaged organ tissue compared to the cfDNA analysis measured at an earlier time, it is determined that the organ damage is improving. In some embodiments, if cfDNA analysis detects elevated cfDNA from an organ tissue suspected of being damaged compared to a control, the organ is determined to be damaged. In some embodiments, if cfDNA analysis detects elevated cfDNA from multiple organ systems compared to a control, the subject is determined to be at risk for multi-organ failure. In some embodiments, if cfDNA analysis detects elevated cfDNA from exhausted T cells or an opportunistic pathogen compared to a control, the subject is determined to be at risk for a secondary infection. In some embodiments, the DNA is cell-free DNA. In some embodiments, instead of DNA, the method uses RNA.

Another aspect of the present disclosure provides for a computer-aided method for detecting at least one abundance of at least one cell identity in a biological sample, the sample comprising DNA. In some embodiments, the method comprises providing a plurality of reads, each read comprising a sequence of the DNA and associated methylation status. In some embodiments, the method comprises providing a CpG library comprising a plurality of entries, each entry comprising a CpG site and a corresponding cell identity, each CpG site comprising a co-associated CpG site, and each corresponding cell identity comprising a cell type or a cell state. In some embodiments, the method comprises transforming, using a computing device, the plurality of reads into a plurality of read assignments according to at least one assignment rule, each read assignment comprising one of a cell identity, a cell-related identity, and an unrelated identity. In some embodiments, the method comprises transforming, using the computing device, the plurality of read assignments into the at least one abundance, each abundance corresponding to one cell identity, each abundance comprising a total number of read assignments comprising the one cell identity. In some embodiments, at least one assignment rule comprises at least one of: transforming, using the computing device, the read into the cell-related identity if the read comprises no more than one CpG site from the plurality of entries of the CpG library; transforming, using the computing device, the read into the cell identity if the read comprises at least two CpG sites from the plurality of entries of the CpG library with the same corresponding cell identity; and/or transforming, using the computing device, the read into the unrelated identity if the read does not comprise any CpG site from the plurality of entries of the CpG library. In some embodiments, the method comprises transforming, using the computing device, each abundance into at least one of a relative abundance and an absolute abundance. In some embodiments, each relative abundance comprises the abundance of one cell identity normalized by the total of all abundances of all cell identities; and/or each absolute abundance comprises the abundance of one cell identity normalized by a sum of the abundance and the total number of read assignments. In some embodiments, providing the plurality of reads further comprises performing bisulfite sequencing or microarray methylation profiling on the DNA. In some embodiments, each CpG site is differentially methylated within cells of one cell identity and each co-associated CpG site comprises a sequence position proximal to at least one additional CpG site with the same corresponding cell identity. In some embodiments, providing the CpG library further comprises providing a plurality of isolated DNA corresponding to one cell identity; performing bisulfite sequencing or microarray methylation profiling on the plurality of isolated cfDNA to obtain a plurality of isolated reads, each isolated read comprising an isolated sequence of an isolated DNA and associated methylation status; performing differential methylated region analysis on the plurality of isolated reads to identify a plurality of candidate CpG sites; and/or assigning a candidate CpG site as an entry of the CpG library for the one cell identity if the candidate CpG site comprises a sequence position proximal to at least one additional candidate CpG site. In some embodiments, the biological sample comprises a bodily fluid. In some embodiments, the bodily fluid is selected from whole blood, plasma, urine, saliva, or stool. In some embodiments, the biological sample does not comprise a solid tissue biopsy. In some embodiments, the DNA is cell-free DNA. In some embodiments, instead of DNA, the method uses RNA.

Yet another aspect of the present disclosure provides for a computing device configured to detect at least one abundance of at least one cell identity in a biological sample, the sample comprising DNA, the computing device comprising at least one processor and a non-volatile computer-readable media, the non-volatile computer-readable media containing instructions executable on the at least one processor to: receive a plurality of reads, each read comprising a sequence of the DNA and associated methylation status; provide a CpG library comprising a plurality of entries, each entry comprising a CpG site and a corresponding cell identity, each CpG site comprising a co-associated CpG site, and each corresponding cell identity comprising a cell type or a cell state; transform the plurality of reads into a plurality of read assignments according to at least one assignment rule, each read assignment comprising one of a cell identity, a cell-related identity, and an unrelated identity; and/or transform the plurality of read assignments into the at least one abundance, each abundance corresponding to one cell identity, each abundance comprising a total number of read assignments comprising the one cell identity. In some embodiments, the at least one assignment rule comprises at least one of transforming, using the computing device, the read into the cell-related identity if the read comprises no more than one CpG site from the plurality of entries of the CpG library; transforming, using the computing device, the read into the cell identity if the read comprises at least two CpG sites from the plurality of entries of the CpG library with the same corresponding cell identity; and/or transforming, using the computing device, the read into the unrelated identity if the read does not comprise any CpG site from the plurality of entries of the CpG library. In some embodiments, the non-volatile computer-readable media further contains instructions executable on the at least one processor to transform each abundance into at least one of a relative abundance and an absolute abundance, wherein: each relative abundance comprises the abundance of one cell identity normalized by the total of all abundances of all cell identities; and/or each absolute abundance comprises the abundance of one cell identity normalized by a sum of the abundance and the total number of read assignments. In some embodiments, each CpG site is differentially methylated within cells of one cell identity and each co-associated CpG site comprises a sequence position proximal to at least one additional CpG site with the same corresponding cell identity. In some embodiments, the biological sample comprises a bodily fluid. In some embodiments, the bodily fluid is selected from whole blood, plasma, urine, saliva, or stool. In some embodiments, the biological sample does not comprise a solid tissue biopsy. In some embodiments, the DNA is cell-free DNA. In some embodiments, instead of DNA, the device detects RNA.

Yet another aspect of the present disclosure provides for a computer-aided method for detecting at least one abundance of at least one cell identity in a biological sample, the sample comprising DNA, the method comprising: providing a plurality of reads, each read comprising a sequence of the DNA and associated methylation status; providing a Methylation Haplotype Block (MHB) library comprising a plurality of entries, each entry comprising an MHB and a corresponding cell identity, each MHB comprising at least two co-associated CpG sites, and each corresponding cell identity comprising a cell type or a cell state; transforming, using a computing device, the plurality of reads into a plurality of read assignments according to at least one assignment rule, each read assignment comprising one of a cell identity, a cell-related identity, and an unrelated identity; and/or transforming, using the computing device, the plurality of read assignments into at least one abundance, each abundance corresponding to one cell identity, each abundance comprising a total number of read assignments comprising the one cell identity. In some embodiments, at least one assignment rule comprises transforming, using the computing device, the read into the cell identity if the read comprises at least one MHB from the plurality of entries of the MHB library with the corresponding cell identity. In some embodiments, the method comprises transforming, using the computing device, each abundance into a relative abundance, wherein each relative abundance comprises the abundance of one cell identity normalized by the total of all abundances of all cell identities. In some embodiments, providing the plurality of reads further comprises performing bisulfite sequencing or microarray methylation profiling on the DNA. In some embodiments, each MHB site comprises at least two differentially methylated CpG sites in proximity to one another within cells of one cell identity. In some embodiments, providing the MHB library further comprises: providing a plurality of isolated DNA corresponding to one cell identity; performing bisulfite sequencing or microarray methylation profiling on the plurality of isolated DNA to obtain a plurality of isolated reads, each isolated read comprising an isolated sequence of the isolated DNA and associated methylation status; performing differential methylated region analysis on the plurality of isolated reads to identify a plurality of candidate CpG sites; and/or assigning each sequence including at least two candidate CpG sites near one another as an MHB corresponding to the one cell identity in the MHB library for the one cell identity. In some embodiments, the biological sample comprises a bodily fluid. In some embodiments, the bodily fluid is selected from whole blood, plasma, urine, saliva, or stool. In some embodiments, the biological sample does not comprise a solid tissue biopsy. In some embodiments, the DNA is cell-free DNA. In some embodiments, instead of DNA, the method uses RNA.

Yet another aspect of the present disclosure provides for a computing device configured to detect at least one abundance of at least one cell identity in a biological sample, the sample comprising DNA, the computing device comprising at least one processor and a non-volatile computer-readable media, the non-volatile computer-readable media containing instructions executable on the at least one processor to: receive a plurality of reads, each read comprising a sequence of the DNA and associated methylation status; receive a Methylation Haplotype Block (MHB) library comprising a plurality of entries, each entry comprising an MHB and a corresponding cell identity, each MHB comprising at least two co-associated CpG sites, and each corresponding cell identity comprising a cell type or a cell state; transform, using a computing device, the plurality of reads into a plurality of read assignments according to at least one assignment rule, each read assignment comprising one of a cell identity, a cell-related identity, and an unrelated identity; and/or transform, using the computing device, the plurality of read assignments into the at least one abundance, each abundance corresponding to one cell identity, each abundance comprising a total number of read assignments comprising the one cell identity. In some embodiments, at least one assignment rule comprises transforming, using the computing device, the read into the cell identity if the read comprises at least one MHB from the plurality of entries of the MHB library with the corresponding cell identity. In some embodiments, the non-volatile computer-readable media further contains instructions executable on the at least one processor to transform each abundance into a relative abundance, wherein each relative abundance comprises the abundance of one cell identity normalized by the total of all abundances of all cell identities. In some embodiments, each MHB site comprises at least two differentially methylated CpG sites in proximity to each other within cells of one cell identity. In some embodiments, the biological sample comprises a bodily fluid. In some embodiments, the bodily fluid is selected from whole blood, plasma, urine, saliva, or stool. In some embodiments, the biological sample does not comprise a solid tissue biopsy. In some embodiments, the DNA is cell-free DNA. In some embodiments, instead of DNA, the device detects RNA.

Yet another aspect of the present disclosure provides for a computer-aided method for detecting at least one abundance of at least two cell identities in a biological sample, the sample comprising DNA, the method comprising: providing a plurality of reads, each read comprising a sequence of the DNA and associated methylation status; providing a signature matrix comprising at least two pluralities of differentially methylated CpG sites, each portion corresponding to each cell identity of the at least two cell identities; and/or deconvolving, using a computing device, the plurality of reads into at least two relative abundances, each relative abundance comprising a portion of one cell identity within the biological sample. In some embodiments, the DNA is cell-free DNA. In some embodiments, instead of DNA, the method uses RNA.

Yet another aspect of the present disclosure provides for a computing device configured to detect at least one abundance of at least two cell identities in a biological sample, the sample comprising DNA, the computing device comprising at least one processor and a non-volatile computer-readable media, the non-volatile computer-readable media containing instructions executable on the at least one processor to receive a plurality of reads, each read comprising a sequence of the DNA and associated methylation status; receive a signature matrix comprising at least two pluralities of differentially methylated CpG sites, each portion corresponding to each cell identity of the at least two cell identities; and deconvolve the plurality of reads into at least two relative abundances, each relative abundance comprising a portion of one cell identity within the biological sample. In some embodiments, the DNA is cell-free DNA. In some embodiments, instead of DNA, the method uses RNA.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. Methylation profiling reveals a consistent TIL-specific signature across colorectal cancer patients, but distinct from peripheral blood leukocytes and tumor epithelial cells from colorectal cancer. Heatmap indicates whole genome bisulfite (WGBS) data of sorted tumor (tum), tumor infiltrating leukocyte (TIL), and peripheral blood leukocyte (PBL) populations from different colorectal cancer patients (columns) followed by differential methylated region (DMR) analysis. The 70 most discriminatory CpG positions (rows) based on methylation status are shown here (blue vs. yellow=hypo-vs. hyper-methylated), indicating stereotypically similar methylation signatures within each of the three populations, but distinct between them.

FIG. 2. LiquidTME detects TIL signal in colorectal cancer blood plasma. Whole genome bisulfite sequencing (WGBS) was applied to plasma cell-free DNA from 13 colorectal cancer (CRC) patients. Sequencing results were deconvolved by CIBERSORTx using the methylation signatures derived from the FIG. 1 analysis. This analytical method is referred to as LiquidTME. (a) Percentages of plasma cell-free DNA comprised of DNA originating from tumor infiltrating leukocytes (TIL) (red), tumor cells (blue), and normal peripheral-blood leukocytes (gray) in patients (Left) and healthy donor samples (Right). (b) CRC vs. healthy donor comparisons of plasma TIL DNA levels (Left) and plasma-derived tumor DNA levels (Right) determined by LiquidTME. Mean is represented by horizontal gray bars; P values are calculated by t test with Welch's correction.

FIG. 3. LiquidTME validation of TIL detection from blood plasma in colorectal cancer. Shown are plasma cfDNA (LiquidTME) results vs. tumor ground-truth for the 9 colorectal cancer (CRC) patients in FIG. 2 with detectable plasma TIL signal. X-axis indicates the fraction of cell-free DNA coming from the specified population (tumor cell vs. TIL vs. PBL), while the Y-axis indicates ground truth proportions from tumor measurement and sequencing (CIBERSORTx deconvolution result multiplied by the sum of longest tumor diameters (SLD)). Data was analyzed in both rank space (shown here with Spearman p) and in non-rank space (Pearson r shown). Significance by both Spearman and Pearson correlation is indicated by P<0.05. There is a strong correlation between the level of tumor signal in plasma compared to ground-truth in tumor (p=0.75, r=0.81). Strikingly, there is also a strong correlation between TIL DNA in plasma and ground-truth in tumor (p=0.71, r=0.70). Indicative of specificity, no positive correlations are apparent when groups are cross-compared with one another.

FIG. 4. LiquidTME measurement of TIL signal from blood plasma correlates strongly with immunotherapy response in melanoma. Plasma cell-free DNA obtained within 4 weeks of immunotherapy start from 12 patients was analyzed by whole genome bisulfite sequencing (WGBS) followed by CIBERSORTx deconvolution using our custom methylation signature matrix (see FIG. 1). Eight of 12 (67%) samples were detectable and are shown here. ctilDNA refers to the percentage of cell-free DNA arising from TILs as calculated by LiquidTME. (a) Melanoma patients are classified as immunotherapy Responders (R) vs. Nonresponders (NR) with ctilDNA percentage indicated in red. (b) Receiver operating characteristic (ROC) analysis of response status based on ctilDNA yields an area under the curve (AUC) of 0.94 with a P value of 0.04, indicating ctilDNA level is serving as a strong classifier of response. (c) Kaplan-Meier analysis of progression-free survival stratified by the optimal cutpoint from the ROC analysis in panel b (12%) stratifies durable responders from rapid early progressors nearly perfectly with hazard ratio of 9.3 and P value of 0.03.

FIG. 5. Differentially methylated CpG sites in purified leukocyte subsets after methylation sequencing. Heatmap indicates whole genome bisulfite (WGBS) data of sorted leukocyte subsets (labeled above) followed by differential methylated region (DMR) analysis. Discriminatory CpG positions (rows) based on methylation status are shown here (blue vs. yellow=hypo-vs. hyper-methylated).

FIG. 6. Ultra-High-Resolution Digital Cytometry via detecting co-associated CpGs within methylation sequencing read-pairs, and using these to assign each read to the matching reference cell type/state. Bulk leukocyte mixtures were sequenced by whole genome bisulfite sequencing (WGBS). Ultra-High-Resolution Digital Cytometry was performed utilizing different numbers of co-associated CpGs per read-pair, and correlated with flow cytometric ground-truth. Pearson r and associated P-value are shown to quantify the strength of the correlation.

FIG. 7. Ultra-High-Resolution Digital Cytometry in Relative and Absolute modes. Bulk leukocyte mixtures were sequenced by whole genome bisulfite sequencing (WGBS). Ultra-High-Resolution Digital Cytometry was performed, with detection of co-associated CpGs per read-pair, followed by assigning each read-pair to its matching reference cell type/state. Results are shown in Relative Mode (left) where the reference-assigned reads are quantified with respect to each other, and Absolute Mode (right) where the reference-assigned fragments were normalized to the total number of unique reads with overlapping CpG positions. In both cases, ultra-high-resolution digital cytometry results were correlated with flow cytometric ground-truth. Pearson r and associated P-value are shown to quantify the strength of the correlation.

FIG. 8 is an illustration showing tumors shed cells and genetic material into the bloodstream (circulation). ctDNA has been previously described, but here it was discovered that that ctilDNA is also present in the peripheral blood.

FIG. 9 is a map showing clonally-related CD8 T cells across tissue compartments and T cell exhausting signatures. RNA-seq reveals TIL-specific cell states, distinct from normal. Left: Single cell RNA sequencing identifies a CD8 TIL gene expression profile, distinct from normal. Clones are distinguished by color. Right: Gene set enrichment analysis showing that exhaustion genes are upregulated in CD8 TILs compared to normal CD8 T cells.

FIG. 10 is a flow chart and a series of graphs showing modeling of ctilDNA detection. Theoretical detection limit modeling of LiquidTME. A) Top: Typical cell-free DNA yield and sequencing depth from 10 mL of blood. Bottom: Following targeted capture and bisulfite sequencing, we conservatively estimate an 80% loss of input molecules, and estimate TIL content given the median level of ctDNA in advanced solid tumor patients (˜1%). Assuming an equal rate of DNA shedding from cancer cells and TILs, and an average of 30% TIL content, we estimate ˜0.4% of cell-free DNA to be ctilDNA. B) Left: Average inferred percentages of major TIL subsets in advanced cancers. Right: Corresponding percentages of TIL subsets in cell-free DNA, based on assumptions in panel a. C) Binomial probability for detecting at least 1 TIL subset based on the number of reporters (DMRs) targeted by the LiquidTME assay given the above assumptions (and 2,000× de-duplicated sequencing depth). D) Same as panel C but showing the expected number of reporters (DMRs) detected as a function of the number targeted. DMR: differentially methylated region. De-duplicated: after removal of duplicate sequencing reads.

FIG. 11. Strategy used to develop and validate the LiquidTME assay and application of LiquidTME clinically.

FIG. 12. Liquid biopsy reveals TME signal in blood plasma. A) TIL and tumor cell signatures were detected in cell-free DNA and tumor, but not peripheral blood mononuclear cells (PBMCs) from 3 CRC patients. B) Inferred TIL and tumor cell levels based on plasma cell-free DNA whole genome bisulfite (WGBS) analysis correlated strongly with flow cytometry and imaging.

FIG. 13. TIL signal measured by LiquidTME correlates with melanoma immunotherapy response. A) ctilDNA levels measured by LiquidTME and stratified by response (DCB=durable clinical benefit, NDB=no durable benefit).

FIG. 14 is an illustration depicting the development of an assay for noninvasive TME profiling and measurement of the technical and in vivo performance.

FIG. 15. Cryopreservation does not introduce epigenetic artifacts. Left: Genomic sites ≥75% methylated in fresh cells vs. cryopreserved frozen cells from the same healthy donor. Jaccard index indicates degree of similarity between the two datasets. Right: Heat map shows methylation rat across the genome in 3 fresh samples, 3 frozen cell samples, and 3 frozen DNA samples from same donor.

FIG. 16. Visualization of differential methylation of the PDCD1 gene in CD8 T cells. Top 3:3 CD8 T TIL samples purified from independent CRC patient tumors. Bottom 7:3 top PBL CD8 T samples are from these same CRC patients; 4 bottom samples are from BLUEPRINT healthy donors.

FIG. 17. Strategy to develop the LiquidTME assay; technical optimization and testing; validation of our technique; and application of LiquidTME clinically.

FIG. 18. Enumeration of leukocyte subsets by CIBERSORT deconvolution of whole blood methylation profiles. (a, b) Scatter plots showing deconvolution performance in relation to flow cytometry in two publicly available datasets: Chakravarthy et al. (a) and Accomando et al. (b).

FIG. 19. LiquidMIDOS will be an all-in-one liquid biopsy technology poised to revolutionize the diagnosis, monitoring, management, and ultimately survival of sepsis patients.

FIG. 20. A deadly hyper-immune response typically dominates during the first few days of sepsis (A). This is followed by a hypo-immune phase that can be self-limited (B) or deadly (C) due to T cell dysfunction/exhaustion which increases the risk of secondary infection, which could potentially be ameliorated with immunotherapy (D). Adapted from Boomer et al, 2014.

FIG. 21. Plasma cfDNA sources in sepsis. Modified from Crowley et al, 2013.

FIG. 22. Liver-derived plasma cell-free DNA levels (Y-axis) in hospitalized patients correlate significantly with serum ALT (X-axis), a gold-standard liver damage biomarker. From Moss et al, 2018.

FIG. 23. Left: Cell-free DNA yield from 10 mL of blood, which following bisulfite conversion and library preparation, undergoes whole genome bisulfite sequencing. Middle: Estimated sources of plasma cell-free DNA and their relative percentages in a sepsis patient, based on Moss et al and Grumaz et al. Right: Binomial probability of detecting each queried cell-free DNA compartment as a function of the number of specific reporters.

FIG. 24. FACS-sorting scheme for exhausted T cells from tissue using canonical surface marker staining.

FIG. 25. Plasma cell-free DNA vs. tumor ground-truth for 9 colorectal cancer patients with cfDNA-detected epithelial signal (Left) and tissue lymphocyte signal (Right). Data analyzed in rank space (shown; Spearman p) and non-rank space (Pearson r).

FIG. 26. Whole genome sequencing detected spiked-in sheared microbial DNA from S. aureus, S. epidermidis, and Adenovirus B (diluted into human plasma between 32 and 1,000 molecules per microliter) with high sensitivity, and specificity as assessed by sequencing 4 independent healthy donor plasma cell-free DNA samples.

FIG. 27 is a block diagram schematically illustrating a system in accordance with one aspect of the disclosure.

FIG. 28 is a block diagram schematically illustrating a computing device in accordance with one aspect of the disclosure.

FIG. 29 is a block diagram schematically illustrating a remote or user computing device in accordance with one aspect of the disclosure.

FIG. 30 is a block diagram schematically illustrating a server system in accordance with one aspect of the disclosure.

### DETAILED DESCRIPTION OF THE INVENTION

The present disclosure is based, at least in part, on the discovery that cell states can be measured in a tissue or bodily fluid. It is noted that the scope of the method is not limited to DNA methylation or plasma-derived cell-free DNA. It can be applied to any sequenced nucleic acid mixture (i.e., DNA or RNA) from any cellular or cell-free DNA source (i.e., any bodily fluid or tissue source). Although examples disclosed here use bisulfite/methylation sequencing, this method can be used with any type of next-generation sequencing or microarray technology known in the art (see e.g., Rajesh et al. 2017-Next-Generation Sequencing Methods; Current Developments in Biotechnology and Bioengineering: Functional Genomics and Metabolic Engineering 2017, Pages 143-158; Moss et al. 2018 Comprehensive human cell-type methylation atlas reveals origins of circulating cell-free DNA in health and disease. Nat Commun 9, 5068; Bumgarner, 2013, Overview of DNA Microarrays: Types, Applications, and Their Future, Volume 101, Issue 1 Pages 22.1.1-22.1.11, for example).

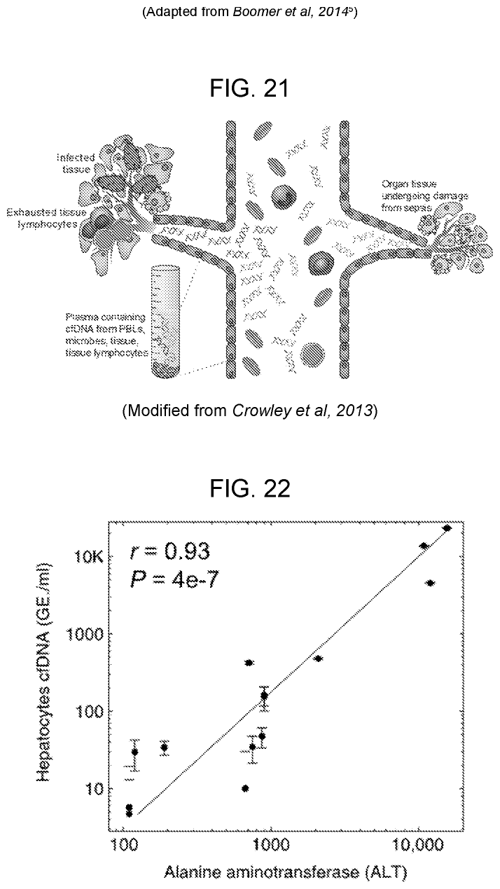

As shown herein, the presently disclosed method enables detection and profiling of a tumor microenvironment (including tumor infiltrating leukocytes and tumor cell states) using a blood based liquid biopsy approach. This is performed through methylation sequencing of plasma-derived cell-free DNA (see e.g., FIG. 8 and FIG. 21 showing genetic material shed from cells, such as cancer cells, microbial cells, infected cells, etc. that can be detected by this method). Individual single cell states are profiled from bulk using either genome-wide or targeted bisulfite sequencing (e.g., leukocyte and tumor cell states by counting or, optionally, deconvolving plasma methylation sequencing data).

This method is not deconvolution, rather it is single molecule counting, which allows us to enumerate and classify molecules (DNA or RNA) into reference bins on a molecule-by-molecule level. As such, the method involves counting, not deconvolution. We start with individual molecules, and by enumerating and classifying them one by one, learn how the full system is comprised molecule-by-molecule. This makes this method extremely high resolution.

In some embodiments, a machine learning model may be used to enumerate and classify DNA or RNA molecules into reference bins. In these embodiments, the machine learning model may be trained using DNA or RNA molecules obtained from isolated cell types or cell states as described herein.

On the other hand, deconvolution starts by looking at the entire bulk sequenced mixture as a whole, then optimally tries to weigh and add cell-type-specific signatures together in order to achieve the mixture-representing matrix. Thus the deconvolution method has intrinsically much lower resolution and is fundamentally different from the disclosed method.

A specific technological advancement implemented is error suppression based on methylation haplotype blocks (“pseudo-UMIs”) (described in Example 1).

This method can enumerate and distinguish cell types and/or cellular states without the need for solid tissue biopsies. “Cellular states” can be defined as context-dependent versions of a given cell type (e.g., normal vs. tumor-associated CD8 T cells). This unique capability allows the presently disclosed noninvasive approach to measure the non-malignant cells within a tumor and distinguish them from their normal tissue counterparts. It is presently believed that this is the first time this has been accomplished. Previous studies have exclusively focused on distinguishing cell types, tissue types, and cancer vs. normal cells—all of these classifications are less granular than cellular states.

The disclosed method is dependent on prior knowledge of cell state-specific signatures (e.g., from known cells). These signatures allow this approach to enumerate specific cell types and cellular states directly from methylation signals in cell-free DNA. Such signatures can be derived by physically isolating cell states of interest by FACS or by inferring them via single-cell bisulfite sequencing. However, these methods have major shortcomings, including the variable loss of specific cell types by tissue dissociation, the sensitivity, and specificity of the antibody panel (needed for FACS), the low amounts of tissue typically obtained from tumor biopsies, etc. We have therefore developed a novel alternative to complement these techniques. Our approach is based on inferring cell state signatures directly from bulk tumor methylation profiles. We can do this via statistical deconvolution in a process that is essentially the inverse of measuring cell composition from bulk methylation profiles (e.g., CIBERSORTx; Newman et al. (2019) Nature Biotechnology (37) 773-782). This novel approach can be used to flexibly generate signatures for nearly any cellular state of interest without antibodies, living cells, or physical cell isolation.

It is noted that the scope of the method is not limited to DNA methylation or plasma-derived cell-free DNA. It can be applied to any sequenced nucleic acid mixture from any cellular or cell-free DNA or RNA source (i.e., any bodily fluid or tissue source).

### Methods and Systems for Noninvasively Measuring Cell States in Bodily Fluids

The present disclosure provides for the noninvasive measurement of measuring cell states in bodily or biological fluids. More specifically, the enumeration of specific cell types and cellular states directly from methylation signals present in cell-free DNA.

As described herein, this technology is capable of identifying a cell type and a cell state in a single cell or a bulk mixture of cells. A cell state can be defined as the phenotype of a cell. The phenotype of a cell can be a ‘homeo-static phenotype’ implying plasticity resulting from a dynamically changing yet characteristic pattern of gene/protein expression.

The methods described herein can be applied to many commercial/biomedical problems, including immunotherapy response assessment, immunotherapy toxicity assessment, response of any tumor to any drug, tracking the tumor microenvironment noninvasively in research, clinical, or commercial applications, and enabling a true liquid biopsy of the tumor that includes both cancer and tumor microenvironment profiling.

This technology can be used in a broad variety of applications using any type of epigenetics data (i.e., whole genome bisulfite sequencing, reduced representation bisulfite sequencing, methylation microarrays, etc.) on any bodily fluid (e.g., urine, saliva, plasma, stool, etc.).

This method enables detection and profiling of the tumor microenvironment (including tumor infiltrating leukocytes and tumor cell states) using a liquid biopsy approach. We do this through methylation sequencing of plasma-derived cell-free DNA, followed by digital cytometry (deconvolution). We profiled individual single cell states from bulk using either genome-wide or targeted bisulfite sequencing (e.g., leukocyte and tumor cell states by deconvolving plasma methylation sequencing data).

Although this method is shown here for detecting cell states and cell types in cell-free DNA, it can also be a useful method for use with nucleic acid sequencing of any length. The nucleic acid can be full length DNA, a DNA fragment, cell-free DNA, RNA, or cell-free nucleic acid fragment assigned to a cell type originating from a tumor cell, an infected cell, a damaged cell, a normal cell, a bacterial cell, an organ or tissue cell, a tissue cell that secretes cfDNA, microbes such as bacteria, viruses (DNA or RNA), fungi, or eukaryotic parasites, for example. In some embodiments, the DNA fragment can be about 300 base pairs or less. It is also noted that the scope of the method is not limited to DNA methylation or plasma-derived cell-free DNA. It can be applied to any sequenced or microarray-profiled nucleic acid mixture from any cellular or cell-free DNA source (i.e., any bodily fluid or tissue source).

As described herein, one or more CpG methylation sites are detected. The CpG methylation sites can be co-associated (e.g., proximal or nearby to each other) between any number of base pairs along the length of a DNA molecule. In some embodiments, the amount of base pairs between co-associated CpGs can be between about 1 base pair (bp) and about 1000 bps (proximal or nearby to each other), between 1 bp and about 500 bps, or between about 1 bp and about 300 bps. For example, the nearby or proximal CpGs can be separated by about 1 bp; about 2 bps; about 3 bps; about 4 bps; about 5 bps; about 6 bps; about 7 bps; about 8 bps; about 9 bps; about 10 bps; about 11 bps; about 12 bps; about 13 bps; about 14 bps; about 15 bps; about 16 bps; about 17 bps; about 18 bps; about 19 bps; about 20 bps; about 21 bps; about 22 bps; about 23 bps; about 24 bps; about 25 bps; about 26 bps; about 27 bps; about 28 bps; about 29 bps; about 30 bps; about 31 bps; about 32 bps; about 33 bps; about 34 bps; about 35 bps; about 36 bps; about 37 bps; about 38 bps; about 39 bps; about 40 bps; about 41 bps; about 42 bps; about 43 bps; about 44 bps; about 45 bps; about 46 bps; about 47 bps; about 48 bps; about 49 bps; about 50 bps; about 51 bps; about 52 bps; about 53 bps; about 54 bps; about 55 bps; about 56 bps; about 57 bps; about 58 bps; about 59 bps; about 60 bps; about 61 bps; about 62 bps; about 63 bps; about 64 bps; about 65 bps; about 66 bps; about 67 bps; about 68 bps; about 69 bps; about 70 bps; about 71 bps; about 72 bps; about 73 bps; about 74 bps; about 75 bps; about 76 bps; about 77 bps; about 78 bps; about 79 bps; about 80 bps; about 81 bps; about 82 bps; about 83 bps; about 84 bps; about 85 bps; about 86 bps; about 87 bps; about 88 bps; about 89 bps; about 90 bps; about 91 bps; about 92 bps; about 93 bps; about 94 bps; about 95 bps; about 96 bps; about 97 bps; about 98 bps; about 99 bps; about 100 bps; about 101 bps; about 102 bps; about 103 bps; about 104 bps; about 105 bps; about 106 bps; about 107 bps; about 108 bps; about 109 bps; about 110 bps; about 111 bps; about 112 bps; about 113 bps; about 114 bps; about 115 bps; about 116 bps; about 117 bps; about 118 bps; about 119 bps; about 120 bps; about 121 bps; about 122 bps; about 123 bps; about 124 bps; about 125 bps; about 126 bps; about 127 bps; about 128 bps; about 129 bps; about 130 bps; about 131 bps; about 132 bps; about 133 bps; about 134 bps; about 135 bps; about 136 bps; about 137 bps; about 138 bps; about 139 bps; about 140 bps; about 141 bps; about 142 bps; about 143 bps; about 144 bps; about 145 bps; about 146 bps; about 147 bps; about 148 bps; about 149 bps; about 150 bps; about 151 bps; about 152 bps; about 153 bps; about 154 bps; about 155 bps; about 156 bps; about 157 bps; about 158 bps; about 159 bps; about 160 bps; about 161 bps; about 162 bps; about 163 bps; about 164 bps; about 165 bps; about 166 bps; about 167 bps; about 168 bps; about 169 bps; about 170 bps; about 171 bps; about 172 bps; about 173 bps; about 174 bps; about 175 bps; about 176 bps; about 177 bps; about 178 bps; about 179 bps; about 180 bps; about 181 bps; about 182 bps; about 183 bps; about 184 bps; about 185 bps; about 186 bps; about 187 bps; about 188 bps; about 189 bps; about 190 bps; about 191 bps; about 192 bps; about 193 bps; about 194 bps; about 195 bps; about 196 bps; about 197 bps; about 198 bps; about 199 bps; about 200 bps; about 201 bps; about 102 bps; about 203 bps; about 204 bps; about 205 bps; about 206 bps; about 207 bps; about 208 bps; about 209 bps; about 210 bps; about 211 bps; about 212 bps; about 213 bps; about 214 bps; about 215 bps; about 216 bps; about 217 bps; about 218 bps; about 219 bps; about 220 bps; about 221 bps; about 222 bps; about 223 bps; about 224 bps; about 225 bps; about 226 bps; about 227 bps; about 228 bps; about 229 bps; about 230 bps; about 231 bps; about 232 bps; about 233 bps; about 234 bps; about 235 bps; about 236 bps; about 237 bps; about 238 bps; about 239 bps; about 240 bps; about 241 bps; about 242 bps; about 243 bps; about 244 bps; about 245 bps; about 246 bps; about 247 bps; about 248 bps; about 249 bps; about 250 bps; about 251 bps; about 252 bps; about 253 bps; about 254 bps; about 255 bps; about 256 bps; about 257 bps; about 258 bps; about 259 bps; about 260 bps; about 261 bps; about 262 bps; about 263 bps; about 264 bps; about 265 bps; about 266 bps; about 267 bps; about 268 bps; about 269 bps; about 270 bps; about 271 bps; about 272 bps; about 273 bps; about 274 bps; about 275 bps; about 276 bps; about 277 bps; about 278 bps; about 279 bps; about 280 bps; about 281 bps; about 282 bps; about 283 bps; about 284 bps; about 285 bps; about 286 bps; about 287 bps; about 288 bps; about 289 bps; about 290 bps; about 291 bps; about 292 bps; about 293 bps; about 294 bps; about 295 bps; about 296 bps; about 297 bps; about 298 bps; about 299 bps; or about 300 bps.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known cellular or tumor composition.

### Computing Systems and Devices

In various aspects, the methods described herein are implemented using computing devices and systems. FIG. 27 depicts a simplified block diagram of a system 800 for implementing the methods described herein. As illustrated in FIG. 27, the system 800 may be configured to implement at least a portion of the tasks associated with the disclosed method. The system 800 may include a computing device 802. In one aspect, the computing device 802 is part of a server system 804, which also includes a database server 806. The computing device 802 is in communication with a database 808 through the database server 806 via a network. The network 850 may be any network that allows local area or wide area communication between the devices. For example, the network 850 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 830 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

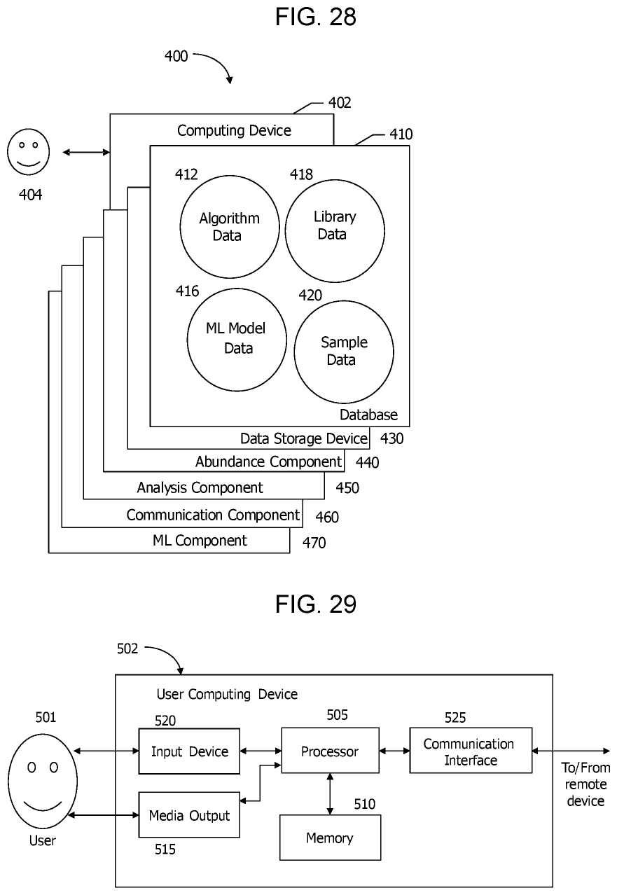

In other aspects, the computing device 802 is configured to perform a plurality of tasks associated with the method of detecting abundances of cell states and/or cell types as described herein. FIG. 28 depicts a component configuration 400 of a computing device 402, which includes a database 410 along with other related computing components. In some aspects, the computing device 402 is similar to computing device 802 (shown in FIG. 27). A user 404 may access components of the computing device 402. In some aspects, the database 420 is similar to the database 808 (shown in FIG. 27).

In one aspect, the database 410 includes library data 418, algorithm data 412, ML model data 416, and sample data 420. In one aspect, the library data 418 includes entries of a library defining characteristics of different cell types or cell states for which the abundance is detected as described herein. Non-limiting examples of library data 418 include entries of a CpG library, entries of a methylation haplotype block (MHB) library, and a signature matrix. As used herein, a CpG library is defined as a plurality of entries in which each entry includes a differentially methylated CpG site indicative of one of the cell types or cell states. In some aspects, the differentially methylated CpG sites are additionally co-associated CpG sites. As used herein, a co-associated CpG site refers to a differentially methylated CpG site characterizing one of the cell types or cell states that is positioned at a distance of no more than about 200 bp from an additional differentially methylated CpG site characterizing the same cell type or cell state. As used herein, an MHB library is defined as a plurality of entries in which each entry includes at least two co-associated CpG sites indicative of one of the cell types or cell states. As used herein, a signature matrix comprises a plurality of differentially methylated CpG sites characterizing all of the at least one cell type or cell state. The signature matrix is used as part of a digital deconvolution method as described herein. Non-limiting examples of suitable digital deconvolution methods include CIBERSORTx.

In various aspects, algorithm data 412 includes any parameters used to implement the methods as described herein. Non-limiting examples of suitable algorithm data 412 include any values of parameters defining the calculation of abundance counts, relative abundances, absolute abundances, and any other relevant parameter. Non-limiting examples of ML model data 416 include any values of parameters defining the machine learning models used to optimize CpG libraries, to perform digital deconvolution, and any other transformation, classification, or other task in accordance with the methods described herein. Non-limiting examples of sample data 420 include any plurality of reads associated with the biological sample analysis in accordance with the methods described herein, including DNA sequences, RNA sequences, DNA methylation sequences, and any other suitable nucleic acid sequence.

The computing device 402 also includes a number of components that perform specific tasks. In the example aspect, the computing device 402 includes a data storage device 430, an abundance component 440, an analysis component 450, an ML component 470, and a communication component 460. The data storage device 430 is configured to store data received or generated by the computing device 402, such as any of the data stored in database 410 or any outputs of processes implemented by any component of the computing device 402. The abundance component 450 is configured to transform the plurality of reads associated with a sample into at least one abundance, at least one relative abundance, at least any absolute abundance, or any combination thereof for each of the at least one cell types or cell states to be detected in accordance with the methods described herein. The analysis component 450 is configured to perform any additional analysis of any of the abundances produced in association with the methods described. Non-limiting examples of additional analyses performed using the analysis component 450 include diagnosis of a disease or disorder such as cancer or sepsis, classification of a patient into a category such as a responder or non-responder to a treatment, determination of a treatment efficacy, and any other suitable analysis. In various aspects, the ML component 470 is configured to implement any of the machine learning model-based transformations and analyses as described herein. Non-limiting examples of transformations or analyses implemented using the ML component 470 include digital deconvolution of the cell types or cell states based on a plurality of reads in a mixed sample. Optimization of a CpG library or an MHB library, or any other suitable transformation or analysis is in accordance with the methods described herein.

The communication component 460 is configured to enable communications of the computing device 402 over a network, such as network 850 (shown in FIG. 27), or a plurality of network connections using predefined network protocols such as TCP/IP (Transmission Control Protocol/Internet Protocol).

FIG. 29 depicts a configuration of a remote or user computing device 502, such as the user computing device 830 (shown in FIG. 27). The computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser, and client application. Web browsers enable users 501 to display and interact with media and other information typically embedded on a web page or a website from a web server. A client application allows users 501 to interact with a server application associated with, for example, a vendor or business.

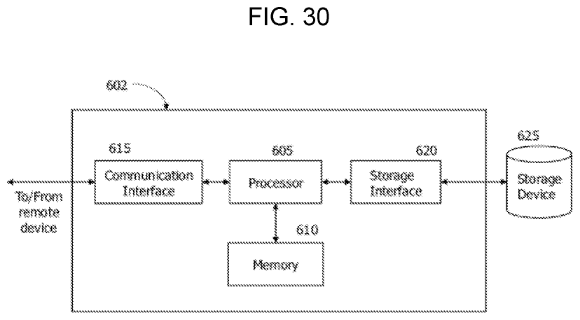

FIG. 30 illustrates an example configuration of a server system 602. Server system 602 may include, but is not limited to, database server 806 and computing device 802 (both shown in FIG. 27). In some aspects, server system 602 is similar to server system 804 (shown in FIG. 27). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 830 (shown in FIG. 27) or another server system 602. For example, communication interface 615 may receive requests from user computing device 830 via a network 850 (shown in FIG. 27).

Processor 605 may also be operatively coupled to a storage device 625. Storage device 625 may be any computer-operated hardware suitable for storing and/or retrieving data. In some aspects, storage device 625 may be integrated in server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 29) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local, remote, o cloud-based processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to: images or frames of a video, object characteristics, and object categorizations. Data inputs may further include: sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a type of motion, a diagnosis based on motion of an object, motion analysis of an object, and trained model parameters ML outputs may further include: speech recognition, image or video recognition, functional connectivity data, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a part of a transaction. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above. For example, a ML module may receive training data comprising customer identification and geographic information and an associated customer category, generate a model that maps customer categories to customer identification and geographic information, and generate a ML output comprising a customer category for subsequently received data inputs including customer identification and geographic information.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In one aspect, a ML module receives unlabeled data comprising customer purchase information, customer mobile device information, and customer geolocation information, and the ML module employs an unsupervised learning method such as “clustering” to identify patterns and organize the unlabeled data into meaningful groups. The newly organized data may be used, for example, to extract further information about a customer's spending habits.

In yet another aspect, ML methods and algorithms are directed toward reinforcement learning, which involves optimizing outputs based on feedback from a reward signal. Specifically, ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, a ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware, or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for storage of a computer program.

In one aspect, a computer program is provided, and the program is embodied on a computer readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: LiquidTME: Liquid Biopsy for Immune Checkpoint Inhibitor (ICI) Response Prediction

This example describes a liquid biopsy of the tumor microenvironment for early immunotherapy response assessment. Immunotherapy transformed modern cancer treatment and improved cancer survival. Immunotherapy “takes the breaks” off tumor immune cells (TILs) to improve cancer cell killing. TILs in the tumor microenvironment (TME) play a critical role in response to therapy. Many patients do not respond to immunotherapy. There are five classes of leukocytes (white blood cells) that coordinate to provide defense against infectious disease (e.g., neutrophils, eosinophils, basophil, monocyte, or lymphocyte). Some subsets can include naïve and memory CD8 T cells and CD4 T cells, NK cells, naïve and memory B cells, monocytes/macrophages, and granulocytes.

The following example and the present disclosure provides for a solution to the problem of assessing response to treatment early. Early imaging assessment is challenging and confounded by factors like pseudoprogression. Other leading prediction measures like tumor PDL1, TMB, and tumor gene expression profiling are not sensitive or specific enough. Currently, there is no reliable way to predict immunotherapy response early.

Here is disclosed a solution to this problem: liquid biopsy of the tumor microenvironment (LiquidTME). The solution is to measure levels/activity of tumor immune cells themselves. Conventional repeated invasive biopsies are impractical and biopsies are subject to sampling bias. Here is described a liquid biopsy approach to do this, termed LiquidTME.

### Co-Associated CpG Methylation Patterns

CpGs adjacent to each other have been shown to share similar methylation patterns due to locally coordinated activity of methylation enzymes and CpGs function at a block level within promoters to regulate gene transcription. We utilized this concept in our ultra-sensitive method for internal error correction, corroborating the methylation status of a CpG site in a single sequenced DNA molecule by examining its adjacent CpGs as well.

### Counting Co-Associated CpG Methylation Patterns at the Single Molecule Level

1. Identify differentially methylated CpGs in purified reference cell types/states after methylation sequencing.
2. Assign sequencing reads from a bulk mixture to each reference cell type/state by tracking co-associated CpGs (based on detection of cell type/state-specific co-associated CpGs at the individual read/molecule level).
3. Count the number of reads per cell type/state to determine their abundances in the bulk mixture. This is ultra-high-resolution digital cytometry at the single molecule level, which enables LiquidTME's high performance.

### BACKGROUND

Cancer is the second most common cause of death in the United States¹ and immunotherapy is a powerful way to treat advanced stages of disease^{2,3}. However, only a fraction of patients respond initially⁴, and in many cases an initial response is not durable⁵. CT imaging is the standard-of-care method for assessing immunotherapy response^{6,7}, however early imaging assessment is unreliable^{8,9}. We currently have no reliable way to predict immunotherapy response early.

Tumors shed cells and genetic material into the circulation (see e.g., FIG. 8). Previously, liquid Biopsies have been applied to ctDNA, CTCs, but not tumor infiltrating leukocytes (TILs). “ctilDNA” are cell-free DNA arising from TILs. This platform, LiquidTME, profiles and measures ctilDNA. The outcome is early immunotherapy response prediction.

Tumor infiltrating leukocytes (TILs) in the tumor microenvironment (TME) determine a patient's response to immunotherapy¹⁰⁻²², enabling tumor cell killing when potentiated^{3,23}. Several groups have shown that early assessment of TILs by invasive biopsy in melanoma patients on immune checkpoint blockade is informative of therapeutic response^{16,20-22,24}. Although TILs can be assessed by invasive biopsy, it is challenging and potentially dangerous to monitor TILs during treatment via repeated invasive biopsies^{25,26}. Moreover, unlike noninvasive liquid biopsies, invasive solid tumor biopsies are subject to sampling bias which can confound results²⁷⁻³⁰. There are no methods available to measure global TIL content in a non-invasive liquid biopsy manner.

We hypothesized that liquid biopsy analysis of methylation signatures in plasma cell-free DNA will enable accurate quantitation of TILs and reliably predict immunotherapy response. Supporting that TILs have a distinct epigenomic profile from normal leukocytes, Philip et al. showed that tumor infiltrating CD8 T cells have a distinct chromatin profile compared to normal CD8 T cells³¹. TILs, both myeloid and lymphoid, have also been shown to have distinct gene expression profiles from normal leukocytes by single cell RNA sequencing³²⁻³⁴. Our novel data also show that TILs have a distinct methylation profile compared to normal leukocytes and tumor cells, allowing us to quantify them via cell-free DNA liquid biopsy.

In addition to data support, we have expertise in cell-free DNA analysis, having published the ability to detect ultra-low levels of circulating tumor DNA, low enough to detect solid tumor molecular residual disease and infer tumor mutational burden³⁵⁻³⁷. We also developed the deconvolution technology CIBERSORTx, which can infer relative abundances of individual cell states from bulk sequencing data³⁸ and is based on the most widely validated deconvolution model in the field³⁹. Our experience with ultra-sensitive cell-free DNA analysis, state-of-the-art sequencing deconvolution, and translational research applying these technologies will facilitate the development of a novel liquid biopsy method called LiquidTME to analyze TILs noninvasively and improve immunotherapy response prediction.

We developed LiquidTME for any cancer or disease state and showcase it here for colorectal cancer and melanoma pre-treatment to detect cell states noninvasively and predict response to different types of treatment including immune checkpoint blockade. We hypothesized that LiquidTME will enable sensitive TIL quantitation and predict therapeutic response better than leading technologies. Furthermore, LiquidTME will complement current efforts being undertaken toward early cancer detection using cell-free DNA⁴⁰. Our work will allow researchers for the first time to specifically assess TILs without requiring invasive tumor biopsy. Moreover, the principles established here should generalize to nearly any disease etiology and therapy type, opening the door to routine, noninvasive TIL assessment in research and clinical settings.

### Data

Methylation Profiles Accurately Distinguish TILs from PBLs and Tumor Cells

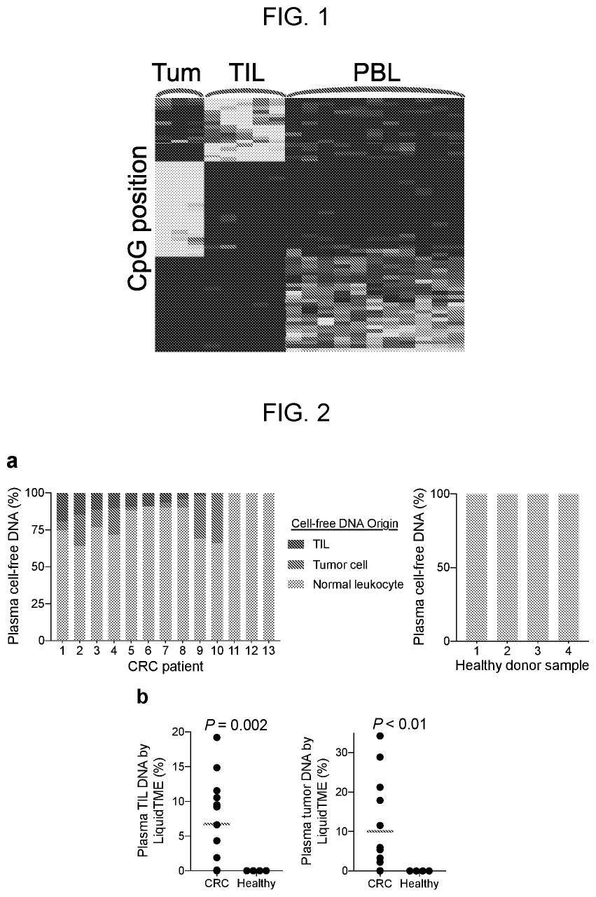

We began by asking if stereotypic epigenomic differences were apparent between tumor infiltrating leukocytes (TILs) and normal peripheral blood leukocytes (PBLs), as suggested by recent scRNA-seq and ATAC-seq data³²⁻³⁴. We thus performed flow cytometry and isolated Epcam+ tumor cells, CD45+ TILs, and CD45+ PBLs from 10 patients with metastatic colorectal cancer (CRC). We performed whole genome bisulfite sequencing (WGBS) on each sample, followed by differential methylated region (DMR) analysis, and identified the 70 most differentially methylated CpG positions (FIG. 1). This revealed that TILs have a distinct methylation profile compared to normal PBLs and tumor cells, suggesting we can use methylation sequencing to quantify TILs.

As such, it was shown that TILs have a distinct methylation profile by methylation profiling of sorted cells (see e.g., FIG. 1) (whole genome bisulfite sequencing (WGBS) on sorted colorectal cancer samples) and differential methylated region (DMR) analysis reveals a TIL-specific methylation pattern.

TIL Signatures are Detected in Plasma Cell-Free DNA from CRC Patients

It was next queried whether TIL signal can be detected in cell-free DNA using a liquid biopsy technology that we call LiquidTME. To do this, we isolated plasma cell-free DNA (cfDNA) from 13 patients with metastatic CRC and performed WGBS on an Illumina NovaSeq S4 flow cell targeting 65 genome-wide coverage. We deconvolved this data by querying the specific TIL vs. PBL vs. tumor cell signatures shown in FIG. 1 using CIBERSORTx. Using this approach (which we call LiquidTME) even at this low sequencing depth, we were able to detect TIL signal from blood plasma in 9 of 13 patients (FIG. 2A). Indicative of specificity, 4 healthy donor plasma samples processed and analyzed in the same way showed no evidence of TIL or tumor DNA signal. Thus, plasma TIL and tumor DNA levels using our LiquidTME approach were significantly higher in CRC patients than in healthy donor controls (FIG. 2B). Our data demonstrate superb methodological sensitivity and specificity. As such, it was shown that LiquidTME in CRC detects ctilDNA in CRC plasma (see e.g., FIG. 2A), evidenced by ctilDNA and tumor signal detected in plasma cell-free DNA from colorectal cancer patients, no ctilDNA or tumor signal detected in plasma cell-free DNA from 12 healthy donor samples, and increased ctilDNA levels in patients compared to healthy controls (see e.g., FIG. 2B).

TIL Levels Detected by LiquidTME in Plasma Cell-Free DNA Correlate with Tumor Ground-Truth

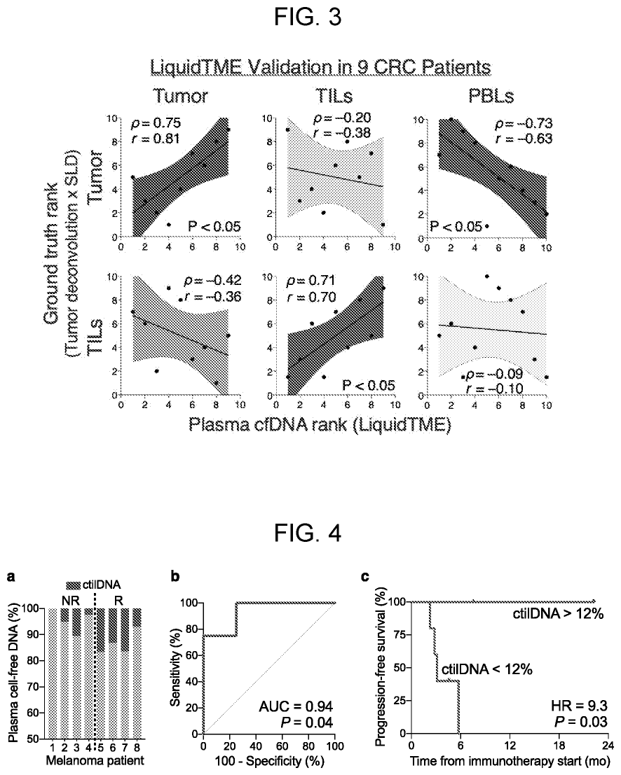

We next queried whether the level of TIL signal detected by LiquidTME correlates with tumor ground-truth. To answer this, we correlated LiquidTME results for the 9 detectable CRC patients discussed above with tumor ground-truth. Strikingly, TIL DNA levels in plasma cfDNA correlated strongly and significantly with tumor ground-truth (Spearman p=0.71, Pearson r=0.70, P<0.05) (FIG. 3). Indicative of specificity, LiquidTME-derived TIL levels did not correlate with ground-truth PBL or tumor cell fractions. As such, it was shown that ctilDNA in plasma correlates with tumor ground truth (see e.g., FIG. 3).

TIL signatures in plasma predict immunotherapy response in melanoma We next applied our LiquidTME assay in a pilot setting to melanoma patients treated with immune checkpoint blockade. To do this, we analyzed banked pre- and early on-treatment plasma samples from 12 patients with metastatic melanoma with on-treatment samples acquired within a month of starting immune checkpoint blockade. The response rate for this pilot cohort was 58%. Applying LiquidTME as described above to cfDNA extracted from each of these samples, we achieved ˜70% assay sensitivity. Interestingly, quantifying plasma TIL DNA as a percentage of total cfDNA revealed that responders had a higher plasma TIL DNA level than nonresponders (FIG. 4A). Indeed, ROC analysis demonstrated a striking result with area under the curve (AUC) of 0.94 (FIG. 4B), with the optimal plasma TIL DNA cutpoint being 12%. Applying this cutpoint to 8 assay-detected patients stratified long-term survivors from short-term progressors nearly perfectly by Kaplan-Meier progression-free survival analysis (HR=9.3, P=0.03) (FIG. 4C). Our data demonstrate that quantifying plasma TIL DNA in melanoma patients can accurately predict immunotherapy response.

As such it has been shown that LiquidTME can also be applied to melanoma immunotherapy response (see e.g., FIG. 4) as shown by the application of LiquidTME to Melanoma Plasma Samples Collected Pre- or Early On-Immunotherapy. It was shown that ctilDNA or tumor signal detected in 8 of 13 samples (62%) and ctilDNA levels in cell-free DNA correlate strongly with durable response among these 8 detectable patients.

### Ultra-High-Resolution Digital Cytometry

We developed a completely novel technology for ultra-high-resolution digital cytometry in order to achieve the sensitivity necessary for LiquidTME to perform robustly. Specifically, we track differentially methylated CpGs at the single molecule level, while utilizing the methylation status of adjacent CpGs (“co-associated CpGs”) for internal error correction.

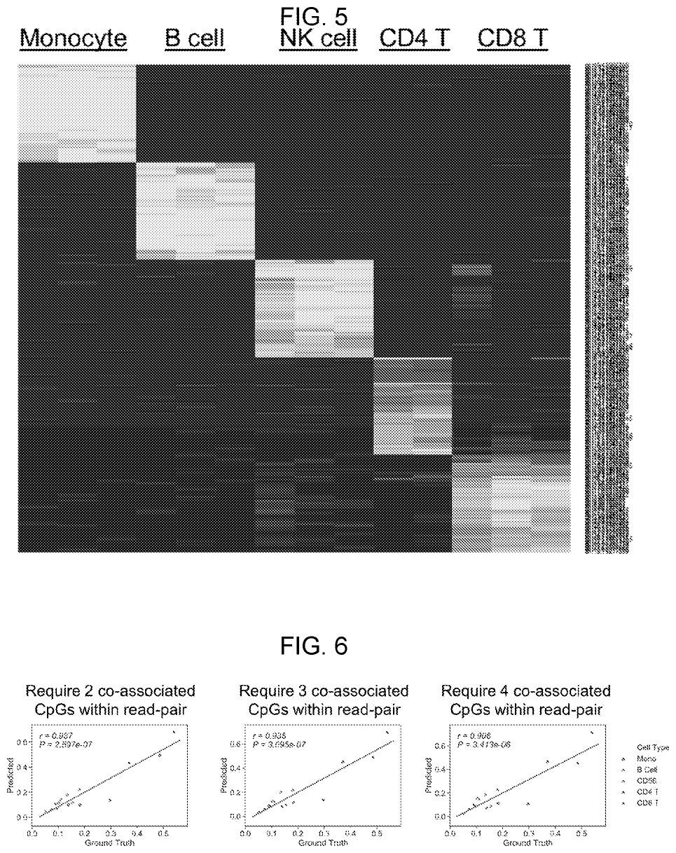

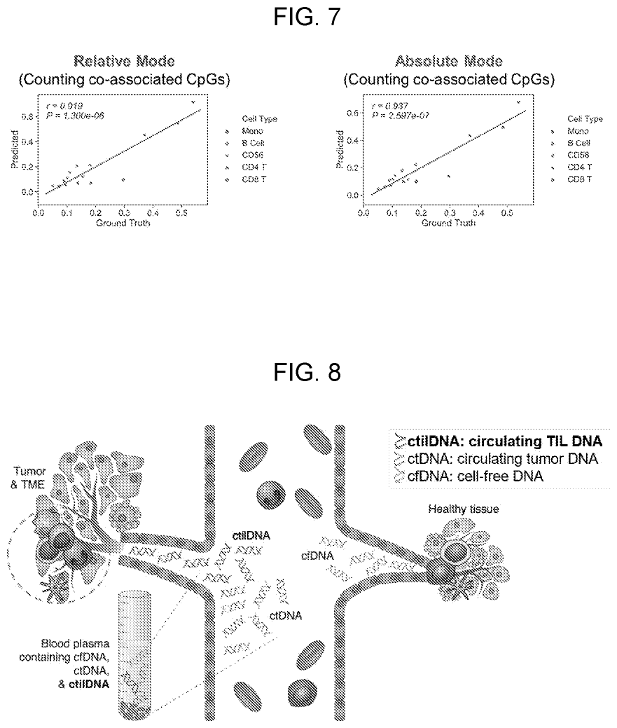

The steps of our technology are as follows:
1. Identify differentially methylated CpGs in methylation sequencing or microarray data of purified reference cell types/states (FIG. 5). FIG. 5 depicts whole genome bisulfite sequencing (WGBS) methylation data showing differentially methylated CpGs in sorted leukocyte subsets.
2. Methylation profiling (i.e., WGBS) of bulk cellular mixture. Assign individual sequencing reads (or read-pairs if performing paired-end sequencing) from this bulk mixture to each of the reference cell types/states from step 1 by identifying differentially methylated CpGs at the single molecule level (examined on a per-read or per-read-pair basis), after confirming that adjacent CpGs within the same read/read-pair have the same methylation status (“co-associated CpGs”). We investigated this with different number of required co-associated CpGs (i.e., 2, 3, 4 per read-pair) and showed similar high performance compared to ground-truth flow cytometry regardless of the parameter value (FIG. 6). Our method correlates well with flow cytometric ground truth across a range of co-associated CpGs (FIG. 6).
3. After assigning individual DNA molecules (sequencing reads/read-pairs) in the bulk mixture as per step 2, we can quantify how the bulk mixture is comprised of the individual reference cell types/states (ultra-high-resolution digital cytometry). We do this either by counting the binned/assigned reads with respect to one another (relative mode) or by normalizing the number of reference-assigned fragments to the total number of unique reads with overlapping CpG positions (absolute mode). When we applied this method to bulk leukocyte mixtures, we capably quantified the individual leukocyte subsets making up these mixtures, which correlated strongly with flow cytometric ground-truth (FIG. 7). Our method correlates well with flow cytometric ground truth in both Relative and Absolute read-counting mode (FIG. 7). Our single molecule read-counting method is an ultra-high-resolution digital cytometry method for tracking cell types/states.

Overall our ultra-high-resolution digital cytometry technology for quantifying and tracking cell types/states exhibits high performance, is ultra-sensitive, and can be applied to cell-free DNA, enabling noninvasive detection of rare cell states, such as those arising from the tumor microenvironment, important for predicting immunotherapy response via our LiquidTME method.

### Innovation: High-Resolution Digital Cytometry at the Single Molecule Level

1. Identify differentially methylated co-associated CpGs by DMR analysis of purified reference cell types/states.
2. Assign sequencing reads from bulk mixture to each cell type/state (based on detection of cell type/state-specific co-associated CpGs at the individual read level).
3. Count the number of reads per cell type/state to determine their relative abundances in the bulk mixture.

### Innovation: Alternative Approach Using Methylation Haplotype Blocks

1. Linkage disequilibrium principles to identify tightly coupled CpG sites (“Methylation Haplotype Blocks”).
2. Divide epigenome into ˜150,000 methylation haplotype blocks (MHBs) of tightly coupled CpG sites.
3. Reference profile of each sequenced purified cell type/state by looking at differentially methylated MHBs.
4. Assign sequencing reads from bulk mixture to each cell type/state-specific MHB (based on MHBs identified at the individual read level).
5. Count the number of reads per cell type/state to determine their relative abundances in the bulk mixture.

### Significance

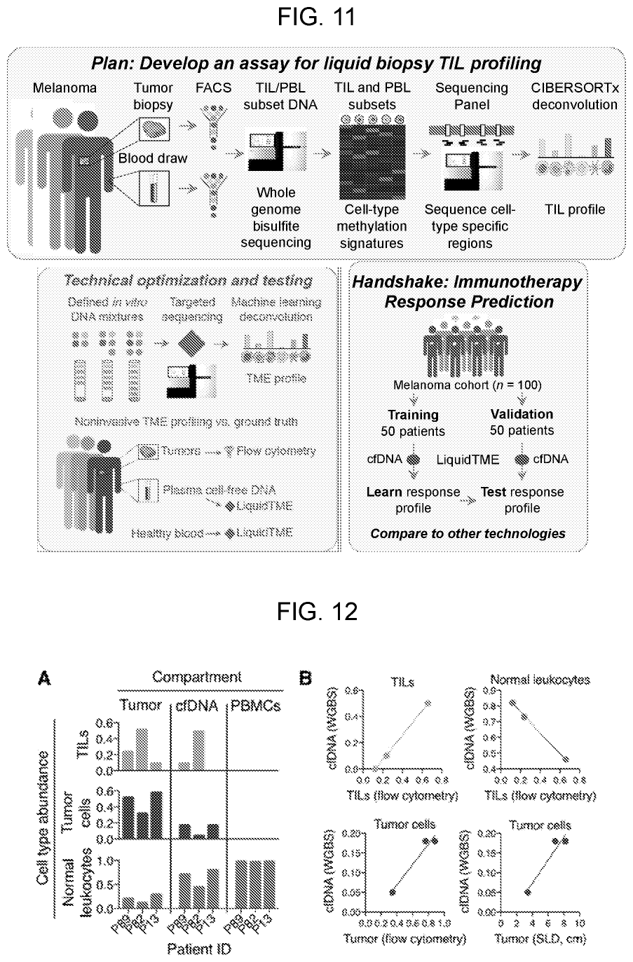

This is the first method to profile TILs through liquid biopsy (see e.g., FIG. 11). LiquidTME will enable early immunotherapy response prediction, make serial profiling of TILs practical, and improve clinical decision-making and patient survival.

### SUMMARY

The described technology enables robust ultra-high-resolution digital cytometry to measure cell states from methylation sequencing data. Given its ultra-sensitivity, it can be applied to cell-free DNA, enabling noninvasive detection of rare cell states, such as those in the tumor microenvironment. The approach, called LiquidTME serves as a robust early predictor of immunotherapy response in cancer patients through ultra-sensitive tumor infiltrating leukocyte detection.

### REFERENCES

1. Siegel, R. L., Miller, K. D. & Jemal, A. Cancer statistics, 2019. CA Cancer J Clin 69, 7-34 (2019).
2. Postow, M. A., Callahan, M. K. & Wolchok, J. D. Immune Checkpoint Blockade in Cancer Therapy. J Clin Oncol 33, 1974-1982 (2015).

3 Ribas, A. & Wolchok, J. D. Cancer immunotherapy using checkpoint blockade. Science 359, 1350-1355 (2018).
4. Yarchoan, M., Hopkins, A. & Jaffee, E. M. Tumor Mutational Burden and Response Rate to PD-1 Inhibition. N Engl J Med 377, 2500-2501 (2017).
5. Zappasodi, R., Wolchok, J. D. & Merghoub, T. Strategies for Predicting Response to Checkpoint Inhibitors. Curr Hematol Malig Rep 13, 383-395 (2018).
6. Hodi, F. S., et al. Evaluation of Immune-Related Response Criteria and RECIST v1.1 in Patients With Advanced Melanoma Treated With Pembrolizumab. J Clin Oncol 34, 1510-1517 (2016).
7. Wolchok, J. D., et al. Guidelines for the evaluation of immune therapy activity in solid tumors: immune-related response criteria. Clin Cancer Res 15, 7412-7420 (2009).
8. Chiou, V. L. & Burotto, M. Pseudoprogression and Immune-Related Response in Solid Tumors. J Clin Oncol 33, 3541-3543 (2015).
9. Nishino, M. Immune-related response evaluations during immune-checkpoint inhibitor therapy: establishing a “common language” for the new arena of cancer treatment. J Immunother Cancer 4, 30 (2016).
10. Fridman, W. H., Pages, F., Sautes-Fridman, C. & Galon, J. The immune contexture in human tumours: impact on clinical outcome. Nat Rev Cancer 12, 298-306 (2012).
11. Gajewski, T. F., Schreiber, H. & Fu, Y. X. Innate and adaptive immune cells in the tumor microenvironment. Nat Immunol 14, 1014-1022 (2013).
12. Pang, Y. L., et al. The immunosuppressive tumor microenvironment in hepatocellular carcinoma. Cancer Immunol Immunother 58, 877-886 (2009).
13. Spranger, S. & Gajewski, T. F. Impact of oncogenic pathways on evasion of antitumour immune responses. Nat Rev Cancer 18, 139-147 (2018).
14. Thommen, D. S. & Schumacher, T. N. T Cell Dysfunction in Cancer. Cancer Cell 33, 547-562 (2018).
15. Jimenez-Sanchez, A., et al. Heterogeneous Tumor-Immune Microenvironments among Differentially Growing Metastases in an Ovarian Cancer Patient. Cell 170, 927-938 e920 (2017).
16. Mlecnik, B., et al. The tumor microenvironment and Immunoscore are critical determinants of dissemination to distant metastasis. Sci Transl Med 8, 327ra326 (2016).
17. Rizvi, H., et al. Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol 36, 633-641 (2018).
18. Rooney, M. S., Shukla, S. A., Wu, C. J., Getz, G. & Hacohen, N. Molecular and genetic properties of tumors associated with local immune cytolytic activity. Cell 160, 48-61 (2015).
19. Wang, C., Singer, M. & Anderson, A. C. Molecular Dissection of CD8 (+) T-Cell Dysfunction. Trends Immunol 38, 567-576 (2017).
20. Chen, P. L., et al. Analysis of Immune Signatures in Longitudinal Tumor Samples Yields Insight into Biomarkers of Response and Mechanisms of Resistance to Immune Checkpoint Blockade. Cancer Discov 6, 827-837 (2016).
21. Cooper, Z. A., et al. Distinct clinical patterns and immune infiltrates are observed at time of progression on targeted therapy versus immune checkpoint blockade for melanoma. Oncoimmunology 5, e1136044 (2016).
22. Roh, W., et al. Integrated molecular analysis of tumor biopsies on sequential CTLA-4 and PD-1 blockade reveals markers of response and resistance. Sci Transl Med 9 (2017).
23. Tumeh, P. C., et al. PD-1 blockade induces responses by inhibiting adaptive immune resistance. Nature 515, 568-571 (2014).
24. Riaz, N., et al. Tumor and Microenvironment Evolution during Immunotherapy with Nivolumab. Cell 171, 934-949 e915 (2017).
25. Shyamala, K., Girish, H. C. & Murgod, S. Risk of tumor cell seeding through biopsy and aspiration cytology. J Int Soc Prev Community Dent 4, 5-11 (2014).
26. Yam, C., et al. Risk of needle-track seeding with serial ultrasound guided biopsies in triple negative breast cancer. Cancer Res 78 (2018).
27. Abbosh, C., et al. Phylogenetic ctDNA analysis depicts early-stage lung cancer evolution. Nature 545, 446-451 (2017).
28. Biswas, D., et al. A clonal expression biomarker associates with lung cancer mortality. Nat Med 25, 1540-1548 (2019).
29. Jamal-Hanjani, M., et al. Tracking the Evolution of Non-Small-Cell Lung Cancer. N Engl J Med 376, 2109-2121 (2017).
30. Joshi, K., et al. Spatial heterogeneity of the T cell receptor repertoire reflects the mutational landscape in lung cancer. Nat Med 25, 1549-1559 (2019).
31. Philip, M., et al. Chromatin states define tumour-specific T cell dysfunction and reprogramming. Nature 545, 452-456 (2017).
32. Azizi, E., et al. Single-Cell Map of Diverse Immune Phenotypes in the Breast Tumor Microenvironment. Cell 174, 1293-1308 e1236 (2018).
33. Zheng, C., et al. Landscape of Infiltrating T Cells in Liver Cancer Revealed by Single-Cell Sequencing. Cell 169, 1342-1356 e1316 (2017).
34. Zilionis, R., et al. Single-Cell Transcriptomics of Human and Mouse Lung Cancers Reveals Conserved Myeloid Populations across Individuals and Species. Immunity 50, 1317-1334 e1310 (2019).
35. Azad, T. D., et al. Circulating Tumor DNA Analysis for Detection of Minimal Residual Disease After Chemoradiotherapy for Localized Esophageal Cancer. Gastroenterology (2019).
36. Chaudhuri, A. A., et al. Early Detection of Molecular Residual Disease in Localized Lung Cancer by Circulating Tumor DNA Profiling. Cancer Discov 7, 1394-1403 (2017).
37. Chin, R. I., et al. Detection of Solid Tumor Molecular Residual Disease (MRD) Using Circulating Tumor DNA (ctDNA). Mol Diagn Ther 23, 311-331 (2019).
38. Newman, A. M., et al. Determining cell type abundance and expression from bulk tissues with digital cytometry. Nat Biotechnol (2019).
39. Newman, A. M., et al. Robust enumeration of cell subsets from tissue expression profiles. Nat Methods 12, 453-457 (2015).
40. Shen, S. Y., et al. Sensitive tumour detection and classification using plasma cell-free DNA methylomes. Nature 563, 579-583 (2018).
41. Juhling, F., et al. metilene: fast and sensitive calling of differentially methylated regions from bisulfite sequencing data. Genome Res 26, 256-262 (2016).
42. Xu, R. H., et al. Circulating tumour DNA methylation markers for diagnosis and prognosis of hepatocellular carcinoma. Nat Mater 16, 1155-1161 (2017).
43. Newman, A. M., et al. Integrated digital error suppression for improved detection of circulating tumor DNA. Nat Biotechnol 34, 547-555 (2016).
44. Newman, A. M., et al. An ultrasensitive method for quantitating circulating tumor DNA with broad patient coverage. Nat Med 20, 548-554 (2014).
45. Chabon, J. J., et al. Integrating genomic features for non-invasive early lung cancer detection. Nature 580, 245-251 (2020).
46. Phallen, J., et al. Direct detection of early-stage cancers using circulating tumor DNA. Sci Transl Med 9 (2017).
47. Thorsson, V., et al. The Immune Landscape of Cancer. Immunity 48, 812-830 e814 (2018).
48. Zill, O. A., et al. The Landscape of Actionable Genomic Alterations in Cell-Free Circulating Tumor DNA from 21,807 Advanced Cancer Patients. Clin Cancer Res 24, 3528-3538 (2018).
49. Cohen, J. Statistical power analysis for the behavioral sciences, (L. Erlbaum Associates, Hillsdale, N. J., 1988).
50. Borcoman, E., et al. Novel patterns of response under immunotherapy. Ann Oncol 30, 385-396 (2019).
51. Pons-Tostivint, E., et al. Comparative Analysis of Durable Responses on Immune Checkpoint Inhibitors Versus Other Systemic Therapies: A Pooled Analysis of Phase III Trials. Jco Precis Oncol 3 (2019).
52. Roach, C., et al. Development of a Companion Diagnostic PD-L1 Immunohistochemistry Assay for Pembrolizumab Therapy in Non-Small-cell Lung Cancer. Appl Immunohistochem Mol Morphol 24, 392-397 (2016).
53. Zaretsky, J. M., Blum, S. M., Faja, L. R., Emerson, R. O. & Ribas, A. TCR use and cytokine response in PD-1 blockade. Journal of Clinical Oncology 33 (2015).
54. Goodman, A. M., et al. Tumor Mutational Burden as an Independent Predictor of Response to Immunotherapy in Diverse Cancers. Mol Cancer Ther 16, 2598-2608 (2017).
55. Givechian, K. B., et al. Identification of an immune gene expression signature associated with favorable clinical features in Treg-enriched patient tumor samples. NPJ Genom Med 3, 14 (2018).
56. Eisenhauer, E. A., et al. New response evaluation criteria in solid tumours: revised RECIST guideline (version 1.1). Eur J Cancer 45, 228-247 (2009).
57. Harrell, F. E., Califf, R. M., Pryor, D. B., Lee, K. L. & Rosati, R. A. Evaluating the Yield of Medical Tests. Jama-J Am Med Assoc 247, 2543-2546 (1982).
58. D'Agostino, R. B., Grundy, S., Sullivan, L. M., Wilson, P. & Grp, C. R. P. Validation of the Framingham Coronary Heart Disease prediction scores-Results of a multiple ethnic groups investigation. Jama-J Am Med Assoc 286, 180-187 (2001).
59. Larkin, J., et al. Five-Year Survival with Combined Nivolumab and Ipilimumab in Advanced Melanoma. N Engl J Med 381, 1535-1546 (2019).
60. R. C., Z. A SAS® Macro for Estimating Power for ROC Curves One-Sample and Two-Sample Cases. in Proceedings of the 20th SAS Users Group International Conference (SUGI) Paper 223 (1995).
61. Gubin et al. High-Dimensional Analysis Delineates Myeloid and Lymphoid Compartment remodeling during successful Immune-Checkpoint Cancer therapy. Cell, Volume 175, Issue 4, 1 Nov. 2018, Pages 1014-1030.e19

### Example 2: Liquid Biopsy of the Tumor Microenvironment for Immunotherapy Response and Toxicity Assessment

### Significance

Cancer is the second most common cause of death in the United States and immune checkpoint inhibitors are now a powerful way to treat advanced stages of disease^{4,5}. Most advanced-stage cancers will alter their tumor microenvironment (TME) by activating cell surface receptors on immune cells, such as PD-1 and CTLA4, that inhibit anti-tumor immune responses⁶⁻⁸. Immune checkpoint inhibitors (ICIs) block these receptors and transform a subset of tumor infiltrating leukocytes (TILs) in the TME into cancer-killing cells, a phenomenon that has revolutionized the field of oncology^{4,5}. Unfortunately, however, most patients do not respond to immunotherapy and experience poor outcomes as a result, in large part due to the cellular composition of their TME^{6-8,10-19}. This is because the TME can also contain cells that promote resistance to immune checkpoint blockade, or lack cells with cancer-killing properties^{4-8,10-21}. In standard clinical practice, we don't monitor the TME and thus cannot reliably identify early which patients will respond to immunotherapy²². While the tumor microenvironment directly underlies treatment response, TME analysis requires invasive biopsy¹¹, which is impractical to perform serially and can be dangerous to our patients^{23,24}. Here we will develop a liquid biopsy approach called LiquidTME based on digital cytometric analysis of bisulfite-treated cell-free DNA (cfDNA) next-generation sequencing (NGS) to overcome this.

### Developing LiquidTME

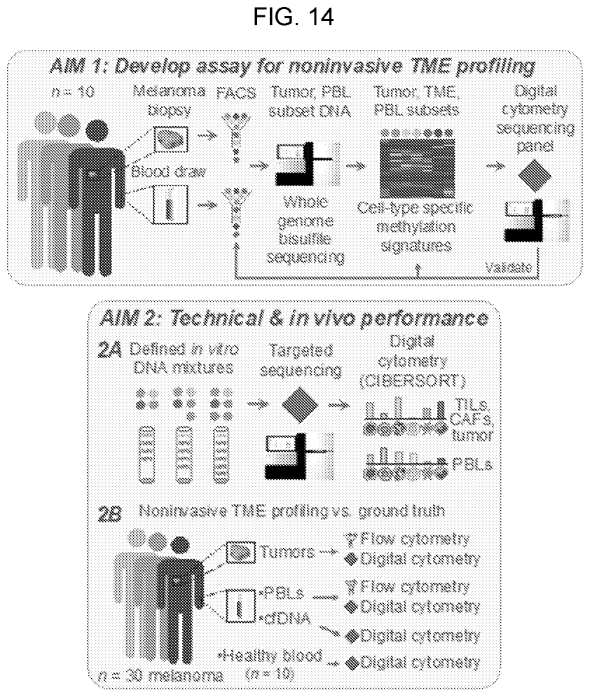

The developed liquid biopsy approach called LiquidTME can distinguish TILs from tumor cells and normal leukocytes using methylation signatures (see e.g., FIG. 14).

It was hypothesized that digital cytometry of bisulfite-treated cfDNA can robustly detect TILs, tumor cells, and peripheral blood leukocytes. We and others have shown that cell type abundances can be accurately deconvolved from bulk tissue NGS data with CIBERSORTx^{20,25-28}. Here we've developed an analogous approach to enable “digital cytometry” of bisulfite-treated cfDNA NGS data, identify and profile TILs, and distinguish them from tumor cells and normal peripheral blood leukocytes (PBLs).

### Establishing the Technical Performance of LiquidTME

Here is described establishing the technical performance of LiquidTME and determining whether it can accurately capture TIL content from cfDNA obtained from melanoma patients (see e.g., FIG. 14).

It was hypothesized that digital cytometry of cfDNA bisulfite NGS faithfully captures TIL content. Here we will apply our LiquidTME method to cfDNA isolated from melanoma patients and compare our predictions to ground truth cellular proportions from tumor flow cytometry and deconvolution of bulk tumor genomic data at matched timepoints.

### Applying LiquidTME to Predict Melanoma ICI Response

Here is described the application of LiquidTME to predict melanoma ICI response and comparison to other technologies.

It was hypothesized that digital cytometry of cfDNA bisulfite NGS enables ICI response prediction, enabling detection of molecular changes more accurately than other tumor/blood-based technologies and earlier than standard imaging. We will apply our assay pre-treatment to advanced-stage melanoma patients treated with ICIs, identify signatures of response, validate these in a held-out test set, and compare to clinical/imaging surveillance, peripheral blood TCR sequencing, tumor PDL1 proportion score, and pre-treatment tumor genomic features.

### Background

### Cell-Free DNA

Physiologic cfDNA in the blood is thought to arise from cell death²⁹⁻³². Malignant tumors also shed DNA into the circulation (ctDNA), where it can be isolated, quantitated, and sequenced²⁹⁻³⁵. Mechanisms of release of ctDNA into the bloodstream are related to tumor cell death²⁹⁻³³. The challenge with ctDNA detection is that levels in the blood plasma are low, typically comprising a minority of normal cell-free DNA molecules³². Modern NGS-based techniques have thus been developed which enable ctDNA detection as low as ˜0.01% of total cell-free DNA, low enough to detect post-treatment molecular residual disease (MRD)^{36,37}. Just as tumor cells secrete ctDNA, we hypothesized that the tumor microenvironment also sheds cell-free DNA that can be effectively measured using highly sensitive methods (FIG. 8). We refer to this new type of cell-free DNA as “circulating tumor infiltrating leukocyte DNA” or “ctilDNA”.

### Immunotherapy Response

ICIs are currently transforming cancer care and have improved the outcomes of a subset of patients with advanced cancer^{4,5,38}. Still, immunotherapy response in individual patients is unpredictable, with overall rates ranging from 1% to 60%, and most cancer types having a response rate of 5-20%³⁹. Making matters more challenging, response assessment cannot be performed reliably for ˜3 months after starting treatment because standard-of-care CT imaging cannot reliably distinguish between true progression and pseudoprogression at earlier timepoints⁴⁰⁻⁴². As this first scan may still be subject to pseudoprogression⁴⁰⁻⁴², current radiographic guidelines recommend that in cases of suspected progression, a second scan should be ordered at least one month later (˜4 months after starting immunotherapy) to provide confirmation⁴¹⁻⁴³. Despite these efforts, delayed pseudoprogression occurring after this initial period has still been described^{41,42} Previous studies showed that earlier response assessment could be performed by serial tumor biopsies analyzed by immunohistochemistry and genomics^{11,44,45}, a compelling approach but clinically impractical. It is thus critical to develop a liquid biopsy method to assess immune checkpoint inhibitor response early that can also be applied serially with ease, which is our plan here.

### Melanoma

Melanoma is the fifth most common cancer in the United States and a poster child for immunotherapy response, with objective response rates as high as ˜60% with combination ICIs⁴⁶. Despite this, clinical outcomes remain poor with a 4-year survival rate of only ˜50%⁴⁶. Cell-free DNA and ctDNA concentrations are typically elevated in advanced-stage patients, with multiple papers demonstrating the ability to assess this compartment by plasma liquid biopsy^{9,47-52}. Given poor clinical outcomes, high cfDNA content, and a clear role for immunotherapy, it is worth focusing on this cancer type for these studies.

### Bisulfite Sequencing

Bisulfite sequencing involves treatment of DNA with bisulfite to identify methylated bases, followed by NGS to identify patterns of DNA methylation. These methylation patterns can be used to identify tissue-of-origin^{53,54}. Recent publications demonstrate the utility of methylation profiling to detect tumor cell-derived cfDNAS⁵⁵⁻⁵⁷. Still, the composition of the TME has not been profiled epigenetically from cell-free DNA. We plan to bridge this gap here using a novel approach.

### Data

Molecular Profiles Distinguish TILs from PBLs

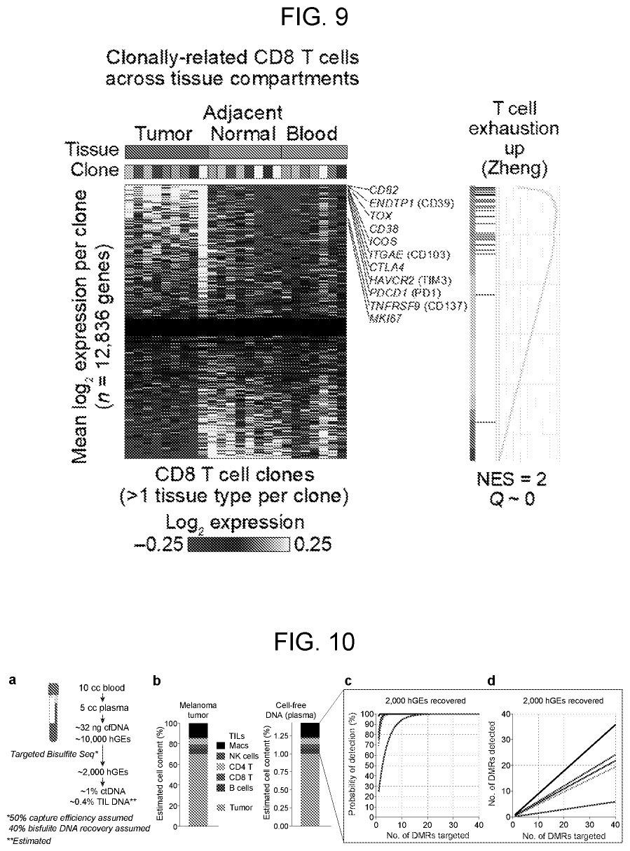

Philip et al. used ATAC-seq to demonstrate distinct epigenetic programs in tumor-specific CD8 T cells indicative of cellular dysfunctions⁵⁸. Building upon this result, we analyzed scRNA-seq data from T cells isolated from hepatocellular cancer patients (Zheng et al.⁵⁹) and identified stereotypic differences between CD8 T cells of the same clonotype found in >1 tissue compartment: tumor, adjacent normal, and/or peripheral blood (FIG. 9). Markers associated with T cell exhaustion and dysfunction⁷ (i.e., ICOS, PD1, and CTLA4) as well as those associated with tumor reactivity⁶⁰ (i.e., CD103 and CD39) were consistently upregulated in tumor CD8 T cells but were low or absent in the same clonotypes from adjacent normal and PBL compartments. This data suggests we can distinguish TILs from PBLs using epigenetics.

Mathematical Modeling of ctilDNA Detection by Plasma cfDNA Analysis

Factors underlying the detection limit of cell-free DNA applications include: (1) the number of cell-free DNA molecules that are recovered, and (2) the number of independent “reporters” in a patient's tumor that are interrogated¹. Regarding these factors, using a validated binomial model that was previously described for predicting circulating tumor DNA detection limits¹, we estimated the number of unique cell type-specific differentially methylated regions (DMRs; i.e., “reporters”) that would be needed to achieve various detection limits, considering: (1) a realistic cell-free DNA input amount (˜32 ng cell-free DNA in 1 blood collection tube¹), (2) the median circulating tumor DNA fraction in metastatic melanoma (˜1% $1) (3) estimates of TIL content in advanced melanoma tumors²⁰, (4) estimated cell-free DNA recovery rates after bisulfite conversion (20-60%⁶²), and (5) published recovery rates of cell-free DNA using hybrid capture sequencing (40-60%¹). Given ˜10,000 genome equivalents of cell-free DNA (assuming ˜32 ng cell-free DNA) and assuming an 80% DNA loss from library preparation, the modeling suggests >10 DMRs per cell type would be sufficient for TIL detection with 95% confidence (FIG. 10). Our model suggests a high chance of success as the detection limit required to track ctilDNA is within the range of what we can reliably achieve with ctDNA^{32,36,37}.

TME Signatures can be Detected in cfDNA

It was next queried whether the tumor microenvironment signal can be detected in cell-free DNA using a liquid biopsy technique. To do this, we FACS-sorted CD45+ TILs and EPCAM+ tumor cells from 3 cryopreserved colorectal cancer (CRC) tumor samples and their corresponding PBLs, and performed whole genome bisulfite sequencing. We used metilene⁶³ for differential methylated region analysis, identifying distinct DMRs between each population which we used as reporters for deconvolution. We then performed whole genome bisulfite sequencing (WGBS) of cell-free DNA from these patients using an Illumina NovaSeq S4 flow cell targeting 4050 genome-wide coverage, and queried these reporters using deconvolution by non-negative least squares regression. Strikingly using this approach even at this low sequencing depth, we were able to detect TIL signal from blood plasma in 2 of the 3 patients (FIG. 12). We also detected tumor signal in all three patients. Indicative of our method's specificity, TIL signal was not detectable in the PBL compartment. Also, as we would expect, the PBL signal was lower in tumor than in the periphery. Only TIL and tumor signals in cell-free DNA correlated positively with flow cytometry and imaging in matched tumors. We can significantly extend this work to optimize our assay, determine whether multiple TIL subsets can be quantitated in plasma, and demonstrate clinical utility.

### Application of LiquidTME to Melanoma

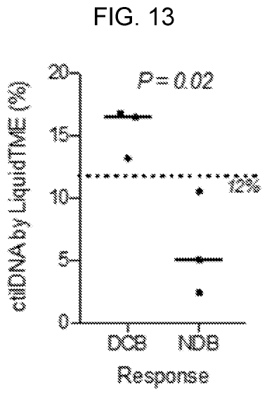

We next applied our assay to melanoma in a pilot setting. To do this, we analyzed banked pre-treatment plasma samples from 12 patients with advanced-stage melanoma with samples acquired within a month of starting immune checkpoint blockade. The response rate for this pilot cohort was 58%. We then applied a version of LiquidTME described above to each of these samples, and detected ctilDNA in 6 samples (50%) with the remaining 6 falling below the assay's limit-of-detection. Interestingly, the three patients with detectable ctilDNA patients who achieved durable clinical benefit (DCB64,6%) had significantly elevated ctilDNA levels compared to those who achieved no durable benefit (NDB) (P=0.02) (FIG. 13), with a ctilDNA cutoff of 12% perfectly classifying patients by their durable response status (FIG. 13, FIG. 4B). Kaplan-Meier analysis based on this optimal cutpoint stratified long-term survivors from short-term progressors perfectly (HR=15.3, P=0.02) (FIG. 4C). Our data supports that we can predict melanoma response to immunotherapy using LiquidTME applied to an early timepoint.

### Experimental Design and Methods

Developing a Liquid Biopsy Platform that Distinguishes TME Cells from Tumor Cells and Normal Leukocytes Using Methylation Signatures

### Defining and Validating Digital Cytometry Signatures for TILs, Tumor Cells, and PBLs

We will analyze banked viably preserved tumor and PBMC samples from 10 patients with advanced melanoma and isolate TILs, tumor cells, and PBLs by FACS. Nine major leukocyte subsets will be profiled from tumor and PBL samples: Naïve and memory CD8 T cells and CD4 T cells, NK cells, naïve and memory B cells, monocytes/macrophages, and granulocytes. We will also isolate MAGE1+ tumor cells. We will extract at least 10 ng genomic DNA from each of these samples (˜1.5 k cells/sample), including corresponding bulk tumors and PBLs, and perform WGBS. To do this, we will utilize the Zymo EZ DNA Methylation-Lightning kit for bisulfite conversion, Swift Biosciences Accel-NGS Methyl-Seq DNA kit for library preparation, and Illumina NovaSeq for 4050 coverage WGBS. We will analyze these data to identify specific signatures for each cell type using metilene⁶³ to identify DMRs and random forests, glmnet, and/or previous optimization schemes^{27,28} for feature selection. We will evaluate the discriminatory power of these signatures by applying them to bulk tumor and PBL methylation profiles from an additional 10 patients with ground truth proportions determined by flow cytometry and by bulk tissue RNA-seq deconvolution³³. These analyses will be used to establish a minimal set of ˜1,500 DMRs that discriminate melanoma tumor cells, distinct TME subsets, and PBL subsets.

Designing a DNA Capture Panel for Targeted Melanoma TME Bisulfite Sequencing We will design a capture panel that targets all DMRs identified above to maximize analytical sensitivity and to improve error tolerance^{1,66}. Other regions will be added according to their clinical or biological relevance (e.g., ICI co-inhibitory receptors) until a final size of ˜2,000 genomic intervals is achieved (˜100 bp each). We will evaluate both commercially available and published approaches for panel design (e.g., molecular-inversion probes⁵⁵).

We will (1) define TIL-, PBL- and melanoma-specific methylation signatures for the purpose of deconvolution, and (2) design an optimized sequencing panel with the genomic bandwidth to profile melanoma tumor cells, TILs, and PBLs with high analytical sensitivity.

If higher sensitivity is desired for distinguishing between distinct TIL, PBL, and tumor populations we will perform deeper WGBS (˜65) to reduce the rate of coverage dropout, expand our capture panel to include more genomic regions, profile additional patients, and/or pool cell types into broader phenotypic classes.

Establishing the Technical Performance of LiquidTME and Determine Whether it can Accurately Capture TIL Content from cfDNA Obtained from Melanoma Patients

### Assessing the Technical Performance of the Assay Using Defined In Vitro Mixtures

To evaluate the accuracy and lower limit of detection of our method, we will create a series of defined mixtures in which sonicated DNA from tumor cells, TIL, and PBL subsets (remaining from obtained above or sorted from additional patients) is added into Horizon synthetic plasma in vitro. Simulated TME content in plasma will range from 5% to <0.1% to emulate TIL content in melanoma tumors adjusted for clinically realistic ctDNA amounts^{8,9,11,17,19,44,47-52,67}. Using the panel, targeted bisulfite sequencing will be applied to DNA mixtures of 10, 20, 30, and 50 ng, and digital cytometry will be used to assess levels of each TME component. These analyses will establish performance expectations and will allow us to tune the method for maximal sensitivity and specificity.

Performing TME Profiling on cfDNA and Bulk PBMCs and Evaluate Concordance with Paired Tumors

We will analyze banked cryopreserved tumor, PBL, and plasma samples from 30 patients with melanoma. Patients underwent tumor biopsy and blood was drawn pre-treatment. A subset of patients with relapse specimens will also be assessed, enabling evaluation of changes in TME content from baseline. In parallel, we will process banked blood samples (plasma and bulk PBLs) from 10 age-matched healthy controls. We will isolate cfDNA from plasma samples and genomic DNA from tumor and PBLs. We will compare cellular abundance estimates from our platform with flow cytometry in order to (1) assess methodologic accuracy and precision and (2) determine whether cfDNA or PBL DNA better captures TIL content.

We will (1) profile TIL subsets from genomic DNA and cfDNA, (2) accurately quantify and discriminate TILs from normal PBLs in cfDNA, (3) extend our analysis in FIG. 12 to show the superiority of cfDNA over PBLs for capturing TME content, and (4) demonstrate high specificity by comparison to healthy cfDNA and PBLs.

If cell-free DNA amounts may are too low to distinguish different TIL subsets, although not anticipated, as studies have shown high ctDNA levels in advanced melanoma^{9,47-52}, we can increase input cfDNA mass and sequencing depth and refine signatures to improve detection. Separately, since tumor dissociation may distort flow cytometry²⁸, we will compare ctilDNA profiles with tumor RNA-seq deconvolution.

### Applying LiquidTME to Predict Melanoma ICI Response, Compared to Other Technologies

We have banked serial blood samples from >100 advanced-stage melanoma patients treated first-line with ICIs. Patients in parallel received standard-of-care CT imaging, and were followed for at least 1 year to determine rates of response vs. progression. Approximately half of these patients achieved durable clinical benefit while the remainder developed progressive disease. We will utilize pre-treatment plasma samples from 50 patients (randomly selected) and assess ctilDNA pre-treatment to identify characteristics corresponding with durable clinical benefit (i.e. increased ctilDNA content). We will then analyze the remaining 50 patients from the bank in order to validate the response profile learned from our test set. We will assess ROC AUC, and compare LiquidTME to PDL1 tumor proportion score, peripheral blood TCR sequencing, NGS profiles of pre-treatment tumors (i.e., “hot” vs. “cold” RNA signatures, tumor mutational burden), and CT imaging scored by RECIST 1.168. Cox regression will be performed to associate these factors with progression-free and overall survival.

### Statistical Considerations

To determine the required sample sizes of our training and validation cohorts, we assumed patients will have a response rate of 50%. Based on conservative forecasting from our data, we assumed 25% higher 1-year response rate for patients with a TIL response signature, and 25% lower response rate for patients with a TIL nonresponder signature. To achieve 90% power to reject the null hypothesis that there will be no difference in PFS between the 2 groups (alpha=0.05, two-tailed), we will need to analyze data from at least 38 patients. Additional ˜30% will be analyzed per cohort to account for attrition.

We will (1) determine a TIL profile from pre-treatment cfDNA (i.e., elevated ctilDNA content like FIG. 2B) that corresponds to durable clinical benefit to ICI treatment, (2) validate it using a held-out cohort, and (3) demonstrate more accurate response and outcomes prediction than the other technologies tested.

If sensitivity remains suboptimal, then we can implement methods to improve the analytical limit of detection such as bioinformatic background error correction¹, addition of DMRs/reporters to the capture panel, greater sequencing depth, and optimization of deconvolution through machine learning. We will also analyze early on-treatment samples (˜4 weeks on treatment) to boost the clinical sensitivity/specificity of our approach if necessary, as early on-treatment assessment is still valuable even if pre-treatment assessment is challenging.

### Innovation

This technology is a highly innovative combination of cfDNA bisulfite sequencing and digital cytometry to profile the TME in solid tumor cancer patients by liquid biopsy for the first time. This approach will help address a major unmet need: predicting ICI response early.

### REFERENCES

1. Newman, A. M., et al. Integrated digital error suppression for improved detection of circulating tumor DNA. Nat Biotechnol 34, 547-555 (2016).
2. Worm Orntoft, M. B., Jensen, S. O., Hansen, T. B., Bramsen, J. B. & Andersen, C. L. Comparative analysis of 12 different kits for bisulfite conversion of circulating cell-free DNA. Epigenetics 12, 626-636 (2017).
3. Siegel, R. L., Miller, K. D. & Jemal, A. Cancer statistics, 2019. CA Cancer J Clin 69, 7-34 (2019).
4. Postow, M. A., Callahan, M. K. & Wolchok, J. D. Immune Checkpoint Blockade in Cancer Therapy. J Clin Oncol 33, 1974-1982 (2015).
5. Ribas, A. & Wolchok, J. D. Cancer immunotherapy using checkpoint blockade. Science 359, 1350-1355 (2018).
6. Gajewski, T. F., Schreiber, H. & Fu, Y. X. Innate and adaptive immune cells in the tumor microenvironment. Nat Immunol 14, 1014-1022 (2013).
7. Thommen, D. S. & Schumacher, T. N. T Cell Dysfunction in Cancer. Cancer Cell 33, 547-562 (2018).
8. Tumeh, P. C., et al. PD-1 blockade induces responses by inhibiting adaptive immune resistance. Nature 515, 568-571 (2014).
9. Zill, O. A., et al. The Landscape of Actionable Genomic Alterations in Cell-Free Circulating Tumor DNA from 21,807 Advanced Cancer Patients. Clin Cancer Res 24, 3528-3538 (2018).
10. Thorsson, V., et al. The Immune Landscape of Cancer. Immunity 48, 812-830 e814 (2018).
11. Chen, P. L., et al. Analysis of Immune Signatures in Longitudinal Tumor Samples Yields Insight into Biomarkers of Response and Mechanisms of Resistance to Immune Checkpoint Blockade. Cancer Discov 6, 827-837 (2016).
12. Fridman, W. H., Pages, F., Sautes-Fridman, C. & Galon, J. The immune contexture in human tumours: impact on clinical outcome. Nat Rev Cancer 12, 298-306 (2012).
13. Harper, J. & Sainson, R. C. Regulation of the anti-tumour immune response by cancer-associated fibroblasts. Semin Cancer Biol 25, 69-77 (2014).
14. Kalluri, R. The biology and function of fibroblasts in cancer. Nat Rev Cancer 16, 582-598 (2016).
15. Lambrechts, D., et al. Phenotype molding of stromal cells in the lung tumor microenvironment. Nat Med 24, 1277-1289 (2018).
16. Pang, Y. L., et al. The immunosuppressive tumor microenvironment in hepatocellular carcinoma. Cancer Immunol Immunother 58, 877-886 (2009).
17. Riaz, N., et al. Tumor and Microenvironment Evolution during Immunotherapy with Nivolumab. Cell 171, 934-949 e915 (2017).
18. Spranger, S. & Gajewski, T. F. Impact of oncogenic pathways on evasion of antitumour immune responses. Nat Rev Cancer 18, 139-147 (2018).
19. Van Allen, E. M., et al. Genomic correlates of response to CTLA-4 blockade in metastatic melanoma. Science 350, 207-211 (2015).
20. Gentles, A. J., et al. The prognostic landscape of genes and infiltrating immune cells across human cancers. Nat Med 21, 938-945 (2015).
21. Mariathasan, S., et al. TGFbeta attenuates tumour response to PD-L1 blockade by contributing to exclusion of T cells. Nature 554, 544-548 (2018).
22. Kataoka, Y. & Hirano, K. Which criteria should we use to evaluate the efficacy of immune-checkpoint inhibitors? Ann Transl Med 6, 222 (2018).
23. Shyamala, K., Girish, H. C. & Murgod, S. Risk of tumor cell seeding through biopsy and aspiration cytology. J Int Soc Prev Community Dent 4, 5-11 (2014).
24. Yam, C., et al. Risk of needle-track seeding with serial ultrasound guided biopsies in triple negative breast cancer. Cancer Res 78 (2018).
25. Chakravarthy, A., et al. Pan-cancer deconvolution of tumour composition using DNA methylation. Nat Commun 9, 3220 (2018).
26. Corces, M. R., et al. Lineage-specific and single-cell chromatin accessibility charts human hematopoiesis and leukemia evolution. Nat Genet 48, 1193-1203 (2016).
27. Newman, A. M., et al. Robust enumeration of cell subsets from tissue expression profiles. Nat Methods 12, 453-457 (2015).
28. Newman, A. M., et al. Determining cell type abundance and expression from bulk tissues with digital cytometry. Nat Biotechnol (2019).
29. Schwarzenbach, H., Hoon, D. S. & Pantel, K. Cell-free nucleic acids as biomarkers in cancer patients. Nat Rev Cancer 11, 426-437 (2011).
30. Chaudhuri, A. A., Binkley, M. S., Osmundson, E. C., Alizadeh, A. A. & Diehn, M. Predicting Radiotherapy Responses and Treatment Outcomes Through Analysis of Circulating Tumor DNA. Seminars in radiation oncology 25, 305-312 (2015).
31. Wan, J. C. M., et al. Liquid biopsies come of age: towards implementation of circulating tumour DNA. Nat Rev Cancer 17, 223-238 (2017).
32. Chin, R. I., et al. Detection of Solid Tumor Molecular Residual Disease (MRD) Using Circulating Tumor DNA (ctDNA). Mol Diagn Ther 23, 311-331 (2019).
33. Stroun, M., Anker, P., Lyautey, J., Lederrey, C. & Maurice, P. A. Isolation and characterization of DNA from the plasma of cancer patients. European journal of cancer & clinical oncology 23, 707-712 (1987).
34. Corcoran, R. B. & Chabner, B. A. Application of Cell-free DNA Analysis to Cancer Treatment. N Engl J Med 379, 1754-1765 (2018).
35. Heitzer, E., Haque, I. S., Roberts, C. E. S. & Speicher, M. R. Current and future perspectives of liquid biopsies in genomics-driven oncology. Nat Rev Genet 20, 71-88 (2019).
36. Abbosh, C., Birkbak, N. J. & Swanton, C. Early stage NSCLC-challenges to implementing ctDNA-based screening and MRD detection. Nat Rev Clin Oncol 15, 577-586 (2018).
37. Chaudhuri, A. A., et al. Early Detection of Molecular Residual Disease in Localized Lung Cancer by Circulating Tumor DNA Profiling. Cancer Discov 7, 1394-1403 (2017).
38. Emens, L. A., et al. Cancer immunotherapy: Opportunities and challenges in the rapidly evolving clinical landscape. Eur J Cancer 81, 116-129 (2017).
39. Yarchoan, M., Hopkins, A. & Jaffee, E. M. Tumor Mutational Burden and Response Rate to PD-1 Inhibition. N Engl J Med 377, 2500-2501 (2017).
40. Chiou, V. L. & Burotto, M. Pseudoprogression and Immune-Related Response in Solid Tumors. J Clin Oncol 33, 3541-3543 (2015).
41. Nishino, M. Immune-related response evaluations during immune-checkpoint inhibitor therapy: establishing a “common language” for the new arena of cancer treatment. J Immunother Cancer 4, 30 (2016).
42. Hodi, F. S., et al. Evaluation of Immune-Related Response Criteria and RECIST v1.1 in Patients With Advanced Melanoma Treated With Pembrolizumab. J Clin Oncol 34, 1510-1517 (2016).
43. Wolchok, J. D., et al. Guidelines for the evaluation of immune therapy activity in solid tumors: immune-related response criteria. Clin Cancer Res 15, 7412-7420 (2009).
44. Cooper, Z. A., et al. Distinct clinical patterns and immune infiltrates are observed at time of progression on targeted therapy versus immune checkpoint blockade for melanoma. Oncoimmunology 5, e1136044 (2016).
45. Roh, W., et al. Integrated molecular analysis of tumor biopsies on sequential CTLA-4 and PD-1 blockade reveals markers of response and resistance. Sci Transl Med 9 (2017).
46. Hodi, F. S., et al. Nivolumab plus ipilimumab or nivolumab alone versus ipilimumab alone in advanced melanoma (CheckMate 067): 4-year outcomes of a multicentre, randomised, phase 3 trial. Lancet Oncol 19, 1480-1492 (2018).
47. Tsao, S. C., et al. Monitoring response to therapy in melanoma by quantifying circulating tumour DNA with droplet digital PCR for BRAF and NRAS mutations. Sci Rep 5, 11198 (2015).
48. Bettegowda, C., et al. Detection of circulating tumor DNA in early- and late-stage human malignancies. Sci Transl Med 6, 224ra224 (2014).
49. Lee, J. H., et al. Circulating tumour DNA predicts response to anti-PD1 antibodies in metastatic melanoma. Ann Oncol 28, 1130-1136 (2017).
50. Lipson, E. J., et al. Circulating tumor DNA analysis as a real-time method for monitoring tumor burden in melanoma patients undergoing treatment with immune checkpoint blockade. J Immunother Cancer 2, 42 (2014).
51. Xi, L., et al. Circulating Tumor DNA as an Early Indicator of Response to T-cell Transfer Immunotherapy in Metastatic Melanoma. Clin Cancer Res 22, 5480-5486 (2016).
52. Cabel, L., et al. Circulating tumor DNA changes for early monitoring of anti-PD1 immunotherapy: a proof-of-concept study. Ann Oncol 28, 1996-2001 (2017).
53. Roadmap Epigenomics, C., et al. Integrative analysis of 111 reference human epigenomes. Nature 518, 317-330 (2015).
54. Feinberg, A. P. Phenotypic plasticity and the epigenetics of human disease. Nature 447, 433-440 (2007).
55. Xu, R. H., et al. Circulating tumour DNA methylation markers for diagnosis and prognosis of hepatocellular carcinoma. Nat Mater 16, 1155-1161 (2017).
56. Moss, J., et al. Comprehensive human cell-type methylation atlas reveals origins of circulating cell-free DNA in health and disease. Nat Commun 9, 5068 (2018).
57. Shen, S. Y., et al. Sensitive tumour detection and classification using plasma cell-free DNA methylomes. Nature 563, 579-583 (2018).
58. Philip, M., et al. Chromatin states define tumour-specific T cell dysfunction and reprogramming. Nature 545, 452-456 (2017).
59. Zheng, C., et al. Landscape of Infiltrating T Cells in Liver Cancer Revealed by Single-Cell Sequencing. Cell 169, 1342-1356 e1316 (2017).
60. Duhen, T., et al. Co-expression of CD39 and CD103 identifies tumor-reactive CD8 T cells in human solid tumors. Nat Commun 9, 2724 (2018).
61. Zill, O. A., et al. The Landscape of Actionable Genomic Alterations in Cell-Free Circulating Tumor DNA from 21,807 Advanced Cancer Patients. Clinical Cancer Research 24, 3528-3538 (2018).
62. Worm Ørntoft, M.-B., Jensen, S. Ø., Hansen, T. B., Bramsen, J. B. & Andersen, C. L. Comparative analysis of 12 different kits for bisulfite conversion of circulating cell-free DNA. Epigenetics 12, 626-636 (2017).
63. Juhling, F., et al. metilene: fast and sensitive calling of differentially methylated regions from bisulfite sequencing data. Genome Res 26, 256-262 (2016).
64. Rizvi, H., et al. Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol 36, 633-641 (2018).
65. Rizvi, N. A., et al. Cancer immunology. Mutational landscape determines sensitivity to PD-1 blockade in non-small cell lung cancer. Science 348, 124-128 (2015).
66. Newman, A. M., et al. An ultrasensitive method for quantitating circulating tumor DNA with broad patient coverage. Nat Med 20, 548-554 (2014).
67. Tirosh, I., et al. Dissecting the multicellular ecosystem of metastatic melanoma by single-cell RNA-seq. Science 352, 189-196 (2016).
68. Eisenhauer, E. A., et al. New response evaluation criteria in solid tumours: revised RECIST guideline (version 1.1). Eur J Cancer 45, 228-247 (2009).
69. Chaudhuri, A. A., et al. Circulating tumor DNA quantitation for early response assessment of immune checkpoint inhibitors for lung cancer. in American Radium Society Annual Meeting (Orlando, FL, 2018).
70. Azad, T. D., et al. Circulating Tumor DNA Analysis for Detection of Minimal Residual Disease After Chemoradiotherapy for Localized Esophageal Cancer. Gastroenterology (2019).

### Example 3: Developing a Liquid Biopsy Approach for Tumor Microenvironment Profiling

### Problem

Immune checkpoint inhibitors have transformed modern cancer treatment as the only therapeutic in years to provide durable remission and significant survival benefit across many cancer types. Despite their success, most patients do not respond to these drugs, there is a serious risk of immune-related toxicity, and we are unable to reliably predict response or toxicity early. The key to unlocking the full potential of immune checkpoint inhibitors is through understanding the tumor microenvironment (TME). However, the only way to analyze the TME is through invasive biopsy which is impractical to perform serially and can cause harm to the patient.

### Solution

Here, we disclose the development and testing of a liquid biopsy method for tumor microenvironment profiling based on next-generation methylation sequencing of cell-free DNA. This method, which we call LiquidTME, will be developed in the context of colorectal and lung cancers (two of the most common cancers worldwide) but will be directly extensible to nearly any malignancy. If successful, our approach will enable tumor microenvironment analysis through a simple blood test, which should have a direct clinical impact by enabling earlier and more precise assessment of the thousands of cancer patients being treated with immunotherapy.

Cancer is the second most common cause of death in the United States¹ and immune checkpoint inhibitors are now a powerful way to treat advanced stages of disease^{2,3}. Most advanced-stage cancers will alter their tumor microenvironment (TME) by activating cell surface receptors on immune cells, such as PD-1 and CTLA4, that inhibit anti-tumor immune responses⁴⁻⁶. Immune checkpoint inhibitors block these receptors and transform a subset of tumor infiltrating leukocytes (TILs) in the TME into cancer-killing cells, a phenomenon that has revolutionized the field of oncology^{2,3}.

Unfortunately, however, most patients do not respond to immunotherapy and experience poor outcomes as a result, in large part due to the cellular composition of their TME⁴⁻¹⁶. This is because the TME can also contain cells that promote resistance to immune checkpoint blockade, or lack cells with cancer-killing properties²⁻¹⁸. In standard clinical practice, we don't monitor the TME and thus cannot reliably identify early which patients will respond to immunotherapy¹⁹. There is also a serious risk of immune-related adverse events²⁰, with examples of fatalities reported in the literature^{21,22}. While the tumor microenvironment directly underlies treatment response and likely plays an important role in toxicity as well²³, TME analysis requires invasive biopsy⁷, which is impractical to perform serially and can be dangerous to our patients^{24,25}. Here we describe a non-invasive liquid biopsy approach called LiquidTME to overcome this challenge.

Our approach for a TME liquid biopsy will take advantage of the fact that tumors continually shed DNA into the circulation, where it can be isolated as cell-free circulating tumor DNA (ctDNA)²⁶⁻³⁰. Mechanisms of release of ctDNA into the bloodstream are related to tumor cell death²⁶⁻³⁰. The challenge with ctDNA detection is that levels in the blood plasma are low, typically comprising <1% of normal cell-free DNA molecules²⁶. Modern NGS-based techniques have thus been developed which enable ctDNA detection as low as ˜0.01% of total cell-free DNA, low enough to detect post-treatment molecular residual disease (MRD)^{31,32}. Just as tumor cells secrete ctDNA, we hypothesized that the tumor microenvironment also sheds cell-free DNA that can be effectively measured using highly sensitive methods (FIG. 8). We refer to this new type of cell-free DNA as “circulating tumor infiltrating leukocyte DNA” or “ctilDNA”.

Disclosed here is an ultra-sensitive approach to detect ctilDNA by tracking highly specific epigenomic markers on DNA rather than tumor mutations. The epigenome is comprised of chemical compounds bound to the DNA molecule that direct which parts of the genome are turned on or off³³. Each cell type has a unique epigenomic signature³³ which we can profile by analyzing the methylation pattern on DNA using a method called bisulfite sequencing^{34,35}. We will use these epigenomic signatures to distinguish cell types through machine learning based cellular deconvolution, similar conceptually to CIBERSORT^{36,37}, but applied to the minuscule levels of ctilDNA present in blood plasma. To support this, we performed a mathematical modeling exercise using this approach (FIG. 10). Our model suggests a high chance of success as the detection limit required to track ctilDNA is within the range of what we reliably achieve with ctDNA^{26,31,32}.

Importantly, tumor infiltrating leukocytes (TILs) differ from their normal peripheral blood leukocyte (PBL) counterparts as shown by recent single cell RNA sequencing studies of lung and breast tumors^{11,41,42}. Demonstrating that this difference is also seen in the epigenome, Philips et al. utilized ATAC-Seq to demonstrate distinct epigenomic programs in tumor-specific CD8 T cells indicative of cellular dysfunction⁴³. To significantly extend upon this result, we re-analyzed published single cell RNA sequencing (scRNA-seq) data from T cells isolated from hepatocellular cancer patients (Zheng et al.⁴⁴) and clearly observe stereotypic differences between tumor infiltrating CD8 T cells and their normal counterparts (from both adjacent normal tissue and PBLs) (FIG. 10). We also did this analysis at the clonotype level and strikingly, CD8 T cells with the same T cell receptor (originating from the same precursor) still show striking epigenomic differences between tumor and normal, indicating their ultimate site of tumor vs. normal tissue/blood residence is a major determinant of their expression signature despite their clonal genomic identity. Markers associated with T cell exhaustion and dysfunction⁵ (i.e., ICOS, PD1 and CTLA4) as well as those associated with tumor reactivity⁴⁵ (i.e., CD103 and CD39) were consistently upregulated in the tumor CD8 T cells but were low or absent in the same clonotypes from other compartments. This data is compelling and suggests we can exploit these differences between TILs and normal PBLs to identify TIL signatures from cell-free DNA even though the majority of cell-free DNA arises from normal PBLs.

This technology is based on the premise that ultra-sensitive detection and profiling of TME-derived ctilDNA will enable early and precise cancer treatment response and toxicity assessment. Our approach will utilize machine learning to combine data from methylation sequencing studies (e.g., ENCODE⁴⁶, BLUEPRINT⁴⁷, NIH Roadmap Epigenomics Project³³) with our own data that we generate through methylation sequencing of patient samples, with innovative technical methods to sensitively and specifically detect individual TME cellular subsets (i.e., CD8 T cells, CD4 T cells, NK cells, B cells, monocytes/macrophages, cancer-associated fibroblasts) from cell-free DNA. This technology is a noninvasive TME profiling assay that we will apply to cancers, such as lung and colorectal cancers, which should easily extend to all common cancer types. Therefore, the potential impact of our work is immense and, if successful, our assay could become a routine laboratory test that is ordered for thousands of patients annually. Serial ctilDNA monitoring will finally provide clinicians with a real-time window into the inner workings of the tumor microenvironment and enable them to toggle their treatments accordingly (i.e., pivot early to alternate treatment if a patient is unlikely to respond or is likely to experience a severe toxicity).

### Clinical Relevance

We wish to re-emphasize the potential clinical importance of this research. Immune checkpoint inhibitors are transforming cancer care and have improved the outcomes of a multitude of patients with advanced-stage cancer^{2,3}. In my field of practice (lung cancer), immunotherapy has improved survival dramatically in patients with both locally advanced and advanced disease⁴⁹⁻⁵², enabling many to live longer than ever thought possible. Still, immunotherapy response in individual patients is unpredictable, with overall rates ranging between 1% to 50%, and most cancer types having a response rate of 5-20%⁵³. Making matters more challenging, response assessment cannot be performed reliably for ˜3 months after starting treatment because standard-of-care CT imaging cannot distinguish between true progression and pseudoprogression at earlier timepoints⁵⁴⁻⁵⁶. As this first scan may still be subject to pseudoprogression⁵⁴⁻⁵⁶, current radiographic guidelines recommend that in cases of suspected progression, a second scan should be ordered at least one month later (˜4 months after starting immunotherapy) to provide confirmation⁵⁵⁻⁵⁷. Despite these efforts, delayed pseudoprogression occurring after this initial period have still been described^{55,56} Recent studies have shown that earlier response assessment could be performed by serial tumor biopsies analyzed by immunohistochemistry and genomics^{7,58,59}, an approach that is compelling but clinically impractical. It is thus critical to develop a liquid biopsy method to assess immune checkpoint inhibitor response early that can also be applied serially with ease, which is what is presently disclosed here. Given the broad importance of the tumor microenvironment, the technology we develop will be applicable to other clinical and research settings as well.

On the flip-side of immunotherapy response is toxicity²⁰. Rates of severe toxicity requiring hospitalization are ˜60% in patients treated with combination immune checkpoint inhibitors (anti-CTLA4 and anti-PD1), and ˜25% in those treated with a single agent^{60,61}. Unfortunately multiple instances of death resulting from immune checkpoint blockade have also been documented^{21,22}. In a large meta-analysis of 613 patients who experienced fatal immune checkpoint blockade-related toxicity, the median time to death after starting treatment was only 14.5 days in those receiving combination immune checkpoint inhibitors, and 40 days in those receiving either anti-PD1 or anti-CTLA4 alone²¹, highlighting that biomarkers must be developed to predict these as early as possible. While higher toxicity rates are associated with certain mechanisms of action (i.e. anti-CTLA4 vs. anti-PD1)^{60,61}, the precise pathophysiology underlying these severe immune-related adverse events is unknown, with translational studies showing that multiple immune pathways may be involved²⁰. There is some suggestion that B cells play an important role in toxicity⁶², and a recent report in Nature Medicine implicated oligoclonal expansion of CD4 T cells targeting an EBV-specific and an EBV-like domain in a case of fatal encephalitis²². Using LiquidTME, we will be able to profile cell-free DNA from TILs and circulating leukocytes in a single assay, allowing us to track a diverse repertoire of immune cell dynamics before and during treatment. As such, we hypothesized that we will gain new insights into the biology of toxicity, with implications for clinicians to consider alternative treatment in patients deemed high-risk for toxicity based on the results of our test. Our method could thus be used to identify and track immune-related toxicity from immunotherapy and potentially other modalities as well.

Disclosed herein, is a novel method for detecting tumor microenvironment-derived DNA in cell-free DNA called LiquidTME. LiquidTME entails purifying pre-determined genomic regions that are highly enriched for DMRs which identify and distinguish tumor microenvironmental cellular subsets from their normal counterparts. LiquidTME will be ultra-sensitive and directly applicable to cancer patients, with the most immediate clinical role being the early prediction of immunotherapy response and toxicity. In describing the experimental plan for developing LiquidTME we will first detail the technical development of the method and then describe experiments to evaluate its clinical utility. Thus, this technology can result in the delivery of an optimized method for profiling the tumor microenvironment noninvasively that has passed initial clinical validation applied to immunotherapy patients. Here, LiquidTME is developed in the context of CRC and NSCLC.

We have chosen to focus on colorectal cancer (CRC) and non-small cell lung cancer (NSCLC) because these are among the most common causes of cancer and of cancer death worldwide⁶³. Additionally, I am a practicing radiation oncologist who specializes in the treatment of lung and gastrointestinal cancers, and thus have clinical expertise in this arena and ready access to specimens. I believe our LiquidTME test will be extensible to other cancer types as well, perhaps requiring only slight optimization. Focusing on NSCLC and CRC for now will enable us to develop and test the method in a defined clinical setting first, and in a setting where my clinical expertise and access to specimens is greatest.

### LiquidTME Proof-of-Concept Experiments

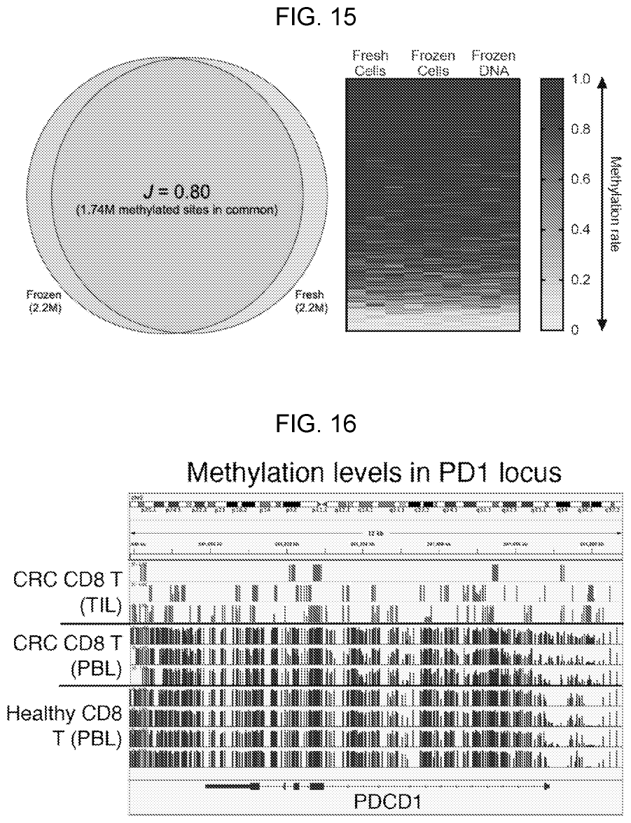

We began with the mathematical modeling experiment in FIG. 10, and the TIL vs. normal scRNA-seq analysis in FIG. 9. We next asked a practical question for methodological development-Does freezing affect the cellular epigenetic methylation profile? To answer this, we performed whole genome bisulfite sequencing (WGBS) of 9 healthy peripheral blood leukocyte samples from a healthy donor, with all sample preparation performed fresh (without freezing) on 3 samples, DNA frozen for 3 samples, and the cells cryopreserved prior to all further processing for the remaining 3 samples. Following WGBS on all 9 samples, we observed no major differences in global methylation patterns (FIG. 15), suggesting that cryo-banking cells or DNA does not introduce epigenetic artifacts, consistent with prior literature⁶⁴.

We next generated proof-of-concept data that methylation signatures differ between individual TIL subsets and their normal counterparts. To do this, we isolated sorted CD8 T cell subsets from 3 cryopreserved CRC patients' tumors as well as peripheral blood CD8 T cells from these same patients, then performed whole genome bisulfite sequencing followed by sequence alignment and methylation analysis. We then performed differential methylated region analysis using Metilene⁶⁵ and compared methylation levels in these samples, as well as against publicly available healthy donor CD8 T cells available through the BLUEPRINT⁴⁷ project. We observed that methylation levels were diminished in genes associated with T cell exhaustion/dysfunction, including ICOS, PDCD1 and CTLA4 in CD8 TILs (corroborating our scRNA-Seq analysis in FIG. 9). This is shown in FIG. 16 for the PDCD1 gene locus.

We next queried whether the tumor microenvironment signal can be detected in cell-free DNA using a liquid biopsy technique. To do this, we began by FACS-sorting CD45+ TILs and EPCAM+ tumor cells from 3 cryopreserved CRC tumor samples and their corresponding peripheral blood leukocytes, and performed whole genome bisulfite sequencing. We used Metilene⁶⁵ for differential methylated region analysis, identifying distinct DMRs between each population, then queried these in cell-free DNA using deconvolution via non-negative least squares regression. We performed whole genome bisulfite sequencing of cell-free DNA using an Illumina NovaSeq S4 flow cell targeting 4050 genome-wide coverage, and strikingly even at this low sequencing depth, we were able to detect the TIL signal from blood plasma in 2 of the 3 patients (FIG. 12). We also detected tumor signal in all three patients. Indicative of our method's specificity, this TIL signal was not detectable in the peripheral blood cell compartment. Also, as we would expect, the peripheral blood cell signal was lower in tumor than in the periphery. Only TIL and tumor signals in cell-free DNA correlated positively with flow cytometry and imaging in matched tumors. We now plan to significantly extend this initial work to optimize our assay, determine whether multiple TME subsets can be quantitated in plasma, and demonstrate clinical utility in the setting of immunotherapy.

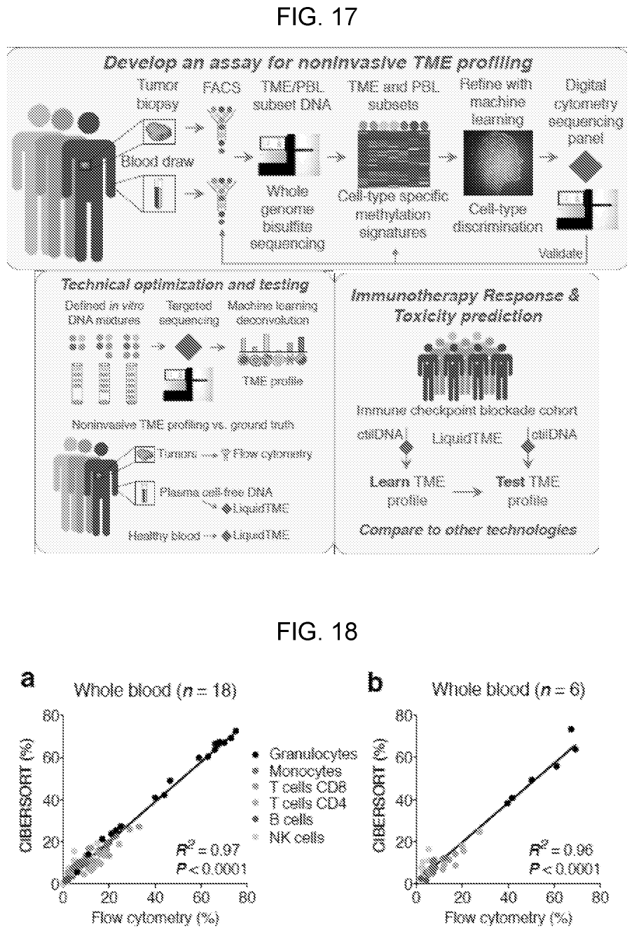

To develop LiquidTME for noninvasive TME profiling, we will follow the roadmap outlined in FIG. 17. For LiquidTME to function robustly, it will require distinct input signatures derived from our cell types of interest. We will thus begin by FACS-purifying viably preserved tumor and peripheral blood leukocyte samples from 10 patients with advanced CRC or NSCLC and isolate major leukocyte subsets including naïve and memory CD8 and CD4 T cells, NK and NK T cells, naïve and memory B cells, myeloid derived suppressor cells (MDSCs), monocytes/macrophages, and granulocytes. We will also isolate cancer-associated fibroblasts (CAFs) as they have been reported to promote an immunosuppressive tumor microenvironment^{9,10}, as well as EPCAM+ tumor cells. We will extract at least 10 ng genomic DNA from each of these samples (˜1.5 k cells/sample), including corresponding bulk tumors and plasma-depleted whole blood. To prepare samples for bisulfite sequencing, we will utilize the Zymo EZ DNA Methylation-Lightning kit for bisulfite conversion followed by the Swift Biosciences Accel-NGS Methyl-Seq DNA kit for library preparation, then sequence our samples on an Illumina NovaSeq using the S4 flow cell, aiming for 4050 genomic coverage. Following sequencing alignment and the determination of methylated sites using the BISCUIT⁶⁶ software suite and quality-control using in-house scripts, we will apply Metilene⁶⁵ for differential methylated region (DMR) analysis. In this way, we will identify specific methylation signatures corresponding to each cell type which will allow us to distinguish each TME subset from one another and from normal peripheral blood leukocytes.

By leveraging machine learning feature selection approaches, including random forests and elastic net, we will identify the DMRs most likely to enable sharp distinction between cell types (FIG. 17). We will incorporate these distinguishing DMRs into a sequencing panel (e.g., utilizing molecular-inversion probes) that can discriminate tumor cells, TME subsets and PBL subsets while achieving much greater sequencing depth (aiming for 2,000× de-duplicated depth as per FIG. 10) than WGBS (which is typically ≤40×). This is reasonable to aim for as a depth of 2,000× is typical of targeted hybrid capture based ctDNA detection methods^{26,38,67,68}, and not cost-prohibitive because the sequencing space will be limited to a small fraction of the genome^{26,29,30}.

We will next optimize our approach and validate it in blood plasma (FIG. 17). To do this, we will apply LiquidTME to pre-defined DNA mixtures derived from TME subsets and peripheral blood cells (sheared to simulate the size of cell-free DNA^{69,70}). To simulate ctilDNA in plasma, these mixtures will contain between 4% and 0.04% TME content to emulate clinically realistic levels ranging within 10-fold of our estimate in FIG. 10. We will investigate more sophisticated machine learning based deconvolution strategies in order to infer the relative percentages of each cell type within our simulated TME mixtures. We expect a high likelihood of success, even though we expect the ctilDNA signal to be low and admixed with a high background of normal leukocyte DNA. Moreover, FIG. 12 shows we were able to detect the TME signal within cell-free DNA without the major technical innovations discussed here. LiquidTME can be validated clinically by applying the assay to plasma samples from patients with advanced-stage CRC and NSCLC (FIG. 12). We also have cryopreserved banked tumor samples from these patients from the same timepoints. We will assess the accuracy and precision of our method in these clinical samples, as compared to flow cytometry on the dissociated tumor. In addition to flow cytometry, we will also perform CIBERSORT^{36,37} on the tumor samples as tissue dissociation can lead to variable loss of fragile cell types and distort flow cytometry results by favoring cell types that fit through the filter and instrument pores³⁷. To clinically validate performance, we will compare concordance between our method applied to cell-free DNA and gold-standard tumor analysis.

Before proceeding to clinical practice evaluation of LiquidTME, we explore several physical properties of ctilDNA. ctilDNA has not been explored and we are defining it for the first time here. Having established our method, we will utilize this opportunity to analyze biophysical properties that might make ctilDNA unique from its ctDNA and normal cell-free DNA counterparts. First, we will explore whether ctilDNA has a unique size distribution as has been observed for ctDNA^{69,70}. A unique size distribution would allow us to enrich for ctilDNA upfront using bead-based cell-free DNA size selection as groups are now doing for ctDNA⁷¹. Second, we will explore whether ctilDNA is enriched in exosomes. Exosomes are microvesicles that are present in plasma and can contain nucleic acids⁷². To test whether TME-derived cell-free DNA is enriched inside or outside of exosomes we will perform fractionation of plasma using previously described methods⁷³ and will sequence exosome-enriched and -depleted fractions. Finally, while our understanding of ctDNA and the data shown in FIG. 18 suggest significant enrichment of ctilDNA in the cell-free vs. cellular compartment of blood, we will confirm this and compare our results to gold-standard tumor assessment. These studies will increase our biophysical understanding of ctilDNA and could lead to methods to enrich for it.

To establish the clinical utility of LiquidTME, we will test it in a cohort of patients treated with immune checkpoint blockade for whom we have response and toxicity data (FIG. 12). Since last year we have been collecting samples from CRC and NSCLC patients treated at Washington University, and have a 700-box-80° freezer in my lab dedicated to the effort. Samples are processed immediately after collection using a standardized protocol and are split into aliquots for cryostorage.

To test the utility of LiquidTME, we will apply it to advanced-stage NSCLC and CRC patients being treated with immunotherapy (FIG. 12). Immune checkpoint blockade-based approaches have become standard-of-care for advanced-stage NSCLC and MSI-high CRC patients, with response rates of ˜30-50% overall^{51,52,74,75} Unfortunately for the majority of patients who are nonresponders⁵³, it can take months to confirm this lack of response through CT imaging (first scan at ˜3 months, with a confirmatory scan ˜1 month later)^{19,54,55,57,61}. To begin to address this issue, we will apply LiquidTME to ˜50 patients treated with immune checkpoint blockade. We will apply our assay before any treatment and again 2-3 weeks into treatment (with chemotherapy cycle 2 blood draw). We will correlate results from our assay to ultimate clinical response to therapy. We expect to demonstrate increased CD8 T, NK, and NK T TILs, as well as decreased immunosuppressive macrophages, MDSCs and CAFs in responders vs. progressors^{4,14}. We will confirm this “response TME profile” by flow cytometry and CIBERSORT^{36,37} using pre-treatment biopsy samples (and on-treatment biopsies when available). Also, we will compare our technology to other recent and emerging methods such as peripheral blood T cell receptor sequencing⁷⁶, “hot” vs. “cold” tumor RNA expression signatures⁷⁷, tumor proportion score of PD-L178, and tumor mutational burden^{53,79}. The hope is that our technology will demonstrate robustness and outperform these other methods. It will of course be important to validate our results in an independent external cohort, which we plan to do with help from our clinical collaborators.

Finally, we will determine if we can predict severe toxicities from immune checkpoint blockade using our LiquidTME method (FIG. 12). Unfortunately, there are no biomarkers for immune-related adverse events in clinical use²⁰. This is an important issue in patients with thoracic and gastrointestinal malignancies, as pneumonitis and colitis are among the most common adverse events^{20,21}, which can compound when treatment is delivered with other modalities concurrently^{80,81}. To begin to address this, we will use the same ˜50 patient cohort described above at the same timepoints (pre-treatment and 2-3 weeks on-treatment). Based on published literature⁶⁰, I expect ˜25% of these patients to experience severe toxicity. We will categorize these adverse events, and correlate toxicity type and incidence with TIL and circulating leukocyte dynamics from our LiquidTME assay. B cells and CD4 T cells have been recently suggested to be related to immune-related adverse events^{22,62}. We will determine if this is the case using our assay. As before, we will compare our TME predictions to pre-treatment tumor biopsies (and on-treatment when available) analyzed by flow cytometry and CIBERSORT^{36,37}. It will be critical to confirm our results externally, which we plan to do. If successfully validated, these results will enable us to more safely deliver immunotherapy by anticipating severe toxicity before its clinical occurrence.

Our inability to accurately predict immunotherapy response or toxicity early is one of the most challenging problems in clinical cancer research. This technology can solve this problem through the development of LiquidTME represents a highly innovative approach. LiquidTME could revolutionize immunotherapy response and toxicity assessment in two ways. First, it could serve as a primary assessment modality that provides precise data to the clinician at a timepoint when imaging and clinical assessment has been shown to be inadequate. Secondly, it could be used to serially track patients and supplement equivocal assessments from our standard clinical modalities, helping to distinguish borderline response from progression and predict the severity of potential symptomatic toxicity. Our work here can be generalized even more broadly, as noninvasive TME assessment can find utility in multiple research and clinical settings.

This technology tracks a previously undescribed entity (ctilDNA), to do this robustly and comprehensively, and to apply our technology to a clinical challenge of utmost importance in the field of oncology.

### Innovation

The presently described technology is exceptionally innovative since it is on the topic of a new and previously undescribed component of cell-free DNA, which arises from the tumor microenvironment, and we disclose a new technical method in order to profile and track it in the blood. Our method presents a potential solution to one of the most significant problems to arise in modern oncology, namely the prediction of which patients will respond to immunotherapy and which patients will be affected by severe toxicities from immunotherapy. If successful, LiquidTME will be a groundbreaking advance in immunotherapy response and toxicity assessment, having a palpable clinical impact. This would revolutionize oncologic practice by enabling us to more precisely select and monitor our patients and potentially impact the lives of thousands of individuals annually. Moreover, by robustly profiling the tumor microenvironment noninvasively, our work here should generalize to nearly any cancer type and anti-cancer therapy, opening the door to routine and noninvasive tumor microenvironment assessment in both research and clinical settings.

A variety of methods can be employed to increase sensitivity. First, we can expand the targeted sequencing panel to include more differentially methylated regions. We can also sequence to greater depth in order to more sensitively detect ctilDNA^{28,32}. The main drawbacks of these optimizations are that sequencing costs will increase. However, sequencing costs have been plummeting and are expected to continue to decrease⁸². To increase sensitivity further, we can decrease the number of TME cellular subsets we are tracking; for example, we may restrict ourselves to just B cells, CD8 T cells, CD4 T cells, NK cells, and monocytes/macrophages rather than all 12 TME cell types described above. If successful, we expect this stripped-down approach to still be clinically highly meaningful as it will include the broad categories typically assessed by standard flow cytometry⁸³.

### REFERENCES

1. Siegel, R. L., et al. C A Cancer J Clin 69, (2019).
2. Postow, M. A., et al. J Clin Oncol 33, (2015).
3. Ribas, A., et al. Science 359, (2018).
4. Gajewski, T. F., et al. Nat Immunol 14, (2013).
5. Thommen, D. S., et al. Cancer Cell 33, (2018).
6. Tumeh, P. C., et al. Nature 515, (2014).
7. Chen, P. L., et al. Cancer Discov 6, (2016).
8. Fridman, W. H., et al. Nat Rev Cancer 12, (2012).
9. Harper, J., et al. Semin Cancer Biol 25, (2014).
10. Kalluri, R. Nat Rev Cancer 16, (2016).
11. Lambrechts, D., et al. Nat Med 24, (2018).
12. Pang, Y. L., et al. Cancer Immunol Immunother 58, (2009).
13. Riaz, N., et al. Cell 171, (2017).
14. Spranger, S., et al. Nat Rev Cancer 18, (2018).
15. Thorsson, V., et al. Immunity 48, (2018).
16. Van Allen, E. M., et al. Science 350, (2015).
17. Gentles, A. J., et al. Nat Med 21, (2015).
18. Mariathasan, S., et al. Nature 554, (2018).
19. Kataoka, Y., et al. Ann Transl Med 6, (2018).
20. Postow, M. A., et al. N Engl J Med 378, (2018).
21. Wang, D. Y., et al. JAMA Oncol 4, (2018).
22. Johnson, D. B., et al. Nat Med 25, (2019).
23. Sanmamed, M. F., et al. Cell 176, (2019).
24. Shyamala, K., et al. J Int Soc Prev Community Dent 4, (2014).
25. Yam, C., et al. Cancer Res 78, (2018).
26. Chin, R. I., et al. Mol Diagn Ther 23, (2019).
27. Schwarzenbach, H., et al. Nat Rev Cancer 11, (2011).
28. Stroun, M., et al. European journal of cancer & clinical oncology 23, (1987).
29. Wan, J. C. M., et al. Nat Rev Cancer 17, (2017).
30. Chaudhuri, A. A., et al. Seminars in radiation oncology 25, (2015).
31. Abbosh, C., et al. Nat Rev Clin Oncol 15, (2018).
32. Chaudhuri, A. A., et al. Cancer Discov 7, (2017).
33. Bernstein, B. E., et al. Nat Biotechnol 28, (2010).
34. Deng, J., et al. Nat Biotechnol 27, (2009).
35. Gu, H., et al. Nat Protoc 6, (2011).
36. Newman, A. M., et al. Nat Methods 12, (2015).
37. Newman, A. M., et al. Nat Biotechnol, (2019).
38. Newman, A. M., et al. Nat Biotechnol 34, (2016).
39. Worm Orntoft, M. B., et al. Epigenetics 12, (2017).
40. Zill, O. A., et al. Clin Cancer Res 24, (2018).
41. Zilionis, R., et al. Immunity 50, (2019).
42. Azizi, E., et al. Cell 174, (2018).
43. Philip, M., et al. Nature 545, (2017).
44. Zheng, C., et al. Cell 169, (2017).
45. Duhen, T., et al. Nat Commun 9, (2018).
46. Consortium, E. P. Science 306, (2004).
47. Martens, J. H., et al. Haematologica 98, (2013).
48. Shen, S. Y., et al. Nature 563, (2018).
49. Antonia, S. J., et al. N Engl J Med 379, (2018).
50. Antonia, S. J., et al. N Engl J Med 377, (2017).
51. Gandhi, L., et al. N Engl J Med 378, (2018).
52. Reck, M., et al. N Engl J Med 375, (2016).
53. Yarchoan, M., et al. N Engl J Med 377, (2017).
54. Chiou, V. L., et al. J Clin Oncol 33, (2015).
55. Nishino, M. J Immunother Cancer 4, (2016).
56. Hodi, F. S., et al. J Clin Oncol 34, (2016).
57. Wolchok, J. D., et al. Clin Cancer Res 15, (2009).
58. Cooper, Z. A., et al. Oncoimmunology 5, (2016).
59. Roh, W., et al. Sci Transl Med 9, (2017).
60. Haanen, J., et al. Ann Oncol 28, (2017).
61. Hodi, F. S., et al. Lancet Oncol 19, (2018).
62. Das, R., et al. J Clin Invest 128, (2018).
63. Ferlay, J., et al. Int J Cancer 127, (2010).
64. Li, Y., et al. Epigenomics 10, (2018).
65. Juhling, F., et al. Genome Res 26, (2016).
66. Zhou, W., et al. github.com/zwdzwd/biscuit, (2018).
67. Newman, A. M., et al. Nat Med 20, (2014).
68. Odegaard, J. I., et al. Clin Cancer Res 24, (2018).
69. Mouliere, F., et al. PLOS One 6, (2011).
70. Underhill, H. R., et al. PLOS Genet 12, (2016).
71. Mouliere, F., et al. Sci Transl Med 10, (2018).
72. Pegtel, D. M., et al. Annu Rev Biochem 88, (2019).
73. Greening, D. W., et al. Methods Mol Biol 1295, (2015).
74. Le, D. T., et al. Journal of Clinical Oncology 36, (2018).
75. Overman, M. J., et al. Journal of Clinical Oncology 36, (2018).
76. Zaretsky, J. M., et al. Journal of Clinical Oncology 33, (2015).
77. Givechian, K. B., et al. NPJ Genom Med 3, (2018).
78. Roach, C., et al. Appl Immunohistochem Mol Morphol 24, (2016).
79. Goodman, A. M., et al. Mol Cancer Ther 16, (2017).
80. Minor, D. R., et al. Pigment Cell Melanoma Res 28, (2015).
81. Oshima, Y., et al. JAMA Oncol 4, (2018).
82. McCombie, W. R., et al. Cold Spring Harb Perspect Med, (2018).
83. Jimenez Vera, E., et al. PLOS One 14, (2019).
84. Baltimore, D., et al. Nat Immunol 9, (2008).
85. O'Connell, R. M., et al. Nat Rev Immunol 10, (2010).
86. O'Connell, R. M., et al. Proc Natl Acad Sci USA 106, (2009).
87. O'Connell, R. M., et al. J Exp Med 205, (2008).
88. O'Connell, R. M., et al. Proc Natl Acad Sci USA 107, (2010).
89. Chaudhuri, A. A., et al. Proc Natl Acad Sci USA 109, (2012).
90. So, A. Y., et al. Blood 124, (2014).
91. Barnell, E., et al. Gastroenterology, (2019).
92. Chaudhuri, A. A., et al. Lung Cancer 89, (2015).

### Example 4: Noninvasive Til Quantitation

The following example describes the development of an ultrasensitive framework for profiling tumor infiltrating leukocytes using cell-free DNA methylation profiles and evaluate the technical performance of noninvasive digital cytometry for profiling TILs in vitro and from patients with metastatic melanoma.

Tumor infiltrating leukocytes (TILs) play critical roles in tumor growth, cancer progression, and patient outcomes. While techniques for characterizing TIL composition (e.g., flow cytometry, immunohistochemistry) have generated profound insights into cancer biology and medicine, they generally require tumor biopsy or resection procedures that are invasive, associated with morbidity, and may not account for geographic tumor heterogeneity. There are currently no reliable methods for assessing TIL composition noninvasively.

Liquid biopsies are an emerging class of techniques for noninvasive tumor profiling based on cell-free DNA, which is continually shed into the circulation from normal and malignant cells. Despite the potential of cell-free DNA to enable safe, noninvasive assessment of diverse physiological states over serial time points, there is currently no liquid biopsy method available for monitoring TIL composition. A genomics platform applied to cell-free DNA can enable noninvasive profiling of TIL subsets to precisely profile the tumor microenvironment. This can be achieved via bisulfite-treated next-generation sequencing of plasma-derived cell-free DNA, followed by deconvolution of cell composition from methylation signatures, which we will apply to metastatic melanoma as a proof-of-principle. We hypothesized that our method for “noninvasive digital cytometry” will enable accurate, biopsy-free monitoring of the tumor microenvironment without being limited to (1) small combinations of preselected marker genes (as flow cytometry is), (2) T/B cell receptor variable regions (as VDJ profiling is), or (3) viable single cells (as single cell RNA sequencing is). Importantly, the kinetics of cell-free DNA release from TILs is unknown and whether methylation signatures can quantitatively capture specific non-malignant tumor cell types from cell-free DNA has not yet been established. The following experiments were designed to address these technical questions and a novel assay for safe, high resolution profiling of TIL dynamics in cancer patients.

### Developing an Ultrasensitive Framework for Profiling Tumor Infiltrating Leukocytes Using Cell-Free DNA Methylation Profiles

It was hypothesized that DNA methylation signatures can robustly distinguish TILs from other cell types and enable their highly sensitive quantitation from small quantities of DNA.

A. Define cell type-specific methylation signatures that distinguish major TIL subsets from normal peripheral blood leukocytes and non-hematopoietic cells. Here we will apply whole genome bisulfite sequencing to sorted melanoma TIL subsets, malignant melanocytes, stromal cells, and normal peripheral blood leukocytes, to define TIL-specific methylation sites. We will then develop and validate a computational framework to infer the proportions of individual cell types from admixtures of methylated DNA.

B. Design and optimize the performance of a targeted bisulfite sequencing panel to profile TILs from clinically realistic DNA input amounts. We will devise analytical methods to design a cost-effective capture sequencing panel that targets multiple TIL-specific genomic reporters while maximizing sensitivity from small DNA quantities (e.g., cfDNA amount obtained in a single blood collection tube).

Evaluating the Technical Performance of Noninvasive Digital Cytometry for Profiling TILs In Vitro and from Patients with Metastatic Melanoma

It was hypothesized that noninvasive digital cytometry faithfully captures TIL content in defined in vitro mixtures and in cell-free DNA from melanoma patients.

A. Assess the technical performance of noninvasive digital cytometry using defined in vitro mixtures. To evaluate the accuracy and lower limit of detection of our method, we will create a series of defined mixtures in which sonicated DNA from tumor leukocyte subsets is added into cell-free DNA from healthy donors in vitro. Total leukocyte content will emulate immune levels in melanoma tumors adjusted for clinically realistic circulating tumor DNA amounts. Using the panel described above, targeted bisulfite sequencing will be applied to these DNA mixtures over a range of input quantities, and noninvasive digital cytometry will be used to assess TIL content. We will thus establish performance expectations and tune our method to maximize sensitivity and specificity.

B. Perform noninvasive TIL profiling in melanoma patients and evaluate concordance with paired tumors. For in vivo validation, we will analyze banked viably preserved tumor, plasma, and peripheral blood mononuclear cell (PBMC) samples (from matched time-points) from 30 patients with metastatic melanoma. In parallel, we will process banked blood samples (plasma and PBMCs) from 10 age-matched healthy controls (who should have no TILs present). We will compare TIL predictions by our method to orthogonal measures of TIL content in paired tumors (e.g., by flow cytometry), and will compare methylation signatures from cell-free DNA to cellular DNA (PBMCs) to determine which compartment better captures known TIL composition.

### Research Approach

### Significance

Tumor infiltrating leukocytes (TILs) play critical roles in tumor growth, cancer progression, and patient outcomes (1-8). While recent advances in immuno-oncology are revolutionizing cancer treatment, patient responses to existing and emerging immunotherapies are often heterogeneous and effective predictive biomarkers are lacking (9-12). For example, there are currently no biomarkers with high sensitivity/specificity for predicting early which patients are likely to benefit from immune checkpoint inhibitors (ICIs) and which are not (11-13). Although a number of powerful techniques for characterizing TIL composition are available (e.g., flow cytometry, immunohistochemistry, CyTOF, single cell RNA sequencing), they generally require tumor biopsy or resection procedures that are invasive (14), associated with morbidity (15), and may not account for geographic tumor heterogeneity (16, 17). As a result, due to limited tumor availability, most analyses of human TIL composition are restricted to a single snapshot of tumor heterogeneity obtained from a single time point.

This barrier has left major gaps in our understanding of TIL dynamics, hampering our ability to leverage these cells for the development of more effective biomarkers and therapies.

The presently described technology can be a new technology for noninvasive TIL quantitation. The ability to noninvasively monitor TIL composition would provide an attractive solution to the above problem in both research and clinical settings. However, there are currently no reliable methods for biopsy-free TIL assessment. Previous studies of peripheral blood leukocytes (PBLs) in cancer patients have identified subpopulations that resemble those found in tumors and that have prognostic/predictive potential (18, 19); however, the cell type marker profiles employed in these studies are unlikely to be TIL-specific and the extent to which these cells truly capture tumor immune composition is unclear (20). Separately, while highly specific T cell receptor (TCR) clonotypes from tumors can be found and tracked in the peripheral blood (21, 22), this approach (1) provides a limited view of TIL heterogeneity and (2) cannot distinguish between tumor-derived and normal T cells without highly biased clonotype representation or prior knowledge of tumor-specific TCRs.

Over the last few years, many groups, including ours, have developed and validated techniques for the noninvasive detection of tumor burden and tumor genotypes using plasma-derived circulating tumor DNA, a form of cell-free DNA released into the peripheral blood where it can be isolated, quantitated and sequenced (23-26). Physiologic cell-free DNA in the blood is mostly derived from non-malignant cells, and is thought to arise from cell death due to necrosis, apoptosis, phagocytosis, and possibly also active secretion (24-26). This raises the possibility that TIL-derived cell-free DNA may be detectable in plasma and could serve as a noninvasive readout of TIL heterogeneity. Although multiple studies have profiled and tracked circulating tumor DNA using PCR and next generation sequencing (NGS)-based methods and demonstrated high sensitivity (24, 27-30), the degree to which cell-free DNA captures TIL biology in solid tumors has not yet been explored. Here we describe the novel methodology, which will demonstrate that TIL DNA can be detected and quantitated within the plasma of cancer patients. This technology will have implications for noninvasive TIL diagnostics.

The development of an assay for noninvasive TIL profiling could revolutionize our understanding of tumor immunology, with applications for the discovery of improved biomarkers for diverse anti-cancer therapies. For example, ICIs are currently transforming cancer care, and have improved the outcomes of a subset of patients with advanced cancer, giving them remarkable therapeutic responses and allowing a subset of these responders to achieve long-term survival (9, 31-33). ICI response rates for different cancers range from 1% to 50% (34), with response rates affected by multiple factors, including tumor PDL1 expression, tumor mutation burden, neoantigen load, and tumor histology (34-37). Standard-of-care for assessing ICI response is serial CT imaging that begins 2-3 months after initiating immunotherapy (38), and is assessed by RECIST 1.1 (39) or iRECIST (40) criteria. CT imaging is typically performed no earlier than 2-3 months after treatment initiation due to delayed radiographic responses and concern for pseudoprogression at earlier time points (13, 38, 41). This approach will allow investigators to explore methods of earlier immunotherapy response assessment in order to pivot sooner to more effective treatment modalities for progressors, who comprise the majority of patients.

Toward this end, we will benchmark the technical performance of our assay on patients with advanced melanoma, a ‘poster child’ for solid tumor immunotherapy (42). Although some melanoma patients show durable anti-tumor T cell responses to ICIs, many fail to respond, and the treatment is often linked to immune-related adverse events, such as colitis, pneumonitis, hepatitis, and endocrine disorders (43, 44). Cell-free DNA and circulating tumor DNA concentrations are typically elevated in metastatic melanoma patients (29, 45), indicating there is sufficient material to assess this compartment noninvasively. Given the heterogeneous clinical outcomes, high cell-free DNA content, and established role for immunotherapy, we believe it is worthwhile to focus on melanoma for this technical study.

### Innovation

This technology will provide a platform for the following innovations:

First, cell-free DNA harbors epigenetic signatures that are informative for tissue-of-origin, including methylated cytosines in CpG dinucleotides, which have distinct lineage-specific patterns and can be profiled using bisulfite sequencing (46). The Lo group showed that genome-wide bisulfite sequencing enabled tissue-of-origin identification of plasma-derived cell-free DNA in pregnant women, organ transplant patients, and hepatocellular carcinoma patients (47). Zhang and colleagues applied whole genome bisulfite sequencing with linkage disequilibrium principles to identify tightly coupled CpG sites, which they called methylation haplotype blocks (48). Methylation haplotype blocks were more accurate at discriminating between tissue-specific methylation patterns than conventional methylation metrics, and enabled cancer tissue of origin identification from cell-free DNA from patients with different malignancies (48). Despite these results, the composition of the tumor immune microenvironment has not been profiled by methylation signatures in cell-free DNA. This technology can yield a novel framework that addresses this gap using targeted bisulfite sequencing.

Second, flow cytometry and immunohistochemistry are commonly used to dissect tissue cellular composition. However, both approaches generally rely on small combinations of preselected marker genes, limiting the number of cell types that can be simultaneously interrogated. Although single cell RNA sequencing has emerged as a powerful technology for defining novel cell subsets (49), it is currently impractical for large-scale analyses. To complement these methods and to facilitate cellular profiling of large patient cohorts, we previously developed CIBERSORT, an “in silico flow cytometry” method for enumerating cell composition from bulk tissue gene expression profiles (50). When evaluated on fresh, frozen, and fixed specimens, CIBERSORT outperformed previous computational methods and compared favorably to flow cytometry and immunohistochemistry (3, 50). Moreover, in a pan-cancer analysis of ˜6,000 human tumors, CIBERSORT revealed important new associations between TILs and clinical outcomes (3). This method can be adapted for the deconvolution of cell-free DNA bisulfite sequencing data, allowing us to determine the proportions of distinct TIL subsets from cell type-specific methylation profiles identified in cell-free DNA.

Third, this approach can help address a major unmet need: monitoring TIL dynamics at high resolution over serial time points to advance biomarker discovery and precision cancer medicine.

### Approach

The experiments described here are to develop and experimentally evaluate the new platform for noninvasive profiling of TILs from melanoma patients. This research can involve an innovative combination of experimental and computational approaches, including tools developed by the investigative team, to build a novel genomics platform for profiling and decoding TIL-derived methylation signatures identified from plasma-derived cell-free DNA molecules. The research plan is schematically depicted in FIG. 14. Here, we describe the application of whole genome bisulfite sequencing to define cell type-specific methylation signatures of major TIL subsets from primary patient tumors. Also described is the design and optimization of a next generation sequencing panel and corresponding computational framework to profile TIL-specific methylation sites from clinically practical amounts of plasma-derived cell-free DNA. We describe the testing of our assay for “noninvasive digital cytometry” by benchmarking its performance on both defined DNA mixtures and DNA isolated from tumors, peripheral blood, and plasma obtained from patients with metastatic melanoma, with ground truth TIL proportions in paired tumors determined by flow cytometry. In both sets of experiments, we can leverage banked and de-identified melanoma biospecimens available to us from the Yale SPORE in Skin cancer (YSPORE). These specimens, which include melanoma biopsies, plasma samples, and peripheral blood leukocyte samples, have been collected with the informed signed consent of participants according to Health Insurance Portability and Accountability Act (HIPAA) regulations with a Human Investigative Committee protocol.

### Developing an Ultrasensitive Framework for Profiling Tumor Infiltrating Leukocytes Using Cell-Free DNA Methylation Profiles

Defining Cell Type-Specific Methylation Signatures that Distinguish Major TIL Subsets from Normal Peripheral Blood Leukocytes and Non-Hematopoietic Cells

### Rationale

High-throughput methylation profiling has revealed extraordinary insights into the epigenetic landscape of distinct tissue types and cellular lineages, including normal immune subsets (53). However, to our knowledge, a comparative analysis of genome-wide methylation signatures in major melanoma TIL subsets versus their normal peripheral blood counterparts has not yet been described. To successfully identify and quantify TIL subsets using methylation profiles identified by bisulfite sequencing, it will be critical to first characterize genome-wide patterns of differentially methylated CpG dinucleotides in melanoma TILs, melanoma and healthy PBL subsets, and non-hematopoietic cells.

### Methods and Data

We will analyze banked viably preserved tumor and peripheral blood mononuclear cell (PBMC) samples from 5 patients with metastatic melanoma and isolate TILs, tumor cells, stromal elements, and PBLs by fluorescence activated cell sorting (FACS). PBLs from 5 age-matched healthy non-pregnant controls (who should have no TILs present) will also be assessed (obtained as described above). Six major leukocyte subsets will be profiled from PBL and tumor samples: CD8 T cells, CD4 T cells, NK cells, B cells, monocytes/macrophages, and granulocyte/myeloid-derived suppressor cells (MDSCs). We will extract at least 100 ng genomic DNA from each of these samples (˜10 k cells/sample), including corresponding bulk tumors and PBLs, and perform methylation profiling by whole genome bisulfite sequencing (WGBS), targeting 4050 coverage per sample with 225M 150 bp×2 reads on an Illumina NovaSeq. Importantly, WGBS has been shown to achieve better CpG coverage than reduced representation bisulfite sequencing, an alternate technique that uses restriction enzymes to enrich for CpG sites (54). WGBS will allow us to interrogate CpG sites at single nucleotide resolution across the entire genome and maximize the number of discriminatory markers that are detectable. As a quality control step, we will profile and compare methylation profiles from 3 cancer cell lines with publicly available WGBS data (55). We plan to evaluate two commercially available kits for WGBS, as described in above. Reads will be mapped to the genome and processed to identify methylation sites, as previously described (56, 57). Samples obtained from the same human donor will be verified by evaluating the concordance of germline SNPs (58).

In order to identify differentially methylated regions (DMRs) that improve TIL-specific quantification and error tolerance, we will apply a previously described linkage equilibrium-based approach to identify methylation haplotype blocks (48) (regions with multiple methylated CpGs within ˜200 contiguous bases; cell-free DNA molecules are highly stereotyped in length and are ˜170 bp (27, 28)). To improve marker specificity, we will omit from further consideration any genomic regions corresponding to haplotype blocks that are significantly differentially methylated/expressed on non-hematopoietic tissues, cell types, and melanomas using data from the NIH roadmap epigenomics project, ENCODE, BLUEPRINT, and WGBS data generated in this study. Next, we will analyze the remaining haplotype blocks to identify highly specific signatures for each cell type using our previously described approach (50), but tailored for methylation data. Using CIBERSORT (50), we will evaluate the discriminatory power of these signatures by applying them to bulk tissue methylation profiles with ground truth proportions determined by FACS. To assess the generalizability of leukocyte signatures, artificial mixtures containing publicly available DNA methylation profiles from normal leukocyte populations (59-63) will also be assessed. These analyses will be used to establish a minimal set of DMRs that maximally discriminate melanoma tumors and leukocyte subsets, including TIL and PBL populations.

As a proof-of-principle, we trained a CIBERSORT signature matrix to distinguish major PBL subsets profiled on Infinium HumanMethylation450K BeadChip arrays (64). Applied to whole blood methylation profiles generated by two groups (65, 66), we observed highly significant agreement with flow cytometry-determined proportions (FIG. 18). Additionally, given the strong concordance between methylchip arrays and WGBS (67), bisulfite sequencing should perform similarly if not better owing to higher resolution coverage of CpGs.

Finally, we will compare deconvolution performance between hyper- and hypo-methylated regions by in silico simulation to determine which of the two events, if any, should be prioritized in our panel design.

Given numerous reports of differences in the phenotypic states of TILs, normal adjacent tissues, and normal peripheral blood leukocytes (20, 68-71), we will identify many significant TIL subset-specific methylation blocks. Moreover, the identified WGBS methylation profiles will be made available as a community resource in order to promote further research into TIL-specific epigenetics. Separately, given promising data (FIG. 18), robust TIL deconvolution from admixed methylation profiles are expected.

It is possible that 4050 coverage may be inadequate to robustly identify single and/or bi-allelic methylation events. If so, we will perform additional sequencing to target 65 coverage. Should specific TIL subsets be indistinguishable from normal leukocytes, we will consider eliminating them from further analysis or pooling them into broader lineages.

Design and Optimize the Performance of a Targeted Bisulfite Sequencing Panel to Profile TILs from Clinically Realistic DNA Input Amounts.

### Rationale

Several commercially available bisulfite sequencing kits are compatible with low quantities of input DNA (e.g., cell-free DNA amounts that are obtainable in a single blood collection tube (28)). Nevertheless, achieving highly sensitive TIL cell-free DNA profiling at a low cost will require the design of a custom capture panel. Here is described the design of a targeted sequencing panel that covers multiple TIL-specific genomic reporters to maximize analytical sensitivity and to improve error tolerance (27, 28, 51).

### Methods and Data

To develop the assay, we will evaluate both commercially available and published approaches for panel design (e.g., NimbleGen SeqCap Epi Choice Probes S versus molecular-inversion probes (72)) and bisulfite sequencing (e.g., Zymo EZ DNA Methylation-Lightning Kit, Swift Biosciences Accel-NGS Methyl-Seq DNA) to determine tradeoffs between cost, DNA recovery rates, and bisulfite conversion efficiency.

Three key factors underlie the detection limit of cell-free DNA applications: (1) the number of cell-free DNA molecules that are recovered, (2) the number of independent “reporters” in a patient's tumor that are interrogated, and (3) technical background (27, 28). Regarding the first two factors, using a validated binomial model that we previously described for predicting circulating tumor DNA detection limits (27, 28), we estimated the number of unique cell type-specific differentially methylated regions (DMRs; i.e., “reporters”) that would be needed to achieve various detection limits, considering: (1) a realistic cell-free DNA input amount (˜32 ng cell-free DNA in 1 blood collection tube (28)), (2) the median circulating tumor DNA fraction in metastatic melanoma (˜1% (45)) (3) estimates of TIL content in advanced melanoma tumors (3, 72, 73), (4) estimated cell-free DNA recovery rates after bisulfite conversion (20-60% (74)), and (5) published recovery rates of cell-free DNA using hybrid capture sequencing (40-60% (28)). Given ˜10,000 genome equivalents of cell-free DNA (assuming ˜32 ng cell-free DNA) and assuming an 80% DNA loss from library preparation, the modeling suggests >10 DMRs per cell type would be sufficient for TIL detection with 95% confidence (FIG. 10). Moreover, only 12 DMRs per cell type would be needed to achieve a detection limit of ˜0.01% with a probability of 0.9. As we expect to cover tens to hundreds of DMRs per cell type, this should allow the theoretical recovery of multiple DMRs per cell type for detection limits as low as 0.01%. This is easily within the scope of ultra-deep targeted sequencing, per our previous work (27, 28). Moreover, although the specific set of DMRs per cell type will be unique, deconvolution will be used to resolve DMRs that are shared by >1 cell type. Regarding the third factor, bisulfite conversation efficiency and the intrinsic error rate of NGS may confound analytical sensitivity.

The former is reported to be high for many kits (>99% (74)), but will need to be confirmed. We have previously shown that capture-based NGS allows for the detection of circulating tumor DNA down to 0.02% fractional abundance without the use of unique molecular identifiers (UMIs) (27). We will leverage methylation haplotype blocks with multiple expected CpGs per read to correct errors in a manner analogous to error-tolerant DNA barcode sequences (75).

To build the panel, we will first identify cell type-specific DMRs within haplotype blocks that optimize deconvolution performance, as described herein. We will then review the 147,888 methylation haplotype blocks published by Guo and colleagues (48) to identify any additional methyl haplotype blocks that co-segregate with the obtained signatures for inclusion in the panel. Other regions will be added according to their clinical or biological relevance (e.g., ICI co-inhibitory receptors), until a final size of ˜200 kb is achieved (2,000 genomic intervals of ˜100 bp each).

We will (1) define TIL-, PBL- and melanoma tumor-specific methylation signatures for the purpose of deconvolution, and (2) design an optimized targeted hybrid-capture panel with the genomic bandwidth to profile TIL and PBL subsets.

It is possible that our capture panel will be insufficiently sensitive for distinguishing between distinct leukocyte and tumor populations. If this is the case, we can redesign the panel to relax our criteria for methylation haplotype blocks. This will allow us to consider DMRs with a lower density of clustered CpGs, which could identify additional discriminatory markers that improve performance.

Separately, if the error rate of bisulfite sequencing proves to be too high for profiling TIL-derived cell-free DNA below 0.1% fractional abundance, we will consider designing custom sequencing adapters with bisulfite-tolerant UMIs.

Evaluate the Technical Performance of Noninvasive Digital Cytometry for Profiling TILs In Vitro and from Patients with Metastatic Melanoma

### Assessing the Technical Performance of Noninvasive Digital Cytometry Using Defined In Vitro Mixtures.

Described here is the assessment of the technical performance of noninvasive digital cytometry using defined in vitro mixtures.

### Rationale

To evaluate the accuracy and lower limit of detection of our method, it can be important to establish initial performance expectations in a controlled in vitro titration series. This will help us tune our method to maximize sensitivity and specificity.

### Experimental Methods

We will create a series of defined mixtures in which sonicated DNA from tumor leukocyte subsets (remaining from above or sorted from 2 additional patients) is added into cell-free DNA from healthy control subjects in vitro (obtained as described here). Total leukocyte content will range from 5% down to <0.01% in order to emulate typical immune levels in metastatic melanoma tumors (3, 72, 73) adjusted for clinically realistic circulating tumor DNA amounts (27-30, 45, 51). Using the panel from above, targeted bisulfite sequencing will be applied to DNA mixtures of 10, 20, 30, and 50 ng, and deconvolution will be used to assess TIL content.

We expect to be able to noninvasively profile leukocyte populations and distinguish TILs from non-TILs by performing bisulfite sequencing of defined genomic and cell-free DNA admixtures.

Performing Noninvasive TIL Profiling in Melanoma Patients and Evaluate Concordance with Paired Tumors.

### Rationale

To assess whether noninvasive TIL profiling will have utility in vivo, it will be important to compare estimated TIL composition in the plasma of melanoma patients against orthogonal measures of TIL content in paired tumors (e.g., by flow cytometry). In addition, we will compare methylation signatures from cell-free DNA to cellular DNA (PBMCs) to determine which compartment better captures known TIL composition. These data can be useful for establishing baseline values for power calculations and dedicated biomarker studies.

### Experimental Methods

We will analyze banked viably preserved tumor, plasma, and PBL samples from 30 patients with advanced melanoma. Patients will match regional demographics, and no deliberate attempts to exclude certain genders/sexes or minority groups will be made. Patients will have undergone tumor biopsy and blood draw pre-treatment. A subset of patients with relapse specimens will also be assessed, enabling evaluation of changes in TIL content from baseline. In parallel, we will process banked whole blood samples (plasma and PBLs) from 10 age-matched healthy non-pregnant donors (who should have no TILs present) obtained from a local blood bank without regard to demographic features or certain genders/sexes. DMRs with high background in healthy cell-free DNA will be omitted from further analysis, per our previous work (28).

We will isolate cell-free DNA from plasma samples and genomic DNA from tumor and PBL samples, perform bisulfite conversion, perform targeted sequencing using the panel described herein, then apply NGS and deconvolution using techniques described herein. Cell-free DNA will be extracted from ˜5 ml of plasma using the QiaAmp Circulating Nucleic Acid Kit according to the manufacturer's instructions, and stored at −80° C. Following isolation, DNA will be quantified by Qubit dsDNA High Sensitivity Kit (Life Technologies) and Bioanalyzer (Agilent), and inspected for expected fragment length distribution and yield. As input, we will target a median of 32 ng of cell-free DNA per sample and 100 ng of tumor or PBL DNA for library preparation with the KAPA LTP Library Prep Kit (Kapa Biosystems). High-throughput sequencing will be performed on an Illumina HiSeq 4000 or NovaSeq 6000 to target a median non-deduplicated depth of ˜10,000×. Samples obtained from the same human donor will be verified by evaluating the concordance of germline SNPs (58).

In parallel, we will perform flow cytometry on tumor and PBL samples to assess relative fractions of each leukocyte population. We will compare our deconvolution results from each compartment to flow cytometry of tumors to (1) assess methodology accuracy and precision and (2) determine whether cell-free DNA or PBL genomic DNA better captures the composition of the tumor immune microenvironment.

We will (1) noninvasively profile leukocyte populations by performing bisulfite sequencing of cell-free DNA, (2) accurately quantify and discriminate TILs from normal leukocyte populations in the cell-free DNA compartment, (3) show the superiority of cell-free DNA over PBLs for capturing TIL content, and (4) demonstrate high methodological specificity of TIL detection by comparison to healthy donor-derived cell-free DNA and PBLs.

Not expected, but cell-free DNA concentrations may be too low for deconvolution of different TIL and tumor populations. This is not anticipated as being a major issue, as studies have shown high circulating tumor DNA concentrations in metastatic melanoma patients, which is sufficient for NGS-based methylation profiling. However, the biology and kinetics of cell-free DNA from TILs is unknown. If necessary, we will increase the number of input cell-free DNA genome equivalents and amount of sequencing, and will attempt to refine the signatures obtained from above to improve detection, including expanding our sequencing panel to including more methylation reporters, and possibly extending to whole genome bisulfite sequencing. If these approaches are still unsuccessful, we can focus on the peripheral blood cellular compartment (rather than cell-free DNA), to profile TILs that are in the circulation.

### REFERENCES

1. Prognostic value of tumor infiltrating lymphocytes in triple-negative breast cancers from two phase III randomized adjuvant breast cancer trials: ECOG 2197 and ECOG 1199. Adams S, Gray R J, Demaria S, Goldstein L, Perez E A, Shulman L N, Martino S, Wang M, Jones V E, Saphner T J, Wolff A C, Wood W C, Davidson N E, Sledge G W, Sparano J A, Badve S S. J Clin Oncol. 2014 Sep. 20;32 (27): 2959-66. PMID: 25071121.
2. Type, density, and location of immune cells within human colorectal tumors predict clinical outcome. Galon J, Costes A, Sanchez-Cabo F, Kirilovsky A, Mlecnik B, Lagorce-Pages C, Tosolini M, Camus M, Berger A, Wind P, Zinzindohoue F, Bruneval P, Cugnenc P H, Trajanoski Z, Fridman W H, Pages F. Science. 2006 Sep. 29;313 (5795): 1960-4. PMID: 17008531.
3. The prognostic landscape of genes and infiltrating immune cells across human cancers. Gentles A J, Newman A M, Liu C L, Bratman S V, Feng W, Kim D, Nair V S, Xu Y, Khuong A, Hoang C D, Diehn M, West R B, Plevritis S K, Alizadeh A A. Nat Med. 2015 August;21 (8): 938-45. PMID: 26193342.
4. Accessories to the crime: functions of cells recruited to the tumor microenvironment. Hanahan D, Coussens L M. Cancer cell. 2012 Mar. 20;21 (3): 309-22. PMID: 22439926.
5. Influence of tumour micro-environment heterogeneity on therapeutic response. Junttila M R, de Sauvage F J. Nature. 2013 Sep. 19;501 (7467): 346-54. PMID: 24048067.
6. The tumor microenvironment and Immunoscore are critical determinants of dissemination to distant metastasis. Mlecnik B, Bindea G, Kirilovsky A, Angell H K, Obenauf A C, Tosolini M, Church S E, Maby P, Vasaturo A, Angelova M, Fredriksen T, Mauger S, Waldner M, Berger A, Speicher M R, Pages F, Valge-Archer V, Galon J. Sci Transl Med. 2016 Feb. 24;8 (327): 327ra26. PMID: 26912905.
7. PD-1 blockade induces responses by inhibiting adaptive immune resistance. Tumeh P C, Harview C L, Yearley J H, Shintaku I P, Taylor E J, Robert L, Chmielowski B, Spasic M, Henry G, Ciobanu V, West A N, Carmona M, Kivork C, Seja E, Cherry G, Gutierrez A J, Grogan T R, Mateus C, Tomasic G, Glaspy J A, Emerson R O, Robins H, Pierce R H, Elashoff D A, Robert C, Ribas A. Nature. 2014 Nov. 27;515 (7528): 568-71. PMID: 25428505.
8. Molecular and genetic properties of tumors associated with local immune cytolytic activity. Rooney M S, Shukla S A, Wu C J, Getz G, Hacohen N. Cell. 2015 Jan. 15;160 (1-2): 48-61. PMID: 25594174.
9. Cancer immunotherapy: Opportunities and challenges in the rapidly evolving clinical landscape. Emens L A, Ascierto P A, Darcy P K, Demaria S, Eggermont A M M, Redmond W L, Seliger B, Marincola F M. Eur J Cancer. 2017 August;81:116-29. PMID: 28623775.
10. Cancer immunotherapy using checkpoint blockade. Ribas A, Wolchok J D. Science. 2018;359 (6382): 1350-5.
11. Predictive biomarkers for checkpoint inhibitor-based immunotherapy. Gibney G T, Weiner L M, Atkins M B. The Lancet Oncology. 2016 2016 Dec. 1; 17 (12): e542-e51.
12. Progress and challenges of predictive biomarkers of anti PD-1/PD-L1 immunotherapy: A systematic review. Teng F, Meng X, Kong L, Yu J. Cancer Letters. 2018 Feb. 1;414:166-73.
13. Guidelines for the evaluation of immune therapy activity in solid tumors: immune-related response criteria. Wolchok J D, Hoos A, O'Day S, Weber J S, Hamid O, Lebbe C, Maio M, Binder M, Bohnsack O, Nichol G, Humphrey R, Hodi F S. Clin Cancer Res. 2009 Dec. 1; 15 (23): 7412-20. PMID: 19934295.
14 Analysis of Immune Signatures in Longitudinal Tumor Samples Yields Insight into Biomarkers of Response and Mechanisms of Resistance to Immune Checkpoint Blockade. Chen P-L, Roh W, Reuben A, Cooper Z A, Spencer C N, Prieto P A, Miller J P, Bassett R L, Gopalakrishnan V, Wani K, De Macedo M P, Austin-Breneman J L, Jiang H, Chang Q, Reddy S M, Chen W-S, Tetzlaff M T, Broaddus R J, Davies M A, Gershenwald J E, Haydu L, Lazar A J, Patel S P, Hwu P, Hwu W-J, Diab A, Glitza I C, Woodman S E, Vence L M, Wistuba I I, Amaria R N, Kwong L N, Prieto V, Davis R E, Ma W, Overwijk W W, Sharpe A H, Hu J, Futreal P A, Blando J, Sharma P, Allison J P, Chin L, Wargo J A. Cancer Discovery. 2016.
15. Liver biopsy. Rockey D C, Caldwell S H, Goodman Z D, Nelson R C, Smith A D, American Association for the Study of Liver D. Hepatology. 2009 March; 49 (3): 1017-44. PMID: 19243014.
16. Phylogenetic ctDNA analysis depicts early-stage lung cancer evolution. Abbosh C, Birkbak N J, Wilson G A, Jamal-Hanjani M, Constantin T, Salari R, Le Quesne J, Moore D A, Veeriah S, Rosenthal R, Marafioti T, Kirkizlar E, Watkins T B K, McGranahan N, Ward S, Martinson L, Riley J, Fraioli F, Al Bakir M, Gronroos E, Zambrana F, Endozo R, Bi W L, Fennessy F M, Sponer N, Johnson D, Laycock J, Shafi S, Czyzewska-Khan J, Rowan A, Chambers T, Matthews N, Turajlic S, Hiley C, Lee S M, Forster M D, Ahmad T, Falzon M, Borg E, Lawrence D, Hayward M, Kolvekar S, Panagiotopoulos N, Janes S M, Thakrar R, Ahmed A, Blackhall F, Summers Y, Hafez D, Naik A, Ganguly A, Kareht S, Shah R, Joseph L, Marie Quinn A, Crosbie P A, Naidu B, Middleton G, Langman G, Trotter S, Nicolson M, Remmen H, Kerr K, Chetty M, Gomersall L, Fennell D A, Nakas A, Rathinam S, Anand G, Khan S, Russell P, Ezhil V, Ismail B, Irvin-Sellers M, Prakash V, Lester J F, Kornaszewska M, Attanoos R, Adams H, Davies H, Oukrif D, Akarca A U, Hartley J A, Lowe H L, Lock S, lles N, Bell H, Ngai Y, Elgar G, Szallasi Z, Schwarz R F, Herrero J, Stewart A, Quezada S A, Peggs K S, Van Loo P, Dive C, Lin C J, Rabinowitz M, Aerts H, Hackshaw A, Shaw J A, Zimmermann B G, consortium T R, consortium P, Swanton C. Nature. 2017 Apr. 26;545 (7655): 446-51. PMID: 28445469.
17. Tracking the Evolution of Non-Small-Cell Lung Cancer. Jamal-Hanjani M, Wilson G A, McGranahan N, Birkbak N J, Watkins T B K, Veeriah S, Shafi S, Johnson D H, Mitter R, Rosenthal R, Salm M, Horswell S, Escudero M, Matthews N, Rowan A, Chambers T, Moore D A, Turajlic S, Xu H, Lee S M, Forster M D, Ahmad T, Hiley C T, Abbosh C, Falzon M, Borg E, Marafioti T, Lawrence D, Hayward M, Kolvekar S, Panagiotopoulos N, Janes S M, Thakrar R, Ahmed A, Blackhall F, Summers Y, Shah R, Joseph L, Quinn A M, Crosbie P A, Naidu B, Middleton G, Langman G, Trotter S, Nicolson M, Remmen H, Kerr K, Chetty M, Gomersall L, Fennell D A, Nakas A, Rathinam S, Anand G, Khan S, Russell P, Ezhil V, Ismail B, Irvin-Sellers M, Prakash V, Lester J F, Kornaszewska M, Attanoos R, Adams H, Davies H, Dentro S, Taniere P, O'Sullivan B, Lowe H L, Hartley J A, lles N, Bell H, Ngai Y, Shaw J A, Herrero J, Szallasi Z, Schwarz R F, Stewart A, Quezada S A, Le Quesne J, Van Loo P, Dive C, Hackshaw A, Swanton C, Consortium T R. N Engl J Med. 2017 Jun. 1;376 (22): 2109-21. PMID: 28445112.
18. T-cell invigoration to tumour burden ratio associated with anti-PD-1 response. Huang A C, Postow M A, Orlowski R J, Mick R, Bengsch B, Manne S, Xu W, Harmon S, Giles J R, Wenz B, Adamow M, Kuk D, Panageas K S, Carrera C, Wong P, Quagliarello F, Wubbenhorst B, D'Andrea K, Pauken K E, Herati R S, Staupe R P, Schenkel J M, McGettigan S, Kothari S, George S M, Vonderheide R H, Amaravadi R K, Karakousis G C, Schuchter L M, Xu X, Nathanson K L, Wolchok J D, Gangadhar T C, Wherry E J. Nature. 2017 04/10/online;545:60.
19. High-dimensional single-cell analysis predicts response to anti-PD-1 immunotherapy. Krieg C, Nowicka M, Guglietta S, Schindler S, Hartmann F J, Weber L M, Dummer R, Robinson M D, Levesque M P, Becher B. Nature Medicine. 2018 01/08/online;24:144.
20 Single-Cell Map of Diverse Immune Phenotypes in the Breast Tumor Microenvironment. Azizi E, Carr A J, Plitas G, Cornish A E, Konopacki C, Prabhakaran S, Nainys J, Wu K, Kiseliovas V, Setty M, Choi K, Fromme R M, Dao P, McKenney P T, Wasti R C, Kadaveru K, Mazutis L, Rudensky A Y, Pe'er D. Cell. 2018 Aug. 23; 174 (5): 1293-308 e36. PMID: 29961579.
21. T cell receptor sequencing of early-stage breast cancer tumors identifies altered clonal structure of the T cell repertoire. Beausang J F, Wheeler A J, Chan N H, Hanft V R, Dirbas F M, Jeffrey S S, Quake S R. Proceedings of the National Academy of Sciences. 2017;114 (48): E10409-E17.
22. Landscape of Infiltrating T Cells in Liver Cancer Revealed by Single-Cell Sequencing. Zheng C, Zheng L, Yoo J-K, Guo H, Zhang Y, Guo X, Kang B, Hu R, Huang J Y, Zhang Q, Liu Z, Dong M, Hu X, Ouyang W, Peng J, Zhang Z. Cell. 2017;169 (7): 1342-56.e16.
23. Isolation and characterization of DNA from the plasma of cancer patients. Stroun M, Anker P, Lyautey J, Lederrey C, Maurice P A. European journal of cancer & clinical oncology. 1987 June;23 (6): 707-12. PMID: 3653190.
24. Liquid biopsies come of age: towards implementation of circulating tumour DNA. Wan J C M, Massie C, Garcia-Corbacho J, Mouliere F, Brenton J D, Caldas C, Pacey S, Baird R, Rosenfeld N. Nat Rev Cancer. 2017 April; 17 (4): 223-38. PMID: 28233803.
25. Predicting Radiotherapy Responses and Treatment Outcomes Through Analysis of Circulating Tumor DNA. Chaudhuri A A, Binkley M S, Osmundson E C, Alizadeh A A, Diehn M. Semin Radiat Oncol. 2015 October;25 (4): 305-12. PMID: 26384278.
26. Cell-free nucleic acids as biomarkers in cancer patients. Schwarzenbach H, Hoon D S, Pantel K. Nat Rev Cancer. 2011 June; 11 (6): 426-37. PMID: 21562580.
27. An ultrasensitive method for quantitating circulating tumor DNA with broad patient coverage. Newman A M, Bratman S V, To J, Wynne J F, Eclov N C, Modlin L A, Liu C L, Neal J W, Wakelee H A, Merritt R E, Shrager J B, Loo B W, Jr., Alizadeh A A, Diehn M. Nat Med. 2014 May;20 (5): 548-54. PMID: 24705333.
28. Integrated digital error suppression for improved detection of circulating tumor DNA. Newman A M, Lovejoy A F, Klass D M, Kurtz D M, Chabon J J, Scherer F, Stehr H, Liu C L, Bratman S V, Say C, Zhou L, Carter J N, West R B, Sledge G W, Shrager J B, Loo B W, Jr., Neal J W, Wakelee H A, Diehn M, Alizadeh A A. Nat Biotechnol. 2016 May;34 (5): 547-55. PMID: 27018799.
29. Detection of circulating tumor DNA in early- and late-stage human malignancies. Bettegowda C, Sausen M, Leary R J, Kinde I, Wang Y, Agrawal N, Bartlett B R, Wang H, Luber B, Alani R M, Antonarakis E S, Azad N S, Bardelli A, Brem H, Cameron J L, Lee C C, Fecher L A, Gallia G L, Gibbs P, Le D, Giuntoli R L, Goggins M, Hogarty M D, Holdhoff M, Hong S M, Jiao Y, Juhl H H, Kim J J, Siravegna G, Laheru D A, Lauricella C, Lim M, Lipson E J, Marie S K, Netto G J, Oliner K S, Olivi A, Olsson L, Riggins G J, Sartore-Bianchi A, Schmidt K, Shih I M, Oba-Shinjo S M, Siena S, Theodorescu D, Tie J, Harkins T T, Veronese S, Wang T L, Weingart J D, Wolfgang C L, Wood L D, Xing D, Hruban R H, Wu J, Allen P J, Schmidt C M, Choti M A, Velculescu V E, Kinzler K W, Vogelstein B, Papadopoulos N, Diaz L A, Jr. Sci Transl Med. 2014 Feb. 19;6 (224): 224ra24. PMID: 24553385.
30. Direct detection of early-stage cancers using circulating tumor DNA. Phallen J, Sausen M, Adleff V, Leal A, Hruban C, White J, Anagnostou V, Fiksel J, Cristiano S, Papp E, Speir S, Reinert T, Orntoft M W, Woodward B D, Murphy D, Parpart-Li S, Riley D, Nesselbush M, Sengamalay N, Georgiadis A, Li Q K, Madsen M R, Mortensen F V, Huiskens J, Punt C, van Grieken N, Fijneman R, Meijer G, Husain H, Scharpf R B, Diaz L A, Jr., Jones S, Angiuoli S, Orntoft T, Nielsen H J, Andersen C L, Velculescu V E. Sci Transl Med. 2017 Aug. 16;9 (403). PMID: 28814544.
31. Immune Checkpoint Blockade in Cancer Therapy. Postow M A, Callahan M K, Wolchok J D. J Clin Oncol. 2015 Jun. 10;33 (17): 1974-82. PMID: 25605845.
32. Nivolumab in patients with advanced hepatocellular carcinoma (CheckMate 040): an open-label, non-comparative, phase 1/2 dose escalation and expansion trial. El-Khoueiry A B, Sangro B, Yau T, Crocenzi T S, Kudo M, Hsu C, Kim T Y, Choo S P, Trojan J, Welling T H R, Meyer T, Kang Y K, Yeo W, Chopra A, Anderson J, Dela Cruz C, Lang L, Neely J, Tang H, Dastani H B, Melero I. Lancet. 2017 Jun. 24;389 (10088): 2492-502. PMID: 28434648.
33. Pembrolizumab versus Chemotherapy for P D-L1-Positive Non-Small-Cell Lung Cancer. Reck M, Rodriguez-Abreu D, Robinson A G, Hui R, Csoszi T, Fulop A, Gottfried M, Peled N, Tafreshi A, Cuffe S, O'Brien M, Rao S, Hotta K, Leiby M A, Lubiniecki G M, Shentu Y, Rangwala R, Brahmer J R, Investigators K-N Engl J Med. 2016 Nov. 10;375 (19): 1823-33. PMID: 27718847.
34 Tumor Mutational Burden and Response Rate to PD-1 Inhibition. Yarchoan M, Hopkins A, Jaffee E M. N Engl J Med. 2017 Dec. 21;377 (25): 2500-1. PMID: 29262275.
35. Mechanism-driven biomarkers to guide immune checkpoint blockade in cancer therapy. Topalian S L, Taube J M, Anders R A, Pardoll D M. Nat Rev Cancer. 2016 May; 16 (5): 275-87. PMID: 27079802.
36. Neoantigens in cancer immunotherapy. Schumacher T N, Schreiber R D. Science. 2015 Apr. 3;348 (6230): 69-74. PMID: 25838375.
37. Genomic correlates of response to CTLA-4 blockade in metastatic melanoma. Van Allen E M, Miao D, Schilling B, Shukla S A, Blank C, Zimmer L, Sucker A, Hillen U, Foppen M H G, Goldinger S M, Utikal J, Hassel J C, Weide B, Kaehler K C, Loquai C, Mohr P, Gutzmer R, Dummer R, Gabriel S, Wu C J, Schadendorf D, Garraway L A. Science. 2015 Oct. 9;350 (6257): 207-11. PMID: 26359337.
38. Immune-related response evaluations during immune-checkpoint inhibitor therapy: establishing a “common language” for the new arena of cancer treatment. Nishino M. J Immunother Cancer. 2016; 4:30. PMID: 27330803.
39. New response evaluation criteria in solid tumours: revised RECIST guideline (version 1.1). Eisenhauer E A, Therasse P, Bogaerts J, Schwartz L H, Sargent D, Ford R, Dancey J, Arbuck S, Gwyther S, Mooney M, Rubinstein L, Shankar L, Dodd L, Kaplan R, Lacombe D, Verweij J. Eur J Cancer. 2009 January;45 (2): 228-47. PMID: 19097774.
40. iRECIST: guidelines for response criteria for use in trials testing immunotherapeutics. Seymour L, Bogaerts J, Perrone A, Ford R, Schwartz L H, Mandrekar S, Lin N U, Litiere S, Dancey J, Chen A, Hodi F S, Therasse P, Hoekstra O S, Shankar L K, Wolchok J D, Ballinger M, Caramella C, de Vries E G, group Rw. Lancet Oncol. 2017 March; 18 (3): e143-e52. PMID: 28271869.
41. Pseudoprogression and Immune-Related Response in Solid Tumors. Chiou V L, Burotto M. J Clin Oncol. 2015 Nov. 1;33 (31): 3541-3. PMID: 26261262.
42. Immunotherapy for melanoma. Weber J. Current Opinion in Oncology. 2011; 23 (2): 163-9. PMID: 00001622-201103000-00007.
43. Cancer Immunotherapy: Past Progress and Future Directions. Atkins M B, Sznol M. Seminars in oncology. 2015 August;42 (4): 518-22. Epub 2015 Sep. 1. PMID: 26320057.
44. Immune checkpoint blockade: a common denominator approach to cancer therapy. Topalian S L, Drake C G, Pardoll D M. Cancer cell. 2015 Apr. 13;27 (4): 450-61. Epub 2015 Apr. 11. PMID: 25858804.
45. The Landscape of Actionable Genomic Alterations in Cell-Free Circulating Tumor DNA from 21,807 Advanced Cancer Patients. Zill O A, Banks K C, Fairclough S R, Mortimer S A, Vowles J V, Mokhtari R, Gandara D R, Mack P C, Odegaard J I, Nagy R J, Baca A M, Eltoukhy H, Chudova D I, Lanman R B, Talasaz A. Clinical Cancer Research. 2018;24 (15): 3528-38.
46. Tumor origin detection with tissue-specific miRNA and DNA methylation markers. Tang W, Wan S, Yang Z, Teschendorff A E, Zou Q. Bioinformatics. 2018 Feb. 1;34 (3): 398-406. PMID: 29028927.
47. Plasma DNA tissue mapping by genome-wide methylation sequencing for noninvasive prenatal, cancer, and transplantation assessments. Sun K, Jiang P, Chan K C, Wong J, Cheng Y K, Liang R H, Chan W K, Ma E S, Chan S L, Cheng S H, Chan R W, Tong Y K, Ng S S, Wong R S, Hui D S, Leung T N, Leung T Y, Lai P B, Chiu R W, Lo Y M. Proc Natl Acad Sci USA. 2015 Oct. 6;112 (40): E5503-12. PMID: 26392541.
48. Identification of methylation haplotype blocks aids in deconvolution of heterogeneous tissue samples and tumor tissue-of-origin mapping from plasma DNA. Guo S, Diep D, Plongthongkum N, Fung H L, Zhang K, Zhang K. Nat Genet. 2017 April;49 (4): 635-42. PMID: 28263317.
49. Revealing the vectors of cellular identity with single-cell genomics. Wagner A, Regev A, Yosef N. Nat Biotechnol. 2016 Nov. 8;34 (11): 1145-60. PMID: 27824854.
50 Robust enumeration of cell subsets from tissue expression profiles. Newman A M, Liu C L, Green M R, Gentles A J, Feng W, Xu Y, Hoang C D, Diehn M, Alizadeh A A. Nat Methods. 2015 May; 12 (5): 453-7. PMID: 25822800.
51. Early Detection of Molecular Residual Disease in Localized Lung Cancer by Circulating Tumor DNA Profiling. Chaudhuri A A, Chabon J J, Lovejoy A F, Newman A M, Stehr H, Azad T D, Khodadoust M S, Esfahani M S, Liu C L, Zhou L, Scherer F, Kurtz D M, Say C, Carter J N, Merriott D J, Dudley J C, Binkley M S, Modlin L, Padda S K, Gensheimer M F, West R B, Shrager J B, Neal J W, Wakelee H A, Loo B W, Jr., Alizadeh A A, Diehn M. Cancer Discov. 2017 December;7 (12): 1394-403. PMID: 28899864.
52. Chaudhuri A A, Nabet B Y, Merriott D J, Jin M, Chen E L, Chabon J J, Newman A M, Stehr H, Say C, Carter J N, Walters S, Becker H C, Das M, Padda S K, Loo B W, Jr., Wakelee H A, Neal J W, Alizadeh A A, Diehn M, editors. Circulating tumor DNA quantitation for early response assessment of immune checkpoint inhibitors for lung cancer. American Radium Society Annual Meeting; 2018 May 6, 2018; Orlando, F L.
53. The International Human Epigenome Consortium: A Blueprint for Scientific Collaboration and Discovery. Stunnenberg H G, International Human Epigenome C, Hirst M. Cell. 2016 Nov. 17; 167 (5): 1145-9. PMID: 27863232.
54. Exploring genome wide bisulfite sequencing for DNA methylation analysis in livestock: a technical assessment. Doherty R, Couldrey C. Frontiers in Genetics. 2014 May 13;5 (126).
55. A DNA methylation map of human cancer at single base-pair resolution. Vidal E, Sayols S, Moran S, Guillaumet-Adkins A, Schroeder M P, Royo R, Orozco M, Gut M, Gut I, Lopez-Bigas N, Heyn H, Esteller M. Oncogene. 2017 06/05/online;36:5648.
56. The GEM mapper: fast, accurate and versatile alignment by filtration. Marco-Sola S, Sammeth M, Guigó R, Ribeca P. Nature Methods. 2012 10/28/online;9:1185.
57. gemBS: high throughput processing for DNA methylation data from bisulfite sequencing. Merkel A, Fernández-Callejo M, Casals E, Marco-Sola S, Schuyler R, Gut I G, Heath S C. Bioinformatics. 2018: bty690-bty.
58. NGSCheckMate: software for validating sample identity in next-generation sequencing studies within and across data types. Lee S, Lee S, Ouellette S, Park W-Y, Lee E A, Park P J. Nucleic Acids Research. 2017;45 (11): e103-e.
59. Differential DNA methylation in purified human blood cells: implications for cell lineage and studies on disease susceptibility. Reinius L E, Acevedo N, Joerink M, Pershagen G, Dahlen S E, Greco D, Soderhall C, Scheynius A, Kere J. PLOS One. 2012; 7 (7): e41361. PMID: 22848472.
60. Genome-wide methylation analyses of primary human leukocyte subsets identifies functionally important cell-type-specific hypomethylated regions. Zilbauer M, Rayner T F, Clark C, Coffey A J, Joyce C J, Palta P, Palotie A, Lyons P A, Smith K G. Blood. 2013 Dec. 12; 122 (25): e52-60. PMID: 24159175.
61. Peripheral blood immune cell methylation profiles are associated with nonhematopoietic cancers. Koestler D C, Marsit C J, Christensen B C, Accomando W, Langevin S M, Houseman E A, Nelson H H, Karagas M R, Wiencke J K, Kelsey K T. Cancer Epidemiol Biomarkers Prev. 2012 August;21 (8): 1293-302. PMID: 22714737.
62. Integrative analysis of methylome and transcriptome in human blood identifies extensive sex- and immune cell-specific differentially methylated regions. Mamrut S, Avidan N, Staun-Ram E, Ginzburg E, Truffault F, Berrih-Aknin S, Miller A. Epigenetics. 2015; 10 (10): 943-57. PMID: 26291385.
63. Leukocyte Counts Based on DNA Methylation at Individual Cytosines. Frobel J, Bozic T, Lenz M, Uciechowski P, Han Y, Herwartz R, Strathmann K, Isfort S, Panse J, Esser A, Birkhofer C, Gerstenmaier U, Kraus T, Rink L, Koschmieder S, Wagner W. Clin Chem. 2018 March;64 (3): 566-75. PMID: 29118064.
64. Differential DNA Methylation in Purified Human Blood Cells: Implications for Cell Lineage and Studies on Disease Susceptibility. Reinius L E, Acevedo N, Joerink M, Pershagen G, Dahlen S-E, Greco D, Soderhall C, Scheynius A, Kere J. PLOS ONE. 2012; 7 (7): e41361.
65. Quantitative reconstruction of leukocyte subsets using DNA methylation. Accomando W P, Wiencke J K, Houseman E A, Nelson H H, Kelsey K T. Genome Biol. 2014 Mar. 5;15 (3): R50. PMID: 24598480.
66. Pan-cancer deconvolution of tumour composition using DNA methylation. Chakravarthy A, Furness A, Joshi K, Ghorani E, Ford K, Ward M J, King E V, Lechner M, Marafioti T, Quezada S A, Thomas G J, Feber A, Fenton T R. Nat Commun. 2018 Aug. 13;9 (1): 3220. PMID: 30104673.
67. Distinct DNA methylomes of newborns and centenarians. Heyn H, Li N, Ferreira H J, Moran S, Pisano D G, Gomez A, Diez J, Sanchez-Mut J V, Setien F, Carmona F J, Puca A A, Sayols S, Pujana M A, Serra-Musach J, Iglesias-Platas I, Formiga F, Fernandez A F, Fraga M F, Heath S C, Valencia A, Gut I G, Wang J, Esteller M. Proceedings of the National Academy of Sciences. 2012; 109 (26): 10522-7.
68. Transcriptional Landscape of Human Tissue Lymphocytes Unveils Uniqueness of Tumor infiltrating T Regulatory Cells. De Simone M, Arrigoni A, Rossetti G, Gruarin P, Ranzani V, Politano C, Bonnal Raoul J P, Provasi E, Sarnicola Maria L, Panzeri I, Moro M, Crosti M, Mazzara S, Vaira V, Bosari S, Palleschi A, Santambrogio L, Bovo G, Zucchini N, Totis M, Gianotti L, Cesana G, Perego Roberto A, Maroni N, Pisani Ceretti A, Opocher E, De Francesco R, Geginat J, Stunnenberg Hendrik G, Abrignani S, Pagani M. Immunity. 2016 2016 Nov. 15;45 (5): 1135-47.
69. The cellular and molecular origin of tumor-associated macrophages. Franklin R A, Liao W, Sarkar A, Kim M V, Bivona M R, Liu K, Pamer E G, Li M O. Science. 2014;344 (6186): 921-5.
70. Transcriptomic Analysis Comparing Tumor-Associated Neutrophils with Granulocytic Myeloid-Derived Suppressor Cells and Normal Neutrophils. Fridlender Z G, Sun J, Mishalian I, Singhal S, Cheng G, Kapoor V, Horng W, Fridlender G, Bayuh R, Worthen G S, Albelda S M. PLOS ONE. 2012; 7 (2): e31524.
71. Tumor-associated B-cells induce tumor heterogeneity and therapy resistance. Somasundaram R, Zhang G, Fukunaga-Kalabis M, Perego M, Krepler C, Xu X, Wagner C, Hristova D, Zhang J, Tian T, Wei Z, Liu Q, Garg K, Griss J, Hards R, Maurer M, Hafner C, Mayerhofer M, Karanikas G, Jalili A, Bauer-Pohl V, Weihsengruber F, Rappersberger K, Koller J, Lang R, Hudgens C, Chen G, Tetzlaff M, Wu L, Frederick D T, Scolyer R A, Long G V, Damle M, Ellingsworth C, Grinman L, Choi H, Gavin B J, Dunagin M, Raj A, Scholler N, Gross L, Beqiri M, Bennett K, Watson I, Schaider H, Davies M A, Wargo J, Czerniecki B J, Schuchter L, Herlyn D, Flaherty K, Herlyn M, Wagner S N. Nature Communications. 2017 2017 Sep. 19;8 (1): 607.
72. Circulating tumour DNA methylation markers for diagnosis and prognosis of hepatocellular carcinoma. Xu R H, Wei W, Krawczyk M, Wang W, Luo H, Flagg K, Yi S, Shi W, Quan Q, Li K, Zheng L, Zhang H, Caughey B A, Zhao Q, Hou J, Zhang R, Xu Y, Cai H, Li G, Hou R, Zhong Z, Lin D, Fu X, Zhu J, Duan Y, Yu M, Ying B, Zhang W, Wang J, Zhang E, Zhang C, Li O, Guo R, Carter H, Zhu J K, Hao X, Zhang K. Nat Mater. 2017 November; 16 (11): 1155-61. PMID: 29035356.
73. The Immune Landscape of Cancer. Thorsson V, Gibbs D L, Brown S D, Wolf D, Bortone D S, Ou Yang T H, Porta-Pardo E, Gao G F, Plaisier C L, Eddy J A, Ziv E, Culhane A C, Paull E O, Sivakumar I K A, Gentles A J, Malhotra R, Farshidfar F, Colaprico A, Parker J S, Mose L E, Vo N S, Liu J, Liu Y, Rader J, Dhankani V, Reynolds S M, Bowlby R, Califano A, Cherniack A D, Anastassiou D, Bedognetti D, Rao A, Chen K, Krasnitz A, Hu H, Malta T M, Noushmehr H, Pedamallu C S, Bullman S, Ojesina Al, Lamb A, Zhou W, Shen H, Choueiri T K, Weinstein J N, Guinney J, Saltz J, Holt R A, Rabkin C E, Cancer Genome Atlas Research N, Lazar A J, Serody J S, Demicco E G, Disis M L, Vincent B G, Shmulevich L. Immunity. 2018 Apr. 17;48 (4): 812-30 e14. PMID: 29628290.
74. Comparative analysis of 12 different kits for bisulfite conversion of circulating cell-free DNA. Worm Ørntoft M-B, Jensen SØ, Hansen T B, Bramsen J B, Andersen C L. Epigenetics. 2017 2017 Aug. 3; 12 (8): 626-36.
75. DNABarcodes: an R package for the systematic construction of DNA sample tags. Buschmann T. Bioinformatics. 2017;33 (6): 920-2.

### Example 5: Developing a Liquid Biopsy Approach for Diagnosing and Monitoring Sepsis

### Problem

Sepsis is the most common cause of death in United States hospitals and the number one cause of death worldwide, with 11.0 million sepsis-related deaths reported in 2017. Sepsis is difficult to diagnose and monitor in its early stages, because it is challenging to determine if a patient has an infection (microbial cultures take time to grow), where the infection site is (requires imaging and microbial cultures), and the sites and extent of end-organ damage (often determined clinically, i.e. altered mental status as a marker of brain damage). Unfortunately, when not detected early, patients miss critical early intervention and sepsis progresses rapidly to cause life-threatening multi-organ failure, septic shock, and immunosuppression leading to deadly secondary infections. There are no reliable biomarkers in clinical use for the early diagnosis and monitoring of sepsis.

### Solution

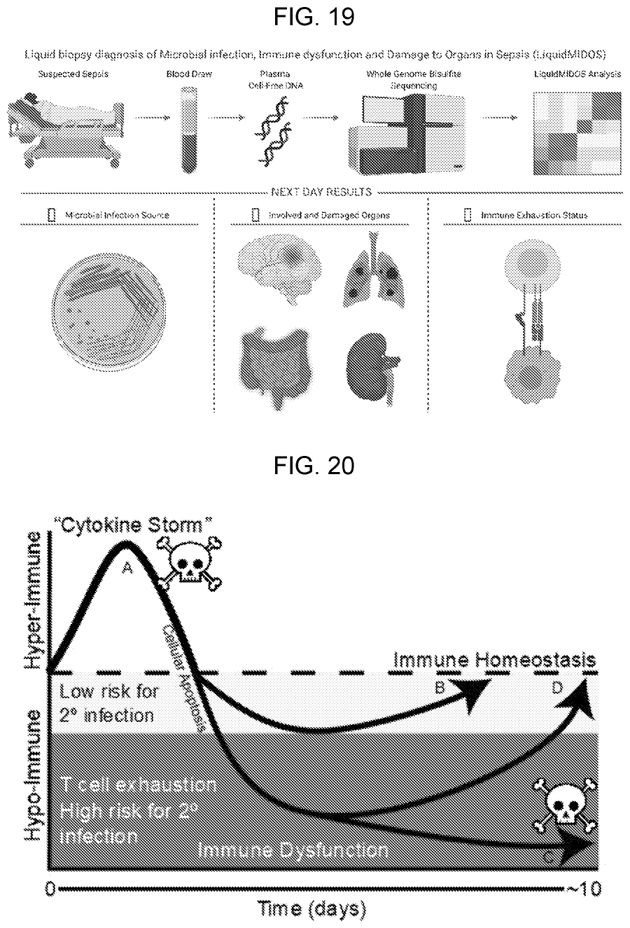

Here, we disclose the development and testing of a liquid biopsy approach called Liquid biopsy diagnosis of Microbial infection, Immune dysfunction, and Damage to Organs in Sepsis (LiquidMIDOS), which will enable the following via whole genome bisulfite sequencing of plasma cell-free DNA (FIG. 19): 1) Detection of the microbial etiology of sepsis; 2) Identification of the septic tissue site; 3) Determination of which organs are getting damaged and thus at risk for failure if not proactively managed; 4) Determination of whether the adaptive immune response against sepsis has become dysfunctional and exhausted, which could in the future be managed precisely with immunotherapy; 5) Detection of deadly secondary infection at its inception. We will compare our method to clinical and laboratory testing performed in the hospital per the standard-of-care. If successful, our approach will enable the early diagnosis and monitoring of sepsis and associated end-organ damage, immune dysfunction, and secondary infection, which should have a direct clinical impact by potentially saving thousands of lives in the United States and millions worldwide.

Sepsis is the most common cause of hospital death in the United States and accounts for 1 in 5 of all deaths worldwide⁴. It is defined as life-threatening organ dysfunction caused by a dysregulated immune response to infection. There were 11 million sepsis-related deaths reported in 2017⁴. Sepsis-associated mortality rates are unacceptably high at 15-25%, and significantly higher for patients diagnosed with associated multi-organ failure⁶⁻⁸. Unfortunately the problem has grown more dire in the year 2020 with ICUs witnessing record numbers of sepsis cases and associated deaths^{9,10}. The most important prognostic factor in sepsis is early intervention, which is impeded by diagnostic challenges. Early diagnosis and intervention are critical to maximize survival in this high-risk patient population.

Diagnosing sepsis depends on a confirmed diagnosis of microbial infection. Infection is typically determined by bacterial cultures which take time to grow: usually 24-72 hours, with some organisms taking 5 days or longer to grow in culture. Bacterial cultures also do not account for other sources of sepsis such as viral infection which have accounted for an increased proportion of septic patients recently¹⁰. Biomarkers suggestive of systemic inflammation such as C-reactive protein, white blood cell count, and procalcitonin have also been tested but have limited sensitivity and specificity, especially at early timepoints and in immunosuppressed settings¹¹⁻¹⁴. It is critical to confirm infection diagnosis early to prevent treatment delays and improve patient survival.

The source of infection can also be difficult to determine early during sepsis, and can require an extensive workup involving chest X-rays, stool cultures, urine cultures, wound cultures, and blood cultures, leading to further diagnostic delays and confusion. Finding the site of infection is an important determinant of management and outcomes, with unknown and pulmonary sites of infection having the highest mortality rates^{15,16}. With LiquidMIDOS, we will thus prioritize determining the infection site and source early.

Even when a clinician suspects sepsis and starts treatment quickly, there is no reliable biomarker to track treatment response. Precisely monitoring sepsis response to treatment is critical for patient survival.

Another important diagnostic factor in sepsis is organ damage. Dysfunction of a single organ can unfortunately progress to multiple organ dysfunction syndrome (MODS) in a septic patient who does not receive adequate upfront care in the acute setting. When this occurs, homeostasis can no longer be maintained, and the patient's prognosis becomes dire. The greater the number of organ systems failing, the higher the mortality rate, with mortality reaching ˜100% when >5 organ systems fail⁷. It is critical to identify organ damage early to prevent MODS and its associated high mortality rate.

Sepsis cases not diagnosed early also had a significantly higher economic burden. In patients diagnosed early (at the time of hospital admission), the cost was $18,023 per patient, but jumped to a staggering $51,022 when the diagnosis was delayed¹⁷. Overall the inpatient cost of sepsis management in U.S. hospitals ranks highest among all disease states, accounting for $24 billion and representing 13% of total U.S. hospital costs in 2013¹⁷. These numbers are likely to balloon further due to the Covid-19 pandemic¹⁰. A major reason is the length of stay and intensive care required for these patients. Diagnosing and monitoring sepsis precisely with an all-in-one assay should help reduce its economic burden in addition to improving patient outcomes.

Sepsis is also an immunological conundrum, with the initial acute phase typically being hyper-immune with a dysregulated immune “cytokine storm” that requires intensive care and causes death from septic shock or multi-organ failure^{5,18,19}. If the patient recovers from this, then this hyper-immune phase is followed days later by a hypo-immune phase characterized by exhausted and dysfunctional T cells, critical cells in the adaptive immune system, which puts patients at risk for deadly secondary infections (FIG. 20)^{5,18-21}. The majority of these dysfunctional/exhausted T cells reside within tissue²⁰, thus a method to detect them sensitively and proactively needs to be capable of querying their tissue sources.

Interestingly, more patients survive the initial acute hyper-immune phase than the subsequent immune-exhausted phase of sepsis⁵. Between 13 and 30% of sepsis patients develop deadly secondary infections, usually from opportunistic microbes that are unlikely to affect someone with a functioning adaptive immune system^{5,22,23}. Flow cytometric and gene expression analyses of peripheral blood cells revealed no differences at early time points^{23,24}, thus querying tissue sources of exhausted immune cells is necessary²⁰; however, biopsies can be dangerous, impractical, and are rarely performed in the acute care setting. It is critical to noninvasively and precisely identify the T cell dysfunctional/exhaustion phase of sepsis to reduce the risk of deadly secondary infection.

Here we will tackle these major challenges with a noninvasive plasma cell-free DNA liquid biopsy approach called LiquidMIDOS. Specifically, LiquidMIDOS will aid in the early diagnosis and monitoring of sepsis by: 1) Detecting the microbial etiology of sepsis; 2) Identifying the septic tissue site; 3) Determining which organs are being damaged; 4) Determining if the T cell response has become dysfunctional; 5) Detecting secondary infection (FIG. 19). LiquidMIDOS will achieve these goals through a single assay from a single blood draw that can be performed early and serially to improve patient survival.

Our approach for developing LiquidMIDOS will take advantage of the fact that tissues from throughout the body continually shed DNA into the circulation, where it can be isolated as cell-free DNA (cfDNA)^{1,25,26} Cell-free DNA is shed into the bloodstream due to cellular turnover and death²⁷. Modern next-generation sequencing (NGS)-based techniques have thus been developed which enable detection of tissue-specific cfDNA at levels as low as ˜0.01% of total cell-free DNA, extracted from a single tube of blood²⁸. Just as tissue cells secrete cfDNA, microbes including bacteria, DNA viruses, fungi, and eukaryotic parasites have also been shown to secrete cfDNA that can be measured through NGS²⁹. We furthermore hypothesized that dysfunctional/exhausted T cells shed cell-free DNA that can be precisely measured by NGS through advanced analytical methods, and distinguished from the much more prevalent cfDNA arising from peripheral blood leukocytes (FIG. 21). Here we describe the quantification of cell-free DNA arising from microbes, organ-specific tissues, and exhausted T cells to proactively determine infection status and etiology, sites of organ involvement and damage, and immunosuppression.

Our method will rely on both cell-free DNA genomics and epigenomics. The epigenome is comprised of chemical compounds bound to the DNA molecule that direct which parts of the genome are turned on vs. off³⁰). Each cell and tissue type has its own unique epigenomic signature³⁰ which can be profiled by analyzing the methylation patterns on DNA using a method called bisulfite sequencing^{31,32}. We can use these epigenomic signatures to detect cfDNA shed by involved/damaged tissue types and exhausted T cells through machine learning-based deconvolution.

Recent published data shows the ability to sensitively detect cancer tissue-of-origin (from among the plethora of different human tissue types) using methylation-based plasma cell-free DNA analysis^{1,28,33}. Additionally, we will achieve the broad dynamic range necessary to measure different levels of organ injury, as shown recently for liver damage (FIG. 22)¹. Recent literature has also shown that genome-wide cell-free DNA sequencing approaches can achieve superb detection sensitivity, comparable to targeted ultra-deep sequencing, because while the sequencing depth is inferior using a genome-wide approach, sequencing the whole genome enables the ability to track a far greater number of specific reporters³⁴. Furthermore, whole genome sequencing of plasma cell-free DNA can be used to sensitively detect multiple infectious microbial species, as shown previously²⁹. Thus performing genome-wide sequencing of cell-free DNA to sensitively detect involved/damaged tissue and microbial sources of sepsis is also supported by recently published literature. Still, no one has shown the ability to detect immune exhaustion by cell-free DNA analysis. Furthermore, LiquidMIDOS will be the first all-in-one method to integrate microbial, immune exhaustion and organ tissue analyses all from a single blood tube with a single assay.

### LiquidMIDOS in the Context of Sepsis

The principles here, however, should be applicable to a plethora of different disease etiologies. We have chosen to focus on sepsis as it is the predominant cause of hospital death in the United States and the most common cause of death worldwide⁴. Focusing on sepsis can allow us to test LiquidMIDOS in the setting where it is poised to make the greatest impact, and for which we have plasma samples with paired clinical data available.

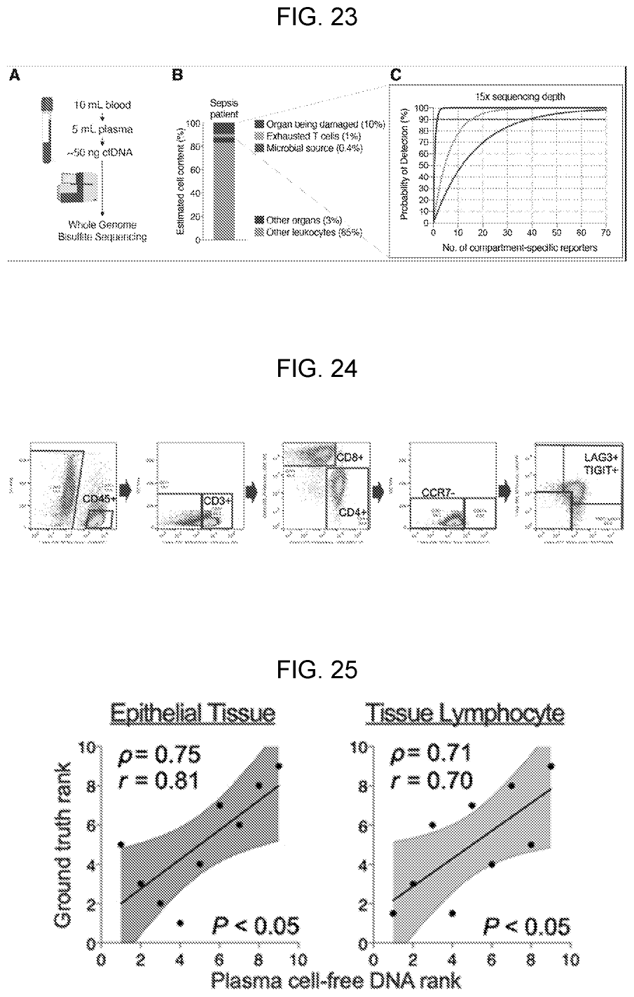

To explore our all-in-one liquid biopsy approach, we first performed a mathematical modeling exercise (FIG. 23). Factors underlying the detection limit of cell-free DNA analysis include the number of independent “reporters” that are interrogated^{34,35}. Using a validated binomial model that was previously described for predicting circulating tumor DNA detection limits³⁵, we estimated the probability of cell-free DNA detection based on the number of unique compartment-specific reporters (i.e., organ tissue-specific differentially methylated regions, microbe-specific genomic sequences), considering: (1) a realistic cell-free DNA input amount (˜50 ng cell-free DNA in 1 blood collection tube³⁵), and (2) 10%¹, 1% and 0.4%² of involved/damaged organ-specific cfDNA, exhausted/dysfunctional T cell-specific cfDNA and microbe-specific cfDNA, respectively. Our mathematical modeling suggests that ≥2 reporters per organ tissue type, ≥15 specific to exhausted T cells, and ≥40 microbe-specific genomic motifs will enable sensitive and specific detection with ≥90% probability. Our model suggests that LiquidMIDOS will enable sensitive cell-free DNA detection, especially given the millions of potential compartment-specific reporters available genome-wide³⁴.

We then asked if we could achieve high-quality sequencing results using the banked plasma samples available to us. We first showed we could reliably achieve 4050 sequencing depth when targeting this by multiplex sequencing on an Illumina NovaSeq S4 flow cell, with DNA inputs into library preparation ranging between 30 ng and 120 ng using the Accel-NGS Methyl-Seq workflow (Swift Biosciences). We then asked another practical question-Does freezing affect the ability to reliably measure methylation patterns? To answer this, we performed whole genome bisulfite sequencing (WGBS) of 9 peripheral blood leukocyte samples from a healthy donor, with all sample preparation performed fresh (without freezing) on 3 samples, DNA frozen for 3 samples, and the cells cryopreserved prior to further processing for the remaining 3 samples. Following sequencing analysis, we observed no major differences in global methylation patterns (FIG. 15), suggesting that cryo-banking cells or DNA does not introduce epigenomic artifacts, consistent with prior literature³⁶.

We next asked if distinct methylation reporters could be identified in tissue-derived epithelial cells, tissue lymphocytes enriched for exhausted T cells, and normal peripheral blood leukocytes (PBLs). This was important to establish that epigenomic signatures were distinct between these three classes of cells. We thus performed flow cytometry and isolated epithelial cells, PBLs and tissue lymphocytes from 10 patients with oligometastatic colorectal cancer. To focus on exhausted T cells, we developed a flow cytometric approach to specifically sort these cells from tissue prior to sequencing (FIG. 24). We then performed WGBS on each sample, followed by differential methylated region (DMR) analysis, and identified the 70 most differentially methylated CpG positions (FIG. 1). This revealed that epithelial cells, tissue lymphocytes (enriched for dysfunctional/exhausted T cells) and PBLs have distinct methylation profiles, suggesting we can use WGBS to distinguish between them epigenomically.

We next queried whether signal from epithelial tissue and tissue lymphocytes enriched for exhausted T cells can be detected in cell-free DNA. To do this, we isolated plasma cell-free DNA from 13 patients with oligometastatic colorectal cancer, and performed WGBS on an Illumina NovaSeq S4 flow cell targeting 4050 genome-wide coverage. We deconvolved this data by querying the specific epithelial tissue vs. tissue lymphocyte vs. PBL reporters shown in FIG. 1 using CIBERSORTx³⁷. Using this approach, we were able to detect leukocyte-derived cfDNA from all patients, epithelial tissue-derived cfDNA from 9 of 13 patients, and tissue lymphocyte-derived cfDNA from 9 of 13 patients (FIG. 2A). Moreover, the levels of epithelial-derived and tissue lymphocyte-derived cfDNA using our methylated cell-free DNA deconvolution approach correlated significantly with ground truth determined by tumor flow cytometry and sum of longest tumor diameters (FIG. 25). Indicative of methodological specificity, the same analysis performed on 12 healthy donor plasma cfDNA samples showed only PBL-specific signal with no evidence of epithelial tissue-derived cfDNA or tissue lymphocyte-specific cfDNA (FIG. 2A). Our data indicate that LiquidMIDOS has the potential to detect both tissue-derived and exhausted tissue lymphocyte-derived cell-free DNA, and accurately discriminate these from the more dominant PBL signal in blood plasma.

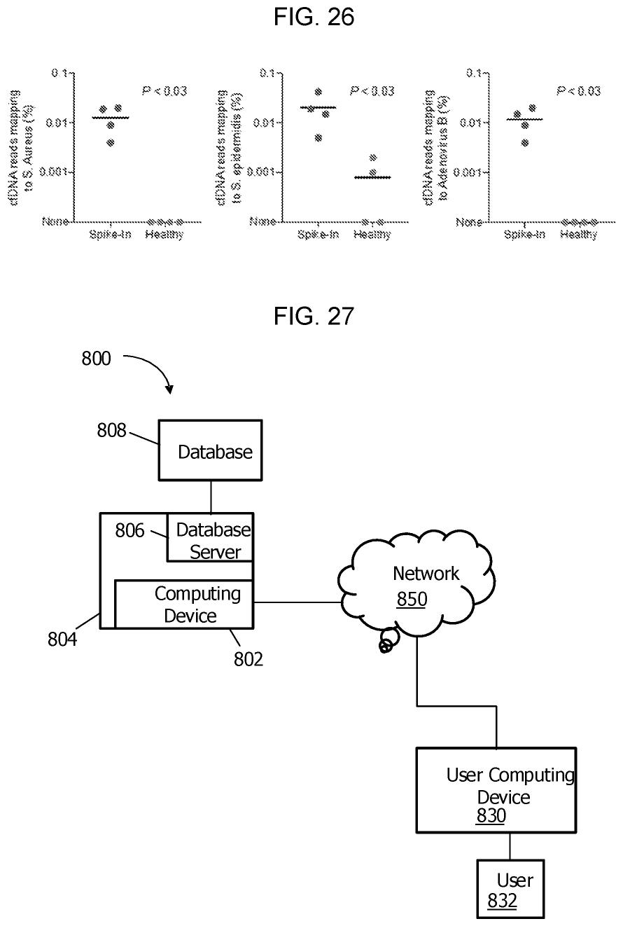

We next queried whether we could detect microbial DNA within plasma cell-free DNA as part of our sequencing workflow. To do this, we focused on Staphylococcus aureus, among the most common virulent types of bacteria causing sepsis. We also focused on Staphylococcus epidermidis, an avirulent pathogen that normally colonizes human skin, but can become pathogenic during the immunosuppressive phase of sepsis. Another pathogen we focused on was Adenovirus B, which usually causes the common cold, but can become deadly in the setting of immunosuppression. Focusing our analysis on these three important causes of primary and secondary sepsis, we analyzed publicly available whole genome sequencing of human plasma cell-free DNA with sheared microbial DNA spiked in at low concentrations ranging between 32 and 1,000 molecules per microliter of plasma (https://www.ncbi.nlm.nih.gov/bioproject/PRJNA507824). Samples were sequenced on a NextSeq 500 with 750,000 reads on average per sample. We then aligned the sequencing reads along with cell-free DNA from 4 healthy donors against microbial genomes in the NCBI microbial genome resource using megaBLAST³⁸. As expected, this revealed that all human plasma samples with low levels of sheared microbial DNA had detectable reads that mapped to those organisms with >90% identity (FIG. 26). In contrast, four healthy donors with no spiked-in microbial DNA had no evidence of genomic motifs specific to these microbial organisms, except for significantly lower levels of Staphylococcus Epidermidis (a normal skin-dwelling bacterium) in 2 of the 4 healthy donors, indicative of high methodological specificity (FIG. 26). These data suggest that our genome-wide method for cell-free DNA analysis will sensitively detect DNA from microbes underlying sepsis, including secondary infection arising in the setting of immunosuppression.

We can significantly extend this initial work to develop a blood-based all-in-one sepsis detection and monitoring assay called LiquidMIDOS that gives the clinician data regarding the microbial and tissue sources, sites of end-organ damage, and the extent and timing of T cell dysfunction/exhaustion. LiquidMIDOS will be clinically useful, serving as a clinician's “Swiss Army knife” for data-driven diagnosis, monitoring, and management of sepsis (Table 1).

TABLE 1
How LiquidMIDOS results can be used to answer
clinically important questions in sepsis.

| | Answer using LiquidMIDOS | |
|---|---|---|
| Clinical Question | Biomarker (+)/Rising | Biomarker (−)/Falling |
| Does the patient | Yes | No |
| have sepsis? | | |
| Is the patient | cfDMA analysis detects | cfDNA analysis detects |
| infected? | microbial pathogen | no microbial pathogen |
| What is the | cfDNA is elevated from | cfDNA is not elevated |
| infection source? | involved organ tissue | from uninvolved organ |
| Are organs being | cfDMA is elevated from | cfDNA is not elevated |
| damaged? | damaged organ tissue | from undamaged organ |
| Is the patient | No | Yes |
| responding to | | |
| treatment? | | |
| Is the infection | Microbial cfDNA levels | Microbial cfDNA levels |
| improving? | increasing | decreasing |
| Is organ damage | cfDNA from | cfDNA from |
| improving? | involved/damaged | involved/damaged |
| | organs rising | organs failing |
| Is the patient's | Yes | No |
| condition | | |
| worsening? | | |
| Is the patient at risk | cfDNA is high from | cfDNA is not high from |
| for multi-organ | multiple organ systems | multiple organ systems |
| failure? | | |
| Will patient develop | cfDMA is detectable | cfDNA is not detectable |
| secondary infection? | from exhausted T cells | from exhausted T cells |
| | cfDMA is detectable | No cfDNA detectable |
| | from opportunistic | from opportunistic |
| | pathogen | pathogen |

For LiquidMIDOS to function robustly, it will require distinct input signatures derived from our cell types of interest. We will thus begin by analyzing tissue and lymphocyte sources profiled by WGBS in the Encode³⁹, Blueprint⁴⁰, and NIH Roadmap Epigenomics Project³⁰ databases. These represent nearly all normal human tissue and leukocyte cell types. We will additionally use fluorescence-activated cell sorting (FACS) to isolate exhausted T cells from infection-involved tissues that were cryopreserved immediately post-mortem from sepsis patients (using a schema similar to FIG. 24). We will sequence these sorted exhausted T cells by WGBS. Using these data (WGBS from multiple tissue sources, normal peripheral blood leukocytes, and exhausted tissue-resident T cells from sepsis patients), we will apply Metilene⁴¹ for DMR analysis. This will be followed by refinement of cell type-specific methylation reporter profiles using machine learning feature selection approaches, including random forests and elastic net, to yield a signature matrix (similar conceptually to FIG. 1) that we can use to deconvolve patient-derived plasma cell-free DNA WGBS data using CIBERSORTx³⁷. This will identify promoter regions specifically hypo- or hyper-methylated in each cell/tissue type of interest, i.e., PDCD1, CTLA4, TIGIT, LAG3, and TIM3 in exhausted T cells⁴². To confirm the biological relevance of cell/tissue-specific reporters identified by machine learning for our signature matrix, we will perform literature searching as well as gene set enrichment analysis using the ToppGene Suite⁴³. These specific methylation reporters will allow us to distinguish and quantify cell/tissue types relevant to sepsis from cell-free DNA.

To determine the sample size needed to derive a signature matrix that can capably distinguish between different categories of cells/tissue, we had to estimate the effect size, which we did by examining our data profiling tissue lymphocytes vs. epithelial cells vs. PBLs in colon cancer patients (FIG. 1); this suggests a large effect size with clear discrimination between groups per methylation status of the most discriminatory reporter positions. However, to be conservative, and given we will attempt to distinguish between multiple types of organ tissue (not just general epithelial cells) we will assume a medium Cohen's d effect size of 0.544. This yields a result of n=18 per group to achieve 0.90 power at a=0.05. We will analyze WGBS data from n=18 per cell/tissue type to derive the signature matrix for LiquidMIDOS. We expect that greater power will likely be achieved given the large effect size observed in FIG. 1, and the robust ability to discriminate between different human tissue types through methylation-based cell-free DNA analysis in other studies^{1,26,33} that queried far fewer CpG sites (through targeted sequencing or microarrays) than we plan to by WGBS.

Two Banked Cohorts of Blood Samples from Sepsis Patients for Training and Validation of LiquidMIDOS Method

We have collected these samples at Washington University for the past 5 years. Plasma and PBLs were separated from each other, processed, and cryo-stored immediately after collection using a standardized protocol. To date, we have banked samples from ˜100 sepsis patients. Nearly all sepsis patients in our bank have serial blood plasma and peripheral blood leukocytes collected daily in the ICU starting from day 1 of admission, with fully annotated paired clinical and survival data. We also have banked samples from ˜100 propensity-matched non-sepsis controls. We furthermore have access to separate similarly-sized and annotated cohorts from Yale Medical Center that we will utilize for methodological validation. From both cohorts, we have access to banked autopsy samples from a subset of sepsis patients, which we can utilize to confirm microbial etiologies of infection, organs involved and damaged by sepsis, and dysfunctional/exhausted T cell status. Overall, we have the necessary ground-truth data for training and testing LiquidMIDOS (Table 2).

TABLE 2
Clinical parameters and their ground-truth details
in the training and validation data sets.

| Parameter | Ground-Truth | Details on Ground-Truth |
|---|---|---|
| Sepsis status | ✓ | Determined by a board-certified |
| | | ICU physical and confirmed |
| | | retrospectively |
| Microbial | ✓ | Positive pathogenic bacterial/viral |
| etiology | | cultures or PCR reaction |
| Organs infected | ✓ | Confirmed by imaging or |
| | | tissue-specific microbial analysis |
| Organs damaged | ✓ | Determined by a board-certified |
| | | ICU physician, and confirmed |
| | | retrospectively. |
| Dysfunctional | ✓ | Determined retrospectively by |
| immunity | | diagnosis of secondary |
| | | opportunistic infection. |
| Days in the ICU | ✓ | Determined retrospectively |
| | | by medical record review. |
| 30-day mortality | ✓ | Determined retrospectively |
| | | by medical record review. |
| Autopsy samples | ✓ | Available from a subset of |
| | | patients. |
| Daily lab test | ✓ | Available for all patients. |
| results | | |
| Non-sepsis | ✓ | Confirmed retrospectively |
| control status | | by a board-certified MD> |

To train LiquidMIDOS, we will apply it to plasma cell-free DNA samples from ˜100 sepsis patients from Washington University, collected daily from day 1 of ICU admission. We will perform WGBS on each of these samples, and then perform LiquidMIDOS analysis to determine: 1) The microbial etiology of infection (by applying BLAST³⁸ to human-off-target reads against the NCBI microbial database; 2) The organs involved/damaged—by determining which organ tissue sources predominantly contribute to plasma cell-free DNA; 3) Dysfunctional status of the immune system—by quantifying exhausted T cell-derived cell-free DNA. We will correlate our predictions with ground-truth in our clinical cohort (Table 2). We will do this correlative analysis on a per-time-point basis, possible given the high level of clinical and laboratory annotation we have. To train our method's specificity, we will separately apply LiquidMIDOS to blood plasma samples acquired from ˜100 propensity score-matched controls.

Specifically, we will extract cell-free DNA from plasma samples using the QIAamp Circulating Nucleic Acid Kit (Qiagen), and then perform library preparation using the Accel-NGS Methyl-Seq DNA Library Kit (Swift Biosciences). Samples will be barcoded such that they can be sequenced in a multiplexed fashion on a NovaSeq S4 flow cell (Illumina) targeting 4050 depth (˜40 samples per flow cell). We will apply standard NGS quality control (QC) filters, and then map sequencing reads to the human genome. QC-passing human-unmapped reads will then be aligned to the NCBI microbial database (https://www.ncbi.nlm.nih.gov/genome/microbes) using BLAST³⁸; the number of reads that align to a microbial genome, divided by the total number of QC-passed sequencing reads for the sample will be used to quantify the percentage of plasma cfDNA arising from that microbe⁴⁵. Thus we will determine microbial content via plasma cfDNA analysis of our training cohort.

We will next query methylation patterns within the human-mapped sequencing reads that passed QC. Given the case-control nature of our study, it is important to guard against batch effects which could confound our results. We will thus compare sequencing depth and fragment size distributions in our sepsis patients (cases) vs. non-sepsis patients (controls) using samtools mpileup⁴⁶. If these are systematically different, we will apply filtration and normalization techniques before proceeding, for example by removing reads >300 base pairs in size and/or down-sampling mapped reads to the lowest common denominator before further analysis. We will furthermore systematically compare methylation levels in housekeeping genes between case and control samples, and compare their promoter methylation levels and variances. If we observe that batch effects are persisting, we will utilize a bioinformatic batch correction strategy such as COMBAT⁴⁷. This is important to ensure that differences we see in our case-control study design are not the result of batch effects.

We will next deconvolve our QC-passing human-mapped reads from cell-free DNA WGBS using CIBERSORTx³⁷ with our LiquidMIDOS-specific signature matrix. To determine relative abundances of each queried organ tissue type, and dysfunctional/exhausted T cells, we will quantify their relative abundances as outputted by CIBERSORTx³⁷ after normalizing out the predominant PBL-derived signal.

We will then apply machine learning to our case vs. control cell-free DNA results to develop a LiquidMIDOS classifier to predict sepsis from non-sepsis along with associated predictive/prognostic metrics. The observed differences will be sepsis-specific as the cohorts are otherwise propensity score-matched. We will develop an optimized classifier by applying different machine learning techniques including Bayesian classification, generalized linear model, k-means classification, logistic regression, support vector machine, random forest, and principal component analysis, with keen attention to distinctly classifying the clinically important parameters of: sepsis status, microbial infection sources, organ tissue sites of involvement/damage, and immunosuppression status (see Table 2). We will also perform goodness-of-fit testing to assess prognostic accuracy using the Hosmer-Lemeshow test for binary outcomes such as 30-day mortality⁴⁸. Following assessment of methodological accuracy, we will determine which machine learning technique classifies our training data best, and utilize it in our final LiquidMIDOS method. We will compare the resulting LiquidMIDOS score to laboratory tests standardly utilized by clinicians when diagnosing and monitoring sepsis¹⁴ (C-reactive protein level, white blood cell count, procalcitonin level, lactate level) with the primary criterion for comparison being the ability to distinguish sepsis patients from non-sepsis controls. This will be assessed by testing whether the AUC/C-index is statistically significantly greater than 0.5. We will identify LiquidMIDOS's optimal classification cutpoint using Youden's index (and report the associated sensitivity and specificity); we will do this with regard to each of the criteria displayed in Table 1, as well as in a time-dependent manner (using our serial samples) to determine if the LiquidMIDOS classification scores change over time as would be expected in Table 1. Using this training cohort, we will develop a high-performance blood-based all-in-one LiquidMIDOS classifier and monitoring tool for sepsis.

While we expect cell-free DNA to be the optimal blood-based analyte for our LiquidMIDOS assay, it is possible that some aspects might perform better in the PBL compartment. We believe this to be unlikely, as cell-free DNA has been shown to represent human cell/tissue turnover from throughout the body^{1,26,27}, and exhausted T cells are thought to be much more prevalent within tissue than in circulation in sepsis-mediated immunosuppression^{20,23}. Still, if it is the case that some aspects of LiquidMIDOS are more sensitive from the PBL compartment, LiquidMIDOS would still be possible to perform from a single blood draw, as the plasma and PBLs are isolated from the same tube of blood, although some of the workflow would need to be replicated (WGBS performed on plasma- and PBL-derived DNA separately). Still, to ensure our assay is as sensitive as possible, we will sequence, deconvolve and classify PBL-derived sheared DNA using the same workflow described above. We will thus query whether cell-free DNA is a superior analyte to PBLs for LiquidMIDOS, and will flexibly proceed with the most sensitive analyte in a setting-dependent manner.

We will next validate LiquidMIDOS by applying it to a held-out cohort of ˜100 sepsis and ˜100 non-sepsis patients from Yale Medical Center. Similar to the training cohort, sepsis patients underwent daily plasma and PBL collection starting from day 1 of ICU admission. We will perform propensity score matching to ensure that cases and controls are overall matched in terms of clinical and epidemiological covariates other than sepsis-specific factors. We will again perform WGBS on each of these samples, focusing on the sample types (plasma vs. PBLs) that performed best in the above training exercise and apply sequencing deconvolution and LiquidMIDOS-based classification as described above (but using the machine learning-optimized cutpoints from our training cohort) to determine: 1) sepsis status of the patient; 2) microbial sources of infection; 3) organs involved/damaged; 4) suppressed status of the immune system. We will again correlate our predictions on a per-time-point basis with ground-truth (Table 2) including prognosis assessed by 30-day mortality. We will also validate whether increases/decreases in LiquidMIDOS scores correlate with worse/better outcomes across different metrics as would be expected in Table 1. We will similarly test the propensity score-matched non-sepsis patient blood samples to validate our method's specificity, again using the LiquidMIDOS score cutpoints determined in our training cohort. We will compare our method's ability to predict outcomes and classify sepsis vs. non-sepsis, compared to laboratory tests standardly ordered by clinicians when diagnosing and monitoring sepsis¹⁴: C-reactive protein, white blood cell count, procalcitonin, lactate. We can validate LiquidMIDOS in an independent clinical cohort, showcasing our blood-based all-in-one method for sepsis diagnosis and monitoring, while demonstrating superiority over standard-of-care laboratory testing.

As described above, we have access to two well-annotated clinical cohorts (Table 2) and can generate a comprehensive blood-based microbial and human sequencing repository for sepsis with paired clinical correlative data, and propensity-matched controls. Such a data set doesn't currently exist and will serve as an invaluable resource to the scientific community for this and other innovative work.

### Cost-Effective Approach

We estimate the cost of LiquidMIDOS to be $2,000 per assay based on estimates of library preparation, sequencing, and genomic analysis. As mentioned above, sepsis cases not diagnosed early had a significantly higher economic burden, costing $51,022 per patient, compared to $18,023 when sepsis was accurately diagnosed at the time of hospital admission¹⁷. Delayed diagnoses are associated with increased sepsis severity, longer hospital and ICU stays, and inferior survival. If we conservatively assume that among patients with late-diagnosed sepsis (costing $51,022 per patient), LiquidMIDOS serial monitoring ×3 reduces the cost in 25% to the baseline level of $18,023 per patient (+$6,000 of assay costs), then utilizing LiquidMIDOS would save on average $2,250 per patient. We expect the actual cost-savings with LiquidMIDOS to be even greater in the clinical setting as the assay becomes more streamlined for CLIA-certified and CAP-accredited laboratory workflows and NGS costs continue to plummet⁴⁹, thus reducing the significant cost burden of sepsis on the American health system.

Our approach can also be used in the clinical setting given the increased prevalence of genomics-based assays in acute care settings, including the commercial Karius assay for microbial detection from cell-free DNA²⁹. With the increased sophistication of molecular pathology laboratories in hospitals, many of which have their own next-generation sequencers, we expect that the turn-around-time for our assay will be 24 hours (same as the next-day turn-around-time of the whole genome sequencing-based microbial cfDNA assay offered by Karius²⁹). While this will initially be too slow to ensure point-of-care diagnosis, it should act as a rapid confirmatory test of diagnosis, and an efficient all-in-one sepsis monitoring tool. As NGS speed increases due to improved technology, and LiquidMIDOS is implemented within highly streamlined CLIA-certified and CAP-accredited laboratory workflows, we expect turn-around-time to be even faster, with results potentially available within hours, similar to most other laboratory tests ordered in the hospital in the acute care setting.

### REFERENCES

1. Moss, J., et al., 2018.
2. Grumaz, S., et al., 2016.
3. Crowley, E., et al., 2013.
4. Rudd, K. E., et al., 2020.
5. Boomer, J. S., et al., 2014.
6. Hotchkiss, R. S., et al., 2016.
7. Bilevicius, E., et al., 2001.
8. Caraballo, C., et al., 2019.
9. Alhazzani, W., et al., 2020.
10. Bhatraju, P. K., et al., 2020.
11. Blommendahl, J., et al., 2002.
12. Lai, L., et al., 2020.
13. Svaldi, M., et al., 2001.
14. Pierrakos, C., et al., 2010.
15. Caraballo, C., et al., 2019.
16. Mayr, F. B., et al., 2014.
17. Paoli, C. J., et al., 2018.
18. Cao, C., et al., 2019.
19. Delano, M. J., et al., 2016.
20. Boomer, J. S., et al., 2011.
21. Denstaedt, S. J., et al., 2018.
22. Chen, Y., et al., 2019.
23. van Vught, L. A., et al., 2016.
24. Boomer, J. S., et al., 2012.
25. Swarup, V., et al., 2007.
26. Sun, K., et al., 2015.
27. Rostami, A., et al., 2020.
28. Shen, S. Y., et al., 2018.
29. Blauwkamp, T. A., et al., 2019.
30. Bernstein, B. E., et al., 2010.
31. Deng, J., et al., 2009.
32. Gu, H., et al., 2011.
33. Liu, M. C., et al., 2020.
34. Zviran, A., et al., 2020.
35. Newman, A. M., et al., 2016.
36. Li, Y., et al., 2018.
37. Newman, A. M., et al., 2019.
38. McGinnis, S., et al., 2004.
39. Consortium, E. P., 2004.
40. Martens, J. H., et al., 2013.
41. Juhling, F., et al., 2016.
42. Wherry, E. J., et al., 2015.
43. Chen, J., et al., 2009.
44. Cohen, J., 1988.
45. Johnson, M., et al., 2008.
46. Li, H., et al., 2009.
47. Johnson, W. E., et al., 2007.
48. D'Agostino, R. B., et al., 2001.
49. van Nimwegen, K. J., et al., 2016.
50. Chaudhuri, A. A., et al., 2017.
51. Dang, H. X., et al., 2020.
52. Barnell, E. K., et al., 2019.

### Example 6: Cell-Free DNA Epigenomics to Track the Dynamics of Organ Damage and Immune Exhaustion During Sepsis

Sepsis is the most common cause of hospital death in the United States and accounts for 1 in 5 of all deaths worldwide². It is an immunological conundrum, with the initial acute phase typically being hyper-immune with a dysregulated immune “cytokine storm” that requires intensive care and can lead to death from septic shock or multi-organ failure³⁻⁵. If the patient recovers from this, then this hyper-immune phase is followed days later by a hypo-immune phase characterized by exhausted and dysfunctional T cells, critical cells in the adaptive immune system, which puts patients at risk for deadly secondary infections³⁻⁷ (FIG. 20). The majority of these dysfunctional and exhausted T cells reside within organ tissues.

Interestingly, more patients survive the initial acute hyper-immune phase than the subsequent immune-exhausted phase of sepsis³. Between 13 and 30% of sepsis patients develop deadly secondary infections, usually from opportunistic microbes that are unlikely to affect someone with a functioning adaptive immune system^{3,9,10}. Flow cytometric and gene expression analyses of peripheral blood cells revealed no differences at early time points^{10,11}, thus querying tissue sources of exhausted immune cells is necessary⁶; however, biopsies can be dangerous, impractical, and are rarely performed in the acute care setting. It is critical to noninvasively and precisely identify the T cell dysfunctional/exhaustion phase of sepsis to reduce the risk of deadly secondary infection.

Our approach will take advantage of the fact that tissues from throughout the body continually shed DNA into the circulation, where it can be isolated as cell-free DNA (cfDNA)^{1,16,17}. Cell-free DNA is shed into the bloodstream due to cellular turnover and death¹⁸. Modern next-generation sequencing (NGS)-based techniques have thus been developed which enable detection of tissue-specific cfDNA at levels as low as ˜0.01% of total cell-free DNA, extracted from a single tube of blood¹⁹. Just as tissue cells shed cfDNA, infectious microbes have also been shown to shed cfDNA that can be measured through NGS²⁰. We furthermore hypothesized that dysfunctional/exhausted T cells shed cell-free DNA that can be precisely measured by NGS through advanced analytical methods, and distinguished from the much more prevalent cfDNA arising from peripheral blood leukocytes (FIG. 21). Here, we describe the quantification of cell-free DNA arising from organ-specific tissues and exhausted T cells in order to noninvasively track organ damage and immune dysfunction/exhaustion, respectively, during sepsis.

Our methods will rely on cell-free DNA epigenomics. The epigenome is comprised of chemical compounds bound to the DNA molecule that direct which parts of the genome are turned on vs. off²¹. Each cell and tissue type has its own unique epigenomic signature²¹ which can be profiled by analyzing the methylation patterns on DNA using a method called whole genome bisulfite sequencing (WGBS)^{22,23}. We can use these epigenomic signatures to detect cell-free DNA shed by involved/damaged tissue types and dysfunctional/exhausted T cells through machine learning-based deconvolution.

Recently published data shows the ability to sensitively detect cancer tissue-of-origin (from among the plethora of different human tissue types) using methylation-based cell-free DNA analysis^{1,19,24}. Additionally, we should achieve the broad dynamic range necessary to measure different levels of organ injury, as shown recently for liver damage using a more elementary methylation microarray approach applied to cfDNA¹ (FIG. 22). Recent literature has also shown that genome-wide cell-free DNA sequencing approaches can achieve 90% detection sensitivity for minimal residual disease²⁵, comparable to targeted ultra-deep sequencing^{12,13,26}, because while the sequencing depth is inferior using a genome-wide approach, sequencing the whole genome enables the ability to track a far greater number of specific reporters²⁵. Furthermore, whole genome sequencing of cell-free DNA can be used to sensitively detect infectious microbial species in sepsis²⁰. Thus performing genome-wide sequencing of cell-free DNA to sensitively detect involved/damaged tissue can be achieved.

### Data

Specifically, we asked if methylation reporters could distinguish exhausted tissue lymphocytes from tissue-derived epithelial cells and normal peripheral blood leukocytes (PBLs). We thus performed flow cytometry and isolated epithelial cells, PBLs, and tissue lymphocytes from 10 patients with oligometastatic colorectal cancer. To focus on exhausted T cells, we developed a flow cytometric approach to specifically sort these cells from tissue prior to sequencing (FIG. 24). We then performed WGBS on each sample, followed by differential methylated region (DMR) analysis, and identified the 70 most differentially methylated CpG positions (FIG. 1). This revealed that epithelial cells, tissue lymphocytes (enriched for dysfunctional/exhausted T cells) and PBLs have distinct methylation profiles, suggesting we can use WGBS to distinguish between them.

We next queried whether the epigenomic signals from epithelial tissue and from tissue lymphocytes enriched for exhausted T cells can be detected in cell-free DNA. To do this, we isolated plasma cell-free DNA from 13 patients with oligometastatic colorectal cancer and performed WGBS on an Illumina NovaSeq S4 flow cell targeting 4050 genome-wide coverage. We deconvolved this data by querying the specific epithelial tissue vs. tissue lymphocyte vs. PBL reporters shown in FIG. 1 using CIBERSORTx²⁷. Using this approach, we were able to detect PBL-derived cfDNA from all patients, epithelial tissue-derived cfDNA from 9 of 13 patients, and tissue lymphocyte-derived cfDNA from 9 of 13 patients (FIG. 2A). Moreover, the levels of epithelial-derived and tissue lymphocyte-derived cfDNA using our methylated cell-free DNA deconvolution approach correlated significantly with ground truth determined by tumor flow cytometry and sum of longest tumor diameters (FIG. 25). Indicative of methodological specificity, the same analysis performed on 12 healthy donor cfDNA samples showed only PBL-specific signal with no evidence of epithelial tissue-derived cfDNA or tissue lymphocyte-specific cfDNA (FIG. 2A). Our data indicate that we can use WGBS to detect both tissue-derived and exhausted tissue lymphocyte-derived cell-free DNA, and accurately discriminate these from the more dominant PBL signal in blood plasma.

This work can be significantly extended to query the kinetics/dynamics of end-organ damage, and separately T cell dysfunction/exhaustion during sepsis.

For our cell-free DNA based genome-wide methylation deconvolution approach to function robustly, it will require distinct input signatures derived from our cell types of interest which we will input into CIBERSORTx²⁷. We will thus begin by analyzing tissue and lymphocyte sources profiled by WGBS in the Encode³⁰, Blueprint³¹ and NIH Roadmap Epigenomics Project²¹ databases. These represent nearly all human tissue and leukocyte cell types. Using these data (WGBS from multiple tissue sources, normal peripheral blood leukocytes, and exhausted tissue-resident T cells), we will apply Metilene³² for differential methylated region analysis. This will be followed by refinement of cell type-specific methylation reporter profiles using machine learning feature selection approaches, including random forests and elastic net, to yield a signature matrix (similar conceptually to FIG. 1) that we can use to deconvolve patient-derived plasma cell-free DNA WGBS data using CIBERSORTx²⁷. This will identify promoter regions specifically hypo- or hyper-methylated in each cell/tissue type of interest, i.e. PDCD1, CTLA4, TIGIT, LAG3 and TIM3 in exhausted T cells³³. These specific methylation reporters will allow us to distinguish and quantify cell/tissue types relevant to sepsis from cell-free DNA.

To determine the sample size needed to derive a signature matrix that can capably distinguish between different categories of cells/tissues, we had to estimate the effect size, which we did by examining our data profiling tissue lymphocytes vs. epithelial cells vs. PBLs in colon cancer patients (FIG. 1); this suggests a large effect size with clear discrimination between groups per methylation status of the most discriminatory reporter positions. However, to be conservative and given that cancer is a fundamentally different etiology than sepsis, and because we will attempt to distinguish between multiple types of organ tissue (not just general epithelial cells) we will assume a medium Cohen's d effect size of 0.534. This yields a result of n=18 per group to achieve 0.90 power at a=0.05. We will thus plan to analyze WGBS data from n=18 per cell/tissue type to derive the signature matrix. We expect that greater power will likely be achieved given the large effect size observed in FIG. 1, and the robust ability to discriminate between human tissue types via methylation-based cell-free DNA analysis in other studies^{1,19,24} that queried fewer CpG sites (through targeted sequencing or microarrays) than we will by WGBS.

We will next utilize a banked cohort of blood samples from sepsis patients, with paired clinical data (Table 2, see Example 5). We have been collecting these samples at Washington University for the past 5 years. Plasma and PBLs were separated from each other, processed, and cryo-stored immediately after collection using a standardized protocol. Barnes Jewish Hospital (Washington University School of Medicine) is a large high-volume center, which has enabled us to accrue specimens quickly. Nearly all sepsis patients in our bank have serial blood plasma and peripheral blood leukocytes collected daily in the ICU starting from day 1 of admission, with fully annotated paired clinical and survival data. We also have banked samples from ˜100 propensity-matched non-sepsis controls (IRB #201903142; PI: Aadel Chaudhuri). Overall, we have the necessary ground-truth data for studying cell-free DNA dynamics in sepsis patients with matched healthy donors.

We will perform WGBS on each of these serial plasma samples collected from sepsis patients, and perform bioinformatics analysis to determine: 1) The organs involved/damaged—by quantifying which organ tissue sources predominantly contribute to plasma cell-free DNA; 2) Dysfunctional status of the immune system—by quantifying exhausted T cell-derived cell-free DNA. To do these quantitations, we will deconvolve human-mapped reads from cell-free DNA WGBS using CIBERSORTx²⁷ with our custom signature matrix to determine relative abundances of each queried organ tissue type and dysfunctional/exhausted T cells, after normalizing out the predominant PBL-derived signal. We will correlate our predictions with ground-truth in our clinical cohort (Table 2, see Example 5). We will do this correlative analysis on a per-time-point basis, possible given the high level of clinical and laboratory annotation we have, and trend tissue- and exhausted T cell-specific cell-free DNA over time to correlate kinetics and dynamics with clinical ground-truth. To test the specificity of our approach, we will separately analyze blood plasma samples acquired from propensity score-matched controls. We will perform k-fold cross-validation to evaluate the generalizability of our results.

Through this analysis, we will model the kinetics and dynamics of organ tissue-specific cell-free DNA shed during sepsis, a major advance as the current literature only shows snapshots of this in isolated cases¹. Additionally, we will track the kinetics and dynamics of dysfunctional/exhausted T cells, expecting that the rise in dysfunctional/exhausted T cell-derived cell-free DNA precedes secondary infection in a significant subset of patients. We expect our findings to also shed light on spatiotemporal mechanisms of organ damage and immune exhaustion in sepsis, which should stimulate future research in efforts to ameliorate these primary drivers of sepsis-mediated morbidity and mortality. In addition to advancing our scientific understanding, we will generate sequencing data sets with paired clinical data that doesn't currently exist, which will serve as a valuable resource for the scientific community.

### Innovation

Here is outlined a two-pronged effort to shift our paradigm regarding sepsis dynamics through plasma cell-free DNA analysis. Specifically, 1) track the dynamics of organ-specific damage and 2) track the dynamics of T cell exhaustion during sepsis. The epigenomic cell-free DNA analysis approach we can use is novel for both the sepsis field and in a broader sense, as deconvolution of cell-free DNA whole genome bisulfite sequencing data for organ tissue and exhausted lymphocyte analysis has not been demonstrated before. The concept of quantifying exhausted lymphocytes from cell-free DNA data is a novel concept altogether. Still, the work we describe here, is supported by pre-existing literature supporting more elementary microarray-based approaches in individual snapshots/cases¹, as well as our own data.

We can also generate cell-free DNA sequencing data with paired clinical correlates from serially collected sepsis patients and propensity score-matched non-sepsis controls. These data don't currently exist, and will serve as a valuable resource for the scientific community, enabling our group and others to perform secondary analyses in order to further enhance our understanding of cell-free DNA derived temporal dynamics in sepsis, correlated with clinical parameters and outcomes. This data resource will be a major paradigm-shifting contribution to the sepsis and cell-free DNA genomics fields.

This technology can facilitate the development of noninvasive biomarkers to track sepsis patients. The results can further clarify how to develop and interpret these biomarkers, amplifying our understanding of when a septic patient is slipping into life-threatening multi-organ failure or developing increased risk for life-threatening secondary infection. We have already seen cell-free DNA biomarkers begin to be utilized in the sepsis field, such as the Karius assay, which enables rapid and noninvasive determination of infectious etiologies using a plasma whole genome sequencing approach²⁰. The sepsis field is absolutely ripe for improved precision diagnostic modalities, and the translational work described here can help facilitate that.

The technology described here can:
(1) use our new-found knowledge of the dynamics of immune exhaustion in sepsis patients in order to treat select patients with immunotherapy to boost their adaptive immune system at the precise times when it could reduce the risk of acquiring deadly secondary infections;
(2) use our new-found knowledge of the dynamics of organ tissue damage in sepsis in order to proactively introduce specific organ-protective measures in select sepsis patients to reduce the risk of deadly multi-organ failure;
(3) further our understanding of cell-free DNA epigenomics to determine residency location of exhausted immune cells—from which tissues vs. the periphery are they arising, which should enhance our scientific understanding by adding a spatial component to these cell-free DNA immunogenomics temporal/dynamics studies;
(4) perform machine learning to integrate data from our human-derived sequencing repository from this technology with clinical parameters to develop combinatorial biomarkers with even greater predictive potential. Ultimately this technology can be adapted to use cell-free DNA epigenomics to understand the multifactorial spatiotemporal underpinnings of sepsis, and by doing so to enable the development of precise biomarkers to improve patient outcomes.

Furthermore, the work described here can influence research in multiple different clinical fields. For example, in patients with inflammatory disorders, similar methodologies could be applied to noninvasively track tissue types and immune cell states, in order to anticipate potential flares and determine which organ tissues are being damaged by those flares. In patients undergoing deep wound healing, our research could potentially allow us to monitor this process precisely and noninvasively. Thus while our work in sepsis will be highly impactful, it has the potential to positively influence research in other clinical areas as well.

### REFERENCES

1. Moss, J., et al. Comprehensive human cell-type methylation atlas reveals origins of circulating cell-free DNA in health and disease. Nat Commun 9, 5068 (2018).
2. Rudd, K. E., et al. Global, regional, and national sepsis incidence and mortality, 1990-2017: analysis for the Global Burden of Disease Study. Lancet 395, 200-211 (2020).
3. Boomer, J. S., Green, J. M. & Hotchkiss, R. S. The changing immune system in sepsis: is individualized immuno-modulatory therapy the answer? Virulence 5, 45-56 (2014).
4. Cao, C., Yu, M. & Chai, Y. Pathological alteration and therapeutic implications of sepsis-induced immune cell apoptosis. Cell Death Dis 10, 782 (2019).
5. Delano, M. J. & Ward, P. A. The immune system's role in sepsis progression, resolution, and long-term outcome. Immunol Rev 274, 330-353 (2016).
6. Boomer, J. S., et al. Immunosuppression in patients who die of sepsis and multiple organ failure. JAMA 306, 2594-2605 (2011).
7. Denstaedt, S. J., Singer, B. H. & Standiford, T. J. Sepsis and Nosocomial Infection: Patient Characteristics, Mechanisms, and Modulation. Front Immunol 9, 2446 (2018).
8. Crowley, E., Di Nicolantonio, F., Loupakis, F. & Bardelli, A. Liquid biopsy: monitoring cancer-genetics in the blood. Nat Rev Clin Oncol 10, 472-484 (2013).
9. Chen, Y., et al. Clinical characteristics, risk factors, immune status and prognosis of secondary infection of sepsis: a retrospective observational study. BMC Anesthesiol 19, 185 (2019).
10. van Vught, L. A., et al. Incidence, Risk Factors, and Attributable Mortality of Secondary Infections in the Intensive Care Unit After Admission for Sepsis. JAMA 315, 1469-1479 (2016).
11. Boomer, J. S., Shuherk-Shaffer, J., Hotchkiss, R. S. & Green, J. M. A prospective analysis of lymphocyte phenotype and function over the course of acute sepsis. Crit Care 16, R112 (2012).
12. Chaudhuri, A. A., et al. Early Detection of Molecular Residual Disease in Localized Lung Cancer by Circulating Tumor DNA Profiling. Cancer Discov 7, 1394-1403 (2017).
13. Chin, R. I., et al. Detection of Solid Tumor Molecular Residual Disease (MRD) Using Circulating Tumor DNA (ctDNA). Mol Diagn Ther 23, 311-331 (2019).
14. Dang, H. X., et al. JCO Precision Oncology (2020).
15. Chaudhuri, A. A., et al. Emerging Roles of Urine-Based Tumor DNA Analysis in Bladder Cancer Management. JCO Precision Oncology 4, 808-817 (2020).
16. Swarup, V. & Rajeswari, M. R. Circulating (cell-free) nucleic acids—a promising, non-invasive tool for early detection of several human diseases. FEBS Lett 581, 795-799 (2007).
17. Sun, K., et al. Plasma DNA tissue mapping by genome-wide methylation sequencing for noninvasive prenatal, cancer, and transplantation assessments. Proc Natl Acad Sci USA 112, E5503-5512 (2015).
18. Rostami, A., et al. Senescence, Necrosis, and Apoptosis Govern Circulating Cell-free DNA Release Kinetics. Cell Rep 31, 107830 (2020).
19. Shen, S. Y., et al. Sensitive tumour detection and classification using plasma cell-free DNA methylomes. Nature 563, 579-583 (2018).
20. Blauwkamp, T. A., et al. Analytical and clinical validation of a microbial cell-free DNA sequencing test for infectious disease. Nat Microbiol 4, 663-674 (2019).
21. Bernstein, B. E., et al. The NIH Roadmap Epigenomics Mapping Consortium. Nat Biotechnol 28, 1045-1048 (2010).
22. Deng, J., et al. Targeted bisulfite sequencing reveals changes in DNA methylation associated with nuclear reprogramming. Nat Biotechnol 27, 353-360 (2009).
23. Gu, H., et al. Preparation of reduced representation bisulfite sequencing libraries for genome-scale DNA methylation profiling. Nat Protoc 6, 468-481 (2011).
24. Liu, M. C., Oxnard, G. R., Klein, E. A., Swanton, C. & Seiden, M. Response to W. C. Taylor, and C. Fiala and E. P. Diamandis. Ann Oncol (2020).
25. Zviran, A., et al. Genome-wide cell-free DNA mutational integration enables ultra-sensitive cancer monitoring. Nat Med 26, 1114-1124 (2020).
26. Newman, A. M., et al. Integrated digital error suppression for improved detection of circulating tumor DNA. Nat Biotechnol 34, 547-555 (2016).
27. Newman, A. M., et al. Determining cell type abundance and expression from bulk tissues with digital cytometry. Nat Biotechnol (2019).
28. Azad, T. D., et al. Circulating Tumor DNA Analysis for Detection of Minimal Residual Disease After Chemoradiotherapy for Localized Esophageal Cancer. Gastroenterology 158, 494-505 e496 (2020).
29. Chabon, J. J., et al. Integrating genomic features for non-invasive early lung cancer detection. Nature 580, 245-251 (2020).
30. Consortium, E. P. The ENCODE (ENCyclopedia Of DNA Elements) Project. Science 306, 636-640 (2004).
31. Martens, J. H. & Stunnenberg, H. G. BLUEPRINT: mapping human blood cell epigenomes. Haematologica 98, 1487-1489 (2013).
32. Juhling, F., et al. metilene: fast and sensitive calling of differentially methylated regions from bisulfite sequencing data. Genome Res 26, 256-262 (2016).
33. Wherry, E. J. & Kurachi, M. Molecular and cellular insights into T cell exhaustion. Nat Rev Immunol 15, 486-499 (2015).
34. Cohen, J. Statistical power analysis for the behavioral sciences, (L. Erlbaum Associates, Hillsdale, N. J., 1988).
35. Chaudhuri, A. A., et al. Oncomir miR-125b regulates hematopoiesis by targeting the gene Lin28A. Proc Natl Acad Sci USA 109, 4233-4238 (2012).
36. Chaudhuri, A. A., et al. MicroRNA-125b potentiates macrophage activation. J Immunol 187, 5062-5068 (2011).
37. O'Connell, R. M., Chaudhuri, A. A., Rao, D. S. & Baltimore, D. Inositol phosphatase SHIP1 is a primary target of miR-155. Proc Natl Acad Sci USA 106, 7113-7118 (2009).
38. O'Connell, R. M., et al. MicroRNAs enriched in hematopoietic stem cells differentially regulate long-term hematopoietic output. Proc Natl Acad Sci USA 107, 14235-14240 (2010).
39. Abbosh, C., Birkbak, N.J. & Swanton, C. Early stage NSCLC-challenges to implementing ctDNA-based screening and MRD detection. Nat Rev Clin Oncol 15, 577-586 (2018).
40. Barnell, E. K., et al. Noninvasive Detection of High-Risk Adenomas Using Stool-Derived Eukaryotic RNA Sequences as Biomarkers. Gastroenterology 157, 884-887 e883 (2019).

1. A method of determining cell type or cell states comprising:
(a) (i) providing or having been provided from a subject a sample comprising DNA and generating a methylation profile for the DNA in the sample; or (ii) providing or having been provided a methylation profile of the DNA in the sample, wherein the methylation profile comprises co-associated CpG methylation patterns and/or methylation haplotype blocks (MHBs) (tightly coupled CpG sites) of the DNA; and
(b) detecting cell type or cell state comprising:
(i) counting co-associated CpG methylation patterns in the DNA, wherein co-associated CpG methylation patterns comprises two or more CpGs in the DNA; or
(ii) counting MHBs;
(c) assigning the DNA to a cell type or cell state based on reference CpG values or reference MHB values, wherein reference CpG values or reference MHB values are determined from reference cell types or reference cell states; and
(d) counting DNA molecules assigned to each reference CpG value or reference MHB value, wherein each reference CpG value or reference MHB value corresponds to a cell type or a cell state.

2. The method of claim 1, further comprising
counting known single CpG methylation profiles to increase sensitivity;
determining cell state-specific signatures by the method of claim 1;
providing or having been provided cell state-specific signatures of the sample; or
administering a cancer treatment to the subject and measuring cell type and cell state in a sample as an indication of treatment response.

3. The method of claim 1, wherein the sample
is a blood plasma, tissue, or biopsy sample;
comprises a bodily fluid;
is selected from whole blood, plasma, urine, saliva, or stool;
does not comprise a solid tissue biopsy;
comprises cell-free DNA (cfDNA) and is blood from a subject having suspected of having, or at risk for having sepsis;
comprises a nucleic acid mixture comprising DNA, RNA, or any combination thereof; or
comprises cell-free DNA (cfDNA) and is collected from a tumor microenvironment.

4. The method of claim 1, wherein
reference values are differentially methylated CpGs derived from DNA originating from known cell types and known cell states or of bacterial, viral, fungal, or eukaryotic parasitic origin;
the methylation profile for the DNA in the sample is generated using microarrays or bisulfite sequencing;
the subject has been administered immunotherapy prior to providing a sample;
if ctilDNA levels are decreased compared to ctilDNA levels in a responder to immunotherapy, the subject is determined to be at risk for being a non-responder to immunotherapy;
the DNA is cell-free DNA and is plasma-derived;
the DNA is cell-free tumor ctDNA;
the DNA is cell-free and a rare cell type circulating DNA; or
the DNA is classified as originating from a normal leukocyte cell, a tumor-associated cell, or a tumor infiltrating leukocyte.

5-12. (canceled)

13. The method of claim 3, wherein the tumor microenvironment comprises tumor infiltrating leukocytes.

14-15. (canceled)

16. The method of claim 1, wherein the cell state measured is from DNA from a circulating, cell-free tumor infiltrating leukocyte (TIL) or the sample is a sample from a tumor microenvironment (TME).

17. The method of claim 16, comprising:
profiling TILs according to methylation signatures; and
determining proportions of distinct TIL subsets from a cell type-specific methylation profile identified in the cell-free DNA.

18-23. (canceled)

24. The method of claim 1, wherein the sample is a blood sample from a subject having, suspected of having, or at risk for having sepsis.

25. The method of claim 24, wherein
exhausted lymphocyte cell states, exhausted T cells, organ-specific cell states, or organ-specific cell types are measured; or
the DNA originates or an organ, a damaged organ, a T cell, exhausted T cells, an immune cell, a microbe, septic tissue, or a secondary infection site.

26-28. (canceled)

29. The method of claim 24, wherein if cfDNA analysis detects:
(i) DNA originating from a microbial pathogen, the subject is diagnosed with an infection or sepsis;
(ii) reduced cfDNA originating from a microbial pathogen compared to the cfDNA originating from a microbial pathogen, and the subject is administered a treatment optionally an antibiotic, the subject is determined to be responding to treatment;
(ii) reduced cfDNA from a microbial pathogen compared to the cfDNA analysis measured at an earlier time it is determined that the subject is responding to a treatment or an infection is improving;
(iv) elevated cfDNA from an organ tissue, an infection source is determined to be the organ tissue with elevated detected cfDNA;
(v) elevated cfDNA from an organ tissue or multiple organ systems suspected of being damaged compared to a control, the organ is determined to be damaged or the subject is determined to be at risk for multi-organ failure;
(vi) reduced cfDNA from a damaged organ tissue compared to the cfDNA analysis measured at an earlier time, it is determined that the organ damage is improving; or
(vii) elevated cfDNA from exhausted T cells or an opportunistic pathogen compared to a control, the subject is determined to be at risk for a secondary infection.

30-37. (canceled)

38. A computer-aided method for detecting at least one abundance of at least one cell identity in a biological sample, the sample comprising DNA, the method comprising:
providing a plurality of reads, each read comprising a sequence of the DNA and associated methylation status;
providing a CpG library comprising a plurality of entries, each entry comprising a CpG site and a corresponding cell identity, each CpG site comprising a co-associated CpG site, and each corresponding cell identity comprising a cell type or a cell state;
transforming, using a computing device, the plurality of reads into a plurality of read assignments according to at least one assignment rule, each read assignment comprising one of a cell identity, a cell-related identity, and an unrelated identity; and
transforming, using the computing device, the plurality of read assignments into the at least one abundance, each abundance corresponding to one cell identity, each abundance comprising a total number of read assignments comprising the one cell identity.

39. The computer-aided method of claim 38, wherein the at least one assignment rule comprises at least one of:
transforming, using the computing device, the read into the cell-related identity if the read comprises no more than one CpG site from the plurality of entries of the CpG library;
transforming, using the computing device, the read into the cell identity if the read comprises at least two CpG sites from the plurality of entries of the CpG library with the same corresponding cell identity; and
transforming, using the computing device, the read into the unrelated identity if the read does not comprise any CpG site from the plurality of entries of the CpG library.

40. The computer-aided method of claim 38, further comprising transforming, using the computing device, each abundance into at least one of a relative abundance and an absolute abundance, wherein:
each relative abundance comprises the abundance of one cell identity normalized by the total of all abundances of all cell identities; and
each absolute abundance comprises the abundance of one cell identity normalized by a sum of the abundance and the total number of read assignments.

41. The computer-aided method of claim 38, wherein
the DNA comprises cell-free DNA;
providing the plurality of reads further comprises performing bisulfite sequencing or microarray methylation profiling on the DNA;
each CpG site is differentially methylated within cells of one cell identity and each co associated CpG site comprises a sequence position proximal to at least one additional CpG site with the same corresponding cell identity; or
the biological sample comprises a bodily fluid;
the biological sample is selected from whole blood, plasma, urine, saliva, and stool; or
the biological sample does not comprise a solid tissue biopsy.

42-43. (canceled)

44. The computer-aided method of claim 38, wherein providing the CpG library further comprises:
providing a plurality of isolated DNA corresponding to one cell identity;
performing bisulfite sequencing or microarray methylation profiling on the plurality of isolated DNA to obtain a plurality of isolated reads, each isolated read comprising an isolated sequence of an isolated DNA and associated methylation status;
performing differential methylated region analysis on the plurality of isolated reads to identify a plurality of candidate CpG sites; and
assigning a candidate CpG site as an entry of the CpG library for the one cell identity if the candidate CpG site comprises a sequence position proximal to at least one additional candidate CpG site.

45-55. (canceled)

56. A computer-aided method for detecting at least one abundance of at least one cell identity in a biological sample, the sample comprising DNA, the method comprising:
providing a plurality of reads, each read comprising a sequence of the DNA and associated methylation status;
providing a Methylation Haplotype Block (MHB) library comprising a plurality of entries, each entry comprising an MHB and a corresponding cell identity, each MHB comprising at least two co-associated CpG sites, and each corresponding cell identity comprising a cell type or a cell state;
transforming, using a computing device, the plurality of reads into a plurality of read assignments according to at least one assignment rule, each read assignment comprising one of a cell identity, a cell-related identity, and an unrelated identity; and
transforming, using the computing device, the plurality of read assignments into the at least one abundance, each abundance corresponding to one cell identity, each abundance comprising a total number of read assignments comprising the one cell identity.

57. The computer-aided method of claim 56, wherein the at least one assignment rule comprises transforming, using the computing device, the read into the cell identity if the read comprises at least one MHB from the plurality of entries of the MHB library with the corresponding cell identity.

58. The computer-aided method of claim 56, further comprising transforming, using the computing device, each abundance into a relative abundance, wherein each relative abundance comprises the abundance of one cell identity normalized by the total of all abundances of all cell identities.

59. The computer-aided method of claim 56, wherein
the DNA comprises cell-free DNA;
each MHB site comprises at least two differentially methylated CpG sites proximal to each other within cells of one cell identity;
the biological sample comprises a bodily fluid;
the biological sample is selected from whole blood, plasma, urine, saliva, and stool;
the biological sample does not comprise a solid tissue biopsy; or
providing the plurality of reads further comprises performing bisulfite sequencing or microarray methylation profiling on the DNA.

60-61. (canceled)

62. The computer-aided method of claim 56, wherein providing the MHB library further comprises:
providing a plurality of isolated DNA corresponding to one cell identity;
performing bisulfite sequencing or microarray methylation profiling on the plurality of isolated DNA to obtain a plurality of isolated reads, each isolated read comprising an isolated sequence of isolated DNA and associated methylation status;
performing differential methylated region analysis on the plurality of isolated reads to identify a plurality of candidate CpG sites; and
assigning each sequence including at least two candidate CpG sites proximal to each other as an MHB corresponding to the one cell identity in the MHB library for the one cell identity.

63-77. (canceled)
